(19) United States
(12) Patent Application Publication
(10) Pub. No.: US 2025/0230449 A1
(43) Pub. Date: Jul. 17, 2025

(54) GENELIGHT CULTURES AND EXTRACTS AND APPLICATIONS THEREOF

(71) Applicant: Bio Capital Holdings, LLC, Houston, TX (US)

(72) Inventor: Raul CUERO RENGIFO, Cypress, TX (US)

(21) Appl. No.: 19/013,103

(22) Filed: Jan. 8, 2025

Related U.S. Application Data
(60) Provisional application No. 63/619,823, filed on Jan. 11, 2024.

(51) Int. Cl. C12N 15/81; C12N 1/18; C12N 1/20; C12N 9/02; C12N 15/52; C12N 15/74

(52) U.S. Cl. CPC C12N 15/81; C12N 1/18; C12N 1/20; C12N 9/0067; C12N 15/52; C12N 15/74; C12Y 112/99006; C12N 2510/02

(57) ABSTRACT

Described herein are genelight cultures and extracts and methods of making and using thereof. In one aspect, the method of making a genelight culture or extract includes the steps of (a) making a DNA construct containing genes for producing a heat shock protein, RuBisCO large subunit 1, tonB, phosphoenol pyruvate carboxykinase, phosphoenol pyruvate carboxylase, hydrogenase, p-type ATPase, and, in some aspects, enhanced green fluorescent protein, (b) introducing the DNA construct into host microbial cells via transformation or transfection, and (c) culturing the microbial cells to produce the genelight cultures and extracts. The compositions of these cultures and extracts can be tailored to have specific properties such as the ability to provide power to a light emitting diode. The cultures and extracts have further uses including enhancing the growth of plants and as supplemental nutrients of cultures of industrially important microorganisms. The cultures and extracts further have UV-protective properties.

### CROSS-REFERENCE TO RELATED APPLICATIONS

This application claims the benefit of U.S. Provisional Application Ser. No. 63/619,823, filed Jan. 11, 2024, which is incorporated herein by reference in its entirety.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The sequence listing in written computer readable format (CRF) as a text file named “930201-1170_Sequence_Listing.xml” created on Dec. 26, 2024, and having a size of 407,521 bytes, is incorporated by reference in its entirety.

### BACKGROUND

Battery-powered devices are useful in applications ranging from telecommunications to medical devices to providing light during power outages. Current battery technology has several drawbacks, however. Although alkaline batteries can be recycled, few facilities exist for doing so and many end up in landfills each year. Alkaline batteries are also prone to leakage as they age and are used, which can ruin electronic devices. Lead-acid batteries are bulky, contain toxic metals, and may overheat during charging; they may also leak electrolytes (including corrosive acids) under improper storage conditions. Lithium-ion batteries are typically small and rechargeable but charge cycles are limited and transportation restrictions exist due to the possibility of short circuits leading to fires.

It would be advantageous to develop a new, inexpensive, portable power source that is effective and poses no environmental hazards during the disposal process. It would further be advantageous if this power source could make use of water-based electrolyte solutions and did not present a fire hazard during transportation or storage. It would further be advantageous if the raw materials used to generate the electrolyte solutions, or the electrolyte solutions themselves had other applications in agriculture, including plant tissue culture applications; commercial microorganism culture, including growth of organisms that produce industrially important compounds such as ethanol, acetic acid, rennet, insulin, and related compounds; and the like, either as a separate use or as a use for electrolyte solutions that have aged out of useful life. The present invention addresses these needs.

### SUMMARY

Described herein are genelight cultures and extracts and methods of making and using thereof. In one aspect, the method of making a genelight culture or extract includes the steps of (a) making a DNA construct containing genes for producing a heat shock protein, RuBisCO large subunit 1, tonB, phosphoenol pyruvate carboxykinase, phosphoenol pyruvate carboxylase, hydrogenase, p-type ATPase, and, in some aspects, enhanced green fluorescent protein, (b) introducing the DNA construct into host microbial cells via transformation or transfection, and (c) culturing the microbial cells to produce the genelight cultures and extracts. The compositions of these cultures and extracts can be tailored to have specific properties such as the ability to provide power to a light emitting diode and to other small devices. The cultures and extracts have further uses including enhancing the growth of plants, increasing the production of plant hormones, increasing the production of phytochrome proteins and chlorophyll, and as supplemental nutrients of cultures of industrially important microorganisms. The cultures and extracts further have UV-protective properties.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

Many aspects of the present disclosure can be better understood with reference to the following drawings, which are incorporated in and constitute a part of this specification. The components in the drawings are not necessarily to scale, emphasis instead being placed upon clearly illustrating the principles of the present disclosure.

FIGS. 1A-1B show, respectively, linear and circular maps of an exemplary biological device.

FIGS. 2A-2B show, respectively, linear and circular maps of an exemplary biological device.

FIGS. 3A-3B show, respectively, linear and circular maps of an exemplary biological device.

FIG. 4 shows the expression of phytochrome in grasses that have been treated with an extract/lysate from a device having SEQ ID NO. 10, a device having SEQ ID NO. 30, non-transformed yeast, or a water control. The extract/lysate from devices having SEQ ID NO. 10 causes field grass to produce more phytochrome over a two-week treatment period compared to other treatments and/or controls.

FIG. 5A shows grass prior to treatment with a disclosed device, while FIG. 5B shows the same grass after 20 days of treatment with a disclosed device.

FIG. 6 shows CO₂ in ppm measured for grass treated with disclosed and control compositions.

FIG. 7 shows a time series of CO₂ measured for grass after different treatment time periods.

FIGS. 8A-8B show tomato plants after 1 month in the field (FIG. 8A) and after a second tomato harvest in the field (FIG. 8B). Plants, from left to right, include water control, plants treated with non-transformed yeast, plants treated with a polyactive carbohydrate extract, plants treated with a lysate/extract from a disclosed biological device including SEQ ID NO. 30, and plants treated with a lysate/extract from a disclosed biological device including SEQ ID NO. 10.

FIG. 9A shows the mean weight per tomato produced by the plants shown in FIGS. 8A-8B. FIG. 9B shows the total weight of tomatoes produced in each harvest for the plants shown in FIGS. 8A-8B. FIG. 9C shows the total number of tomatoes produced in each harvest for the plants shown in FIGS. 8A-8B.

FIGS. 10A-10E show photos of the plants subjected to the treatments described in FIGS. 8A-8B as well as exemplary tomato harvests from the same.

FIG. 11 shows protein concentration from germinated corn seeds treated with lysate/extract from a disclosed biological device including SEQ ID NO. 30 and corn seeds treated with a lysate/extract from a disclosed biological device including SEQ ID NO. 10, as well as a non-transformed yeast control.

FIGS. 12A-12B show, respectively, early germination percentage and root length for corn seeds treated with lysate/extract from a disclosed biological device including SEQ ID NO. 30 and corn seeds treated with a lysate/extract from a disclosed biological device including SEQ ID NO. 10, as well as a water control and a non-transformed yeast control.

FIGS. 13A-13C show, respectively, use of a circuit including a disclosed lysate/extract (from E. coli transformed with SEQ ID NO. 9) powering a white LED, a red LED, and a yellow LED.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are DNA constructs containing the following genetic components:
(a) a gene that encodes a heat shock protein;
(b) a gene that encodes RuBisCO large subunit 1;
(c) a gene that encodes tonB;
(d) a gene that encodes phosphoenol pyruvate carboxykinase;
(e) a gene that encodes phosphoenol pyruvate carboxylase;
(f) a gene that encodes a hydrogenase; and
(g) a gene that encodes a p-type ATPase.

The DNA constructs may variously encode genes encoding reporter proteins, genes encoding resistance to one or more antibiotics, and the like, and may include regulatory sequences including promoters, terminators, ribosomal binding sites, LAC operons, or other components necessary for the replication of and expression of the genes encoded by the DNA constructs inside microbial hosts such as, for example, Saccharomyces cerevisiae, Escherichia coli, and other microorganisms. Also disclosed are vectors including the DNA constructs and biological devices consisting of host cells that include one or more copies of the vectors.

Described herein are genelight cultures and extracts and methods of making and using thereof. In one aspect, the method of making a genelight culture or extract includes the steps of (a) making a DNA construct containing genes for producing a heat shock protein, RuBisCO large subunit 1, tonB, phosphoenol pyruvate carboxykinase, phosphoenol pyruvate carboxylase, hydrogenase, and a P-type ATPase, (b) introducing the DNA construct into host microbial cells via transformation or transfection, and (c) culturing the microbial cells to produce the genelight cultures and extracts. The cultures are grown in standard media for host cells such as, for example, E. coli or S. cerevisiae. The compositions of these cultures and extracts can be tailored to have specific properties such as, for example, the ability to provide power to a light emitting diode (LED) with a specific voltage, or to power other small electronic devices. In some aspects, extracts from or cultures of Acidithiobacillus ferrooxidans can be used in combinations with the disclosed devices and cultures in order to power lights, LEDs, and other small electronic devices. The cultures and extracts have further uses including enhancing the growth of plants, including plants grown from tissue culture, and as supplemental nutrients of cultures of industrially, commercially, and/or scientifically important microorganisms. The cultures and extracts further have UV-protective and/or UV-blocking properties and can be incorporated into or applied on various materials, surfaces, and human or animal subjects for the purpose of protecting those materials, surfaces, and human or animal subjects from the harmful effects of radiation.

As used herein, a “genelight extract” is an extract produced by the biological devices disclosed herein. In one aspect, the genelight extract contains the biological devices in either a lysed or a whole cell state. In another aspect, the genelight extract contains proteins encoded by the genes in the DNA constructs disclosed herein, or contains the catalytic products of these proteins, or a combination thereof. In one aspect, the genelight extract is capable of providing power to a light source and/or to another small physical device. In a further aspect, the genelight extract can be used to enhance the growth of plants, including plants grown from tissue culture, or can be used to provide supplemental nutrients for cultures of industrially, commercially, and/or scientifically important microorganisms. In some aspects, the genelight extracts can enhance production of plant hormones including auxin, abscisic acid (ABA), jasmonic acid, and salicylic acid, as well as enhance or increase production of other important products including phytochrome proteins, chlorophyll, lycopene, carotene and other forms of vitamin A, and vitamin C. In another aspect, the genelight extracts can be used for detection of plant hormones including auxin, abscisic acid (ABA), jasmonic acid, and salicylic acid. In any of these aspects, the genelight extracts can be applied to plants and/or used for other purposes as liquids, as powders, or can be incorporated into plant tissue culture or the soil in which plants grow as pellets.

Before the present compounds, compositions, articles, devices, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In this specification and in the claims that follow, reference will be made to a number of terms that shall defined to have the following meanings:

It must be noted that, as used in the specification and the appended claims, the singular forms “a,” “an,” and “the” include plural referents unless the context clearly dictates otherwise. Thus, for example, reference to “a metabolite” includes mixtures of two or more such metabolites, and the like.

“Optional” or “optionally” means that the subsequently described event or circumstance can or cannot occur, and that the description includes instances where the event or circumstance occurs and instances where it does not. For example, the phrase “a microorganism is optionally genetically modified” means that the microorganism may or may not be genetically modified.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

As used herein, the term “about” is used to provide flexibility to a numerical range endpoint by providing that a given numerical value may be “a little above” or “a little below” the endpoint without affecting the desired result. For purposes of the parent disclosure, “about” refers to a range extending from 10% below the numerical value to 10% above the numerical value. For example, if the numerical value is 10, “about 10” means between 9 and 11, inclusive of the endpoints 9 and 11.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less' and should be interpreted to include the specific ranges of ‘about x,’ ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed that while specific reference to each various individual and collective combination and permutation of these compounds may not be explicitly disclosed, each is specifically contemplated and described herein. For example, if a UV protective genelight extract is disclosed and discussed and a number of different additional UV protective compounds are discussed, each and every combination and permutation of UV protective genelight extract and UV protective compound that is possible is specifically contemplated unless specifically indicated to the contrary. For example, if a class of molecules A, B, and C are disclosed as well as a class of molecules D, E, and F, and an example of a combination molecule, A-D, is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the subgroup of A-E, B-F, and C-E is specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed, it is understood that each of these additional steps can be performed with any specific embodiment or combination of elements of the disclosed methods, and that each such combination is specifically contemplated and should be considered disclosed.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

### DNA Constructs and Biological Devices

In one aspect, cells transformed with a DNA construct can be used in the methods described herein. It is understood that one way to define the variants and derivatives of the genetic components and DNA constructs described herein is in terms of homology/identity to specific known sequences. Those of skill in the art readily understand how to determine the homology of two nucleic acids. For example, the homology can be calculated after aligning two sequences so that the homology is at its highest level. Another way of calculating homology can be performed according to published algorithms (see Zuker, M., Science, 244:48-52, 1989; Jaeger et al, Proc. Natl. Acad. Sci. USA, 86:7706-7710, 1989; Jaeger et al, Methods Enzymol., 183:281-306, 1989, which are herein incorporated by reference for at least material related to nucleic acid alignment).

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of mutations and homology can be combined together in any combination, such as embodiments that have at least about 70%, about 75%, about 80%, about 85%, about 90%, about 95%, or about 99% homology to a particular sequence wherein the variants are conservative or silent mutations. It is understood that any of the sequences described herein can be a variant or derivative having the homology values listed above.

In some aspects, genes of interest can be spliced into suitable vectors using restriction enzymes and/or other techniques known in the art. Further in this aspect, synthesis and/or isolation of the genes of interest prior to inclusion in the disclosed constructs may result in the addition of C-terminal and/or N-terminal sequence data including, but not limited to, restriction enzyme recognition sites, linking bases, short segments of chromosomal DNA (including introns or portions of introns if the sequences originate from eukaryotic cells), transposons, nucleotide repeats, regulatory sequences, and/or other material that do not contribute to the known structure of the expressed protein, or are not part of the expressed protein's active site. In one aspect, presence of these remnants may lead to somewhat reduced homology with respect to gene sequence, but the DNA constructs encoding the same can still produce proteins having the desired sequence, active site, and function.

In another aspect, many eukaryotic genes include introns and mRNAs produced during transcription of the same can be spliced differently, producing several transcript variants from the same gene but having slightly different sequences (i.e., reduced levels of homology). In one aspect, different transcript variants can produce proteins having the same active site but differing in another way (e.g. in C-terminal or N-terminal sequence, affecting assembly of protein subunits or other folding processes, cellular localization of the peptides or proteins, or activity level of the peptides or proteins produced due to differential regulation, or the like.

In one aspect, a database such as, for example, GenBank, can be used to determine the sequences of genes and/or regulatory regions of interest, the species from which these elements originate, and related homologous sequences.

In one aspect, the nucleic acids used in the DNA constructs described herein can be amplified using polymerase chain reaction (PCR) prior to being ligated into a plasmid or other vector. Typically, PCR-amplification techniques make use of primers, or short, chemically-synthesized oligonucleotides that are complementary to regions on each respective strand flanking the DNA or nucleotide sequence to be amplified. A person having ordinary skill in the art will be able to design or choose primers based on the desired experimental conditions. In general, primers should be designed to provide for both efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the skilled practitioner with primer design. Primers can optionally incorporate restriction enzyme recognition sites at their 5′ ends to assist in later ligation into plasmids or other vectors.

PCR can be carried out using purified DNA, unpurified DNA that is integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase, such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or genes. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In a further aspect, the plasmid can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of that coding sequence.

As used herein, “expression” refers to transcription and/or accumulation of an mRNA derived from a gene or DNA fragment. Expression may also be used to refer to translation of mRNA into a peptide, polypeptide, or protein.

### Genelight DNA Constructs

In one aspect, provided herein are DNA constructs having at least the following genetic components:
(a) a gene that encodes a heat shock protein;
(b) a gene that encodes RuBisCO large subunit 1;
(c) a gene that encodes tonB;
(d) a gene that encodes phosphoenol pyruvate carboxykinase;
(e) a gene that encodes phosphoenol pyruvate carboxylase;
(f) a gene that encodes a hydrogenase; and
(g) a gene that encodes a p-type ATPase.

Each component of the DNA constructs is described in detail below. The components can be present in any order.

In one aspect, the DNA constructs disclosed herein incorporate a gene that encodes a heat shock protein. In a further aspect, heat shock proteins are a group of proteins produced by cells in response to exposure to stressful conditions. In a further aspect, heat shock proteins can be expressed in response to heat shock but also to cold, UV light, wound healing, exposure to toxic chemicals such as, for example heavy metals, as well as during tissue remodeling. In a further aspect, a heat shock protein may function as a chaperone protein by assisting in the refolding process of proteins damaged by cell stress. In a further aspect, the heat shock protein can be HSP60, HSP70, or HSP90, where the number refers to the size in kilodaltons of the protein. In one aspect, the heat shock protein is HSP70 and has a weight of about 70 kDa.

In one aspect, the gene that encodes a heat shock protein is isolated from a yeast such as, for example, Saccharomyces cerevisiae, including, but not limited to, strain EC1118, S288C, YJM1242, YJM1326, YJM1338, YJM1355, YJM1356, YJM1387, YJM1401, YJM1415, YJM1447, YJM1477, YJM1478, YJM1526, YJM1527, YJM320, YJM450, YJM453, YJM554, YJM627, YJM683, YJM969, YJM978, YJM981, YJM987, YJM993 In a further aspect, the gene that encodes a heat shock protein has SEQ ID NO. 1 or at least 70% homology thereto, at least 75% homology thereto, at least 80% homology thereto, at least 85% homology thereto, at least 90% homology thereto, at least 95% homology thereto, or at least 99% homology thereto.

Other sequences encoding HSP70 or related or homologous genes can be identified in a database such as, for example, GenBank. In one aspect, the gene that encodes HSP7 is isolated from Saccharomyces cerevisiae and can be identified by the GI number P046084.1 in the GenBank database. In another aspect, sequences useful herein include those with GI numbers listed in Table 1:

TABLE 1
Heat Shock Protein 70

| | Sequence | |
|---|---|---|
| Source Organism | Description | GI Number |
| Saccharomyces cerevisiae | Genomic DNA | CP046084.1 |
| Saccharomyces cerevisiae | Genomic DNA | CP036483.1 |
| Saccharomyces cerevisiae | Genomic DNA | CP033473.1 |
| Saccharomyces cerevisiae | Genomic DNA | CP029160.1 |
| Saccharomyces cerevisiae | Genomic DNA | CP026298.1 |
| Saccharomyces cerevisiae | Genomic DNA | CP022969.1 |
| Saccharomyces cerevisiae | Genomic DNA | CP020126.1 |
| Saccharomyces cerevisiae | Genomic DNA | CP004710.2 |
| YJM1326 | | |
| Saccharomyces cerevisiae | Genomic DNA | CP127198.1 |
| Saccharomyces cerevisiae | Genomic DNA | CP011550.1 |
| Saccharomyces cerevisiae | Genomic DNA | AP026835.1 |
| Saccharomyces cerevisiae | Genomic DNA | CP011671.1 |
| Saccharomyces cerevisiae | Genomic DNA | CP089103.1 |
| Saccharomyces paradoxus | Genomic DNA | CP081969.2 |
| Saccharomyces cerevisiae | Genomic DNA | CP063258.1 |
| Saccharomyces cerevisiae | Genomic DNA | CP059525.2 |
| Saccharomyces cerevisiae | Genomic DNA | BK006938.2 |
| S288C | | |
| Saccharomyces cerevisiae | Genomic DNA | NM_001180289.1 |
| S288C | | |
| Saccharomyces cerevisiae | Genomic DNA | FN393064.1 |
| EC1118 | | |
| Saccharomyces cerevisiae | Genomic DNA | Z74277.1 |
| Saccharomyces cerevisiae | Heat shock | X13713.1 |
| | cognate gene | |
| synthetic construct | Genomic DNA | EF058944.1 |
| Saccharomyces cerevisiae | Genomic DNA | CP025100.1 |
| Saccharomyces cerevisiae | Genomic DNA | CP020228.1 |
| Saccharomyces cerevisiae | Genomic DNA | CP020160.1 |
| Saccharomyces cerevisiae | Genomic DNA | CP004738.2 |
| YJM1447 | | |
| Saccharomyces cerevisiae | Genomic DNA | CP004688.2 |
| YJM981 | | |
| Saccharomyces cerevisiae | Genomic DNA | CP004678.2 |
| YJM627 | | |
| Saccharomyces cerevisiae | Genomic DNA | CP004727.2 |
| YJM1401 | | |
| Saccharomyces cerevisiae | Genomic DNA | CP004717.2 |
| YJM1356 | | |
| Saccharomyces cerevisiae | Genomic DNA | CP004687.2 |
| YJM978 | | |
| Saccharomyces cerevisiae | Genomic DNA | CP004667.2 |
| YJM320 | | |
| Saccharomyces cerevisiae | Genomic DNA | CP004746.2 |
| YJM1527 | | |
| Saccharomyces cerevisiae | Genomic DNA | CP004716.2 |
| YJM1355 | | |
| Saccharomyces cerevisiae | Genomic DNA | CP004676.2 |
| YJM554 | | |
| Saccharomyces cerevisiae | Genomic DNA | CP008239.1 |
| Saccharomyces cerevisiae | Genomic DNA | CP008324.1 |
| Saccharomyces cerevisiae | Genomic DNA | CP008273.1 |
| Saccharomyces cerevisiae | Genomic DNA | CP008256.1 |
| Saccharomyces cerevisiae | Genomic DNA | CP008222.1 |
| Saccharomyces cerevisiae | Genomic DNA | CP008409.1 |
| Saccharomyces cerevisiae | Genomic DNA | CP008392.1 |
| Saccharomyces cerevisiae | Genomic DNA | CP008375.1 |
| Saccharomyces cerevisiae | Genomic DNA | CP008358.1 |
| Saccharomyces cerevisiae | Genomic DNA | CP008341.1 |
| Saccharomyces cerevisiae | Genomic DNA | CP008494.1 |
| Saccharomyces cerevisiae | Genomic DNA | CP008443.1 |
| Saccharomyces cerevisiae | Genomic DNA | CP008579.1 |
| Saccharomyces cerevisiae | Genomic DNA | CP008511.1 |
| Saccharomyces cerevisiae | Genomic DNA | CP008647.1 |
| Saccharomyces cerevisiae | Genomic DNA | CP008630.1 |
| Saccharomyces cerevisiae | Genomic DNA | CP008596.1 |
| Saccharomyces cerevisiae | Genomic DNA | CP008188.1 |
| Saccharomyces cerevisiae | Genomic DNA | CP008171.1 |
| Saccharomyces cerevisiae | Genomic DNA | CP008154.1 |
| Saccharomyces cerevisiae | Genomic DNA | CP008681.1 |
| Saccharomyces cerevisiae | Genomic DNA | CP008137.1 |
| Saccharomyces cerevisiae | Genomic DNA | CP008120.1 |
| Saccharomyces cerevisiae | Genomic DNA | CP008086.1 |
| Saccharomyces cerevisiae | Genomic DNA | CP008052.1 |
| Saccharomyces cerevisiae | Genomic DNA | CP008035.1 |
| Saccharomyces cerevisiae | Genomic DNA | CP008001.1 |
| Saccharomyces cerevisiae | Genomic DNA | CP007984.1 |
| Saccharomyces cerevisiae | Genomic DNA | CP007950.1 |
| Saccharomyces cerevisiae | Genomic DNA | CP007899.1 |
| Saccharomyces cerevisiae | Genomic DNA | CP007882.1 |
| Saccharomyces cerevisiae | Genomic DNA | CP007831.1 |
| Saccharomyces cerevisiae | Genomic DNA | CP133024.1 |
| Saccharomyces cerevisiae | Genomic DNA | CP004745.2 |
| YJM1526 | | |
| Saccharomyces cerevisiae | Genomic DNA | CP004684.2 |
| YJM969 | | |
| Saccharomyces cerevisiae | Genomic DNA | CP004743.2 |
| YJM1478 | | |
| Saccharomyces cerevisiae | Genomic DNA | CP004713.2 |
| YJM1338 | | |
| Saccharomyces cerevisiae | Genomic DNA | CP004692.2 |
| YJM993 | | |
| Saccharomyces cerevisiae | Genomic DNA | CP004742.2 |
| YJM1477 | | |
| Saccharomyces cerevisiae | Genomic DNA | CP004722.2 |
| YJM1387 | | |
| Saccharomyces cerevisiae | Genomic DNA | CP004672.2 |
| YJM453 | | |
| Saccharomyces cerevisiae | Genomic DNA | CP004701.2 |
| YJM1242 | | |
| Saccharomyces cerevisiae | Genomic DNA | CP004681.2 |
| YJM683 | | |
| Saccharomyces cerevisiae | Genomic DNA | CP004690.2 |
| YJM987 | | |
| Saccharomyces cerevisiae | Genomic DNA | CP004670.2 |
| YJM450 | | |
| Saccharomyces cerevisiae | Genomic DNA | CP011082.1 |
| Saccharomyces cerevisiae | Genomic DNA | CP004729.1 |
| YJM1415 | | |
| Saccharomyces cerevisiae | Genomic DNA | CP072078.1 |
| Saccharomyces cerevisiae | Genomic DNA | CP072094.1 |
| Saccharomyces cerevisiae | Genomic DNA | CP093816.1 |
| Saccharomyces cerevisiae | Genomic DNA | CP093752.1 |
| Saccharomyces cerevisiae | Genomic DNA | CP093704.1 |
| Saccharomyces cerevisiae | Genomic DNA | CP093688.1 |
| Saccharomyces cerevisiae | Genomic DNA | CP093672.1 |
| Saccharomyces cerevisiae | Genomic DNA | CP093656.1 |
| Saccharomyces cerevisiae | Genomic DNA | CP093624.1 |
| Saccharomyces cerevisiae | Genomic DNA | CP093576.1 |
| Saccharomyces cerevisiae | Genomic DNA | CP093560.1 |
| Saccharomyces cerevisiae | Genomic DNA | LR813588.2 |
| Saccharomyces cerevisiae | Genomic DNA | LR813537.2 |
| Saccharomyces cerevisiae | Heat shock | M25395.1 |
| | protein 70 | |
| Saccharomyces cerevisiae | Genomic DNA | CP046471.1 |
| Saccharomyces cerevisiae | Genomic DNA | CP125417.1 |
| Saccharomyces cerevisiae | Genomic DNA | CP125400.1 |
| Saccharomyces cerevisiae | Genomic DNA | CP080606.1 |

In one aspect, the DNA constructs disclosed herein include a gene that expresses ribulose-1,5-bisphosphate carboxylase/oxygenase (RuBisCO) large subunit 1. RuBisCO is an enzyme involved in carbon fixation and can be isolated from plants, algae, cyanobacteria, and phototrophic and/or chemoautotrophic proteobacteria. In a further aspect, the RuBisCO large subunit is typically encoded by chloroplast DNA. In a still further aspect, RuBisCO catalyzes the formation of two molecules of glycerate-3-phosphate from ribulose-1,5-bisphosphate and carbon dioxide. In an alternative aspect, RuBisCO is capable of catalyzing the formation of phosphoglycolate and 3-phosphoglycerate from ribulose-1,5-bisphosphate and molecular oxygen.

In one aspect, the gene that encodes RuBisCO large subunit 1 is isolated from an algae, protist, or stramenophile such as, for example, a Botrydiopsis sp., a Botrydium sp. (e.g. Botrydium granulatum or Botrydium stoloniferum), Bumilleriopsis sp. SAG 33.93, a Chroomonas sp. (e.g. Chroomonas collegionis, Chroomonas debatzensis, Chroomonas mesostigmatica CCMP1168, or Chroomonas placoidea), Chrysoparadoxa australica, a Cryptomonas sp. (e.g. Cryptomonas borealis, Cryptomonas curvata, Cryptomonas erosa, Cryptomonas gyropyrenoidosa, Cryptomonas marssonii, Cryptomonas obovoidea, Cryptomonas ovata, Cryptomonas pyrenoidifera, Cryptomonas sp. CCAC 0109, Cryptomonas sp. NIES-1327, Cryptomonas sp. NIES-345, Cryptomonas sp. NIES-3952, or Cryptomonas tetrapyrenoidosa), a Cryptophyta sp. (e.g. Cryptophyta sp. CCMP2293 or Cryptophyta sp. ECY-2019a), Erythrolobus coxiae, Erythrotrichia foliiformis, Guillardia theta, Hanusia phi, a Hemiselmis sp. (e.g. Hemiselmis andersenii, Hemiselmis cryptochromatica, or Hemiselmis tepida), Heterothrix mucicola, an Ophiocytium sp. (e.g. Ophiocytium majus or Ophiocytium parvulum), Porphyridium aerugineum, a Proteomonas sp. (e.g. Proteomonas sp. NEIS-1375 or Proteomonas sp. NIES-1005), Pseudoerythrocladia kornmanni, Rhodaphanes brevistipitata, a Rhodomonas sp. (e.g. Rhodomonas salina, Rhodomonas sp. CCMP740, Rhodomonas sp. CCMP760, Rhodomonas sp. NIES-1006, Rhodomonas sp. NIES-1730, Rhodomonas sp. NIES-2332, or Rhodomonas sp. NIES-698), Sahlingia subintegra, Storeatula sp. CCMP1868, Teleaulax amphioxea, or a Xanthonema sp. (e.g. Xanthonema bristolianum, Xanthonema cf hormidiodes, Xanthonema debile, Xanthonema exile, Xanthonema hormidioldes, Xanthonema solidum, Xanthonema sp. CCAP 836/5, Xanthonema sp. SAG 2179, Xanthonema sp. SAG 2189, Xanthonema sp. SAG 2192, or Xanthonema sp. SAG 60.94). In a further aspect, the gene that encodes RuBisCO large subunit 1 has SEQ ID NO. 2 or at least 70% homology thereto, at least 75% homology thereto, at least 80% homology thereto, at least 85% homology thereto, at least 90% homology thereto, at least 95% homology thereto, or at least 99% homology thereto.

Other sequences encoding RuBisCO large subunit 1 or related or homologous genes can be identified in a database such as, for example, GenBank. In one aspect, the gene that encodes RuBisCO large subunit 1 is isolated from Guillardia theta and can be identified by the GI number NC_000926.1 in the GenBank database. In another aspect, sequences useful herein include those with GI numbers listed in Table 2:

TABLE 2
RuBisCO Large Subunit

| Source Organism | Sequence Description | GI Number |
|---|---|---|
| Guillardia theta | Chloroplast genomic DNA | NC_000926.1 |
| Hanusia phi | RuBisCO large subunit | MG646607.1 |
| Guillardia theta | RuBisCO large subunit | MK818447.1 |
| Guillardia theta | Chloroplast genomic DNA | KT428890.1 |
| Storeatula sp. CCMP1868 | Chloroplast genomic DNA | KY856940.1 |
| Cryptomonas sp. NIES-3952 | Chloroplast genomic DNA | LC648965.1 |
| Rhodomonas salina | Chloroplast genomic DNA | NC_009573.1 |
| Cryptomonas pyrenoidifera | Chloroplast genomic DNA | NC_069042.1 |
| Rhodomonas sp. NIES-1730 | Chloroplast genomic DNA | LC648961.1 |
| Cryptomonas curvata | Chloroplast genomic DNA | LC648951.1 |
| Rhodomonas sp. NIES-698 | Chloroplast genomic DNA | LC648954.1 |
| Cryptomonas tetrapyrenoidosa | Chloroplast genomic DNA | LC648953.1 |
| Rhodomonas sp. NIES-2332 | Chloroplast genomic DNA | LC648963.1 |
| Cryptomonas curvata | Chloroplast genomic DNA | LC484192.1 |
| Teleaulax amphioxeia | Chloroplast genomic DNA | NC_027589.1 |
| Hanusia phi | RuBisCO large subunit | KX777654.1 |
| Cryptomonas curvata | Chloroplast genomic DNA | NC_035720.1 |
| Chroomonas debatzensis | Chloroplast genomic DNA | LC648962.1 |
| Chroomonas collegionis | Chloroplast genomic DNA | LC648955.1 |
| Rhodomonas sp. NIES-1006 | Chloroplast genomic DNA | LC648958.1 |
| Cryptophyta sp. CCMP2293 | Chloroplast genomic DNA | MK798155.1 |
| Hemiselmis andersenii | Chloroplast genomic DNA | LC648964.1 |
| Chroomonas placoidea | Chloroplast genomic DNA | NC_035721.1 |
| Chroomonas mesostigmatica | Chloroplast genomic DNA | KY860574.1 |
| CCMP1168 | | |
| Cryptomonas gyropyrenoidosa | Chloroplast genomic DNA | NC_077586.1 |
| Proteomonas sp. NIES-1005 | Chloroplast genomic DNA | LC648957.1 |
| Proteomonas sp. NEIS-1375 | Chloroplast genomic DNA | LC648960.1 |
| Cryptomonas sp. | RuBisCO large subunit | MG646601.1 |
| Rhodomonas sp. CCMP760 | RuBisCO large subunit | MG646616.1 |
| Rhodomonas salina | RuBisCO large subunit | MG646611.1 |
| Rhodomonas sp. | RuBisCO large subunit | MG646613.1 |
| Cryptomonas pyrenoidifera | RuBisCO large subunit | AM051217.1 |
| Rhodomonas sp. | RuBisCO large subunit | MG646614.1 |
| Rhodomonas sp. | RuBisCO large subunit | MG646612.1 |
| Cryptomonas borealis | Chloroplast genomic DNA | LC648950.1 |
| Cryptomonas marssonii | RuBisCO large subunit | AM051209.1 |
| Cryptomonas obovoidea | RuBisCO large subunit | AM051223.1 |
| Cryptomonas tetrapyrenoidosa | RuBisCO large subunit | AM051219.1 |
| Cryptomonas erosa | RuBisCO large subunit | MG646599.1 |
| Cryptomonas erosa | RuBisCO large subunit | MG646600.1 |
| Cryptomonas pyrenoidifera | RuBisCO large subunit | AM051216.1 |
| Cryptophyta sp. ECY-2019a | RuBisCO large subunit | MG646606.1 |
| Cryptomonas tetrapyrenoidosa | RuBisCO large subunit | AM051220.1 |
| Cryptomonas curvata | RuBisCO large subunit | AM051204.1 |
| Cryptomonas curvata | RuBisCO large subunit | AM051205.1 |
| Cryptomonas obovoidea | RuBisCO large subunit | AM051221.1 |
| Cryptomonas sp. CCAC 0109 | RuBisCO large subunit | AM051222.1 |
| Cryptomonas erosa | RuBisCO large subunit | MG646598.1 |
| Cryptomonas sp. NIES-1327 | Chloroplast genomic DNA | LC648959.1 |
| Cryptomonas sp. NIES-345 | Chloroplast genomic DNA | LC648952.1 |
| Rhodomonas sp. CCMP740 | RuBisCO large subunit | MG646615.1 |
| Hemiselmis tepida | RuBisCO large subunit | MG646605.1 |
| Pseudoerythrocladia kornmannii | Chloroplast genomic DNA | NC_062386.1 |
| Pseudoerythrocladia kornmannii | Chloroplast genomic DNA | MW675664.1 |
| Rhodomonas sp. | RuBisCO large subunit | MG646617.1 |
| Hemiselmis andersenii | RuBisCO large subunit | MG646603.1 |
| Cryptomonas ovata | RuBisCO large subunit | AM051210.1 |
| Botrydiopsis sp. | Photosystem II protein D1 | MK909755.1 |
| Erythrolobus coxiae | Chloroplast genomic DNA | NC_062391.1 |
| Heterothrix mucicola | Photosystem II protein D1 | EF455957.1 |
| Erythrotrichia foliiformis | Chloroplast genomic DNA | MW675678.1 |
| Cryptophyta sp. CCMP2293 | RuBisCO large subunit | MG646618.1 |
| Ophiocytium majus | RuBisCO large subunit | MK482708.1 |
| Ophiocytium parvulum | RuBisCO large subunit | MK482697.1 |
| Hemiselmis cryptochromatica | RuBisCO large subunit | MG646604.1 |
| Heterothrix mucicola | Photosystem II protein D1 | MK804169.1 |
| Xanthonema sp. SAG 60.94 | Photosystem II protein D1 | EF455977.1 |
| Botrydium granulatum | Photosystem II protein D1 | EF455980.1 |
| Botrydium stoloniferum | RuBisCO large subunit | AF465707.1 |
| Xanthonema sp. SAG 2189 | Photosystem II protein D1 | EF455954.1 |
| Porphyridium aerugineum | Chloroplast genomic DNA | NC_062300.1 |
| Xanthonema sp. SAG 2192 | Genomic DNA | EF426794.1 |
| Xanthonema cf. hormidioides | Photosystem II protein D1 | EF455953.1 |
| Porphyridium aerugineum | RuBisCO large subunit | X17597.1 |
| Cryptomonas marssonii | RuBisCO large subunit | AM051208.1 |
| Xanthonema bristolianum | Photosystem II protein D1 | MK792448.1 |
| Xanthonema bristolianum | Photosystem II protein D1 | MW176120.1 |
| Xanthonema bristolianum | Photosystem II protein D1 | MW176119.1 |
| Xanthonema bristolianum | Photosystem II protein D1 | MK804168.1 |
| Xanthonema bristolianum | Photosystem II protein D1 | MK804167.1 |
| Xanthonema bristolianum | Photosystem II protein D1 | MK804166.1 |
| Botrydium stoloniferum | Photosystem II protein D1 | EF455981.1 |
| Xanthonema sp. CCAP 836/5 | Photosystem II protein D1 | EF455940.1 |
| Xanthonema exile | Photosystem II protein D1 | EF455929.1 |
| Xanthonema bristolianum | Photosystem II protein D1 | EF455955.1 |
| Xanthonema exile | Photosystem II protein D1 | EF455937.1 |
| Ophiocytium majus | RuBisCO large subunit | MK482707.1 |
| Ophiocytium sp. | RuBisCO large subunit | MK482706.1 |
| Xanthonema solidum | Photosystem II protein D1 | EF455973.1 |
| Ophiocytium majus | Photosystem II protein D1 | EF455971.1 |
| Chrysoparadoxa australica | RuBisCO large subunit | MK189080.1 |
| Ophiocytium majus | RuBisCO large subunit | MK482705.1 |
| Sahlingia subintegra | Chloroplast genomic DNA | NC_062389.1 |
| Rhodaphanes brevistipitata | Chloroplast genomic DNA | NC_062393.1 |
| Xanthonema sp. SAG 2179 | Photosystem II protein D1 | EF426796.1 |
| Xanthonema debile | Photosystem II protein D1 | EF455975.1 |
| Xanthonema hormidioides | Photosystem II protein D1 | EF455939.1 |
| Xanthonema hormidioides | Photosystem II protein D1 | EF455922.1 |
| Bumilleriopsis sp. SAG 33.93 | Photosystem II protein D1 | EF431849.1 |
| Botrydium sp. | Photosystem II protein D1 | MW176112.1 |

In one aspect, the DNA constructs disclosed herein incorporate a gene that expresses tonB. TonB is a beta barrel protein found in the outer membrane of gram-negative bacteria. In a still further aspect, tonB proteins are involved with the uptake and/or transport of large substrates including iron siderophore complexes, heme, and other chelated forms of iron.

In one aspect, the gene that encodes tonB is isolated from a bacterium such as, for example, Pseudomonas sp. (e.g. Pseudomonas asiatica, Pseudomonas entomophila, Pseudomonas entomophila L48, Pseudomonas fakonensis, Pseudomonas fluorescens, Pseudomonas hunanensis, Pseudomonas inefficax, Pseudomonas kurunegalensis, Pseudomonas maumuensis, Pseudomonas monteilii, Pseudomonas mosselii, Pseudomonas muyukensis, Pseudomonas oryziphila, Pseudomonas plecoglossicida, Pseudomonas putida, Pseudomonas putida DOT-T1E, Pseudomonas putida GB-1, Pseudomonas putida HB3267, Pseudomonas putida NBRC 14164, Pseudomonas putida S13.1.2, Pseudomonas putida S16, Pseudomonas shirazica, Pseudomonas sichuanensis, Pseudomonas soli, Pseudomonas sp. 13159349, Pseudomonas sp. 2hn, Pseudomonas sp. A2, Pseudomonas sp. B21-023, Pseudomonas sp. B21-044, Pseudomonas sp. B03-4, Pseudomonas sp. CCOS 191, Pseudomonas sp. CFA, Pseudomonas sp. CIP-10, Pseudomonas sp. FGI182, Pseudomonas sp. HD6515, Pseudomonas sp. JY-Q, Pseudomonas sp. KU43P, Pseudomonas sp. LTGT-11-2Z, Pseudomonas sp. NBB, Pseudomonas sp. PONIH3, Pseudomonas sp. RC3H12, Pseudomonas sp. RtIB026, Pseudomonas sp. XWY-1, Pseudomonas xantholysinigenes, or Pseudomonas xanthosomatis). In a further aspect, the gene that encodes tonB has SEQ ID NO. 3 or at least 70% homology thereto, at least 75% homology thereto, at least 80% homology thereto, at least 85% homology thereto, at least 90% homology thereto, at least 95% homology thereto, or at least 99% homology thereto.

Other sequences encoding tonB or related or homologous genes can be identified in a database such as, for example, GenBank. In one aspect, the gene that encodes tonB is isolated from Pseudomonas entomophila and can be identified by the GI number CP132921.1 in the GenBank database. In another aspect, sequences useful herein include those with GI numbers listed in Table 3:

TABLE 3
TonB

| Source Organism | Sequence Description | GI Number |
|---|---|---|
| Pseudomonas entomophila | Genomic DNA | CP132921.1 |
| Pseudomonas entomophila L48 | Genomic DNA | CT573326.1 |
| Pseudomonas entomophila | Genomic DNA | CP070982.1 |
| Pseudomonas entomophila | Genomic DNA | CP063832.1 |
| Pseudomonas xantholysinigenes | Genomic DNA | CP077095.1 |
| Pseudomonas maumuensis | Genomic DNA | CP077077.1 |
| Pseudomonas fakonensis | Genomic DNA | CP077076.1 |
| Pseudomonas sp. CCOS 191 | Genomic DNA | LN847264.1 |
| Pseudomonas sichuanensis | Genomic DNA | CP087165.1 |
| Pseudomonas oryziphila | Genomic DNA | CP034337.1 |
| Pseudomonas oryziphila | Genomic DNA | CP034338.1 |
| Pseudomonas sp. 2hn | Genomic DNA | CP081016.1 |
| Pseudomonas xanthosomatis | Genomic DNA | CP077075.1 |
| Pseudomonas mosselii | Genomic DNA | CP103054.1 |
| Pseudomonas sichuanensis | Genomic DNA | CP087185.1 |
| Pseudomonas plecoglossicida | Genomic DNA | CP031146.1 |
| Pseudomonas mosselii | Genomic DNA | CP024159.1 |
| Pseudomonas mosselii | Genomic DNA | CP023299.1 |
| Pseudomonas mosselii | Genomic DNA | CP133092.1 |
| Pseudomonas mosselii | Genomic DNA | CP128544.1 |
| Pseudomonas mosselii | Genomic DNA | CP104107.1 |
| Pseudomonas mosselii | Genomic DNA | CP095556.1 |
| Pseudomonas plecoglossicida | Genomic DNA | CP050291.1 |
| Pseudomonas putida | Genomic DNA | CP014343.1 |
| Pseudomonas sp. RtlB026 | Genomic DNA | AP023348.2 |
| Pseudomonas mosselii | Genomic DNA | CP081966.1 |
| Pseudomonas muyukensis | Genomic DNA | CP077073.1 |
| Pseudomonas soli | Genomic DNA | CP083803.1 |
| Pseudomonas sp. PONIH3 | Genomic DNA | CP026386.1 |
| Pseudomonas soli | Genomic DNA | CP128543.1 |
| Pseudomonas sp. B21-023 | Genomic DNA | CP087190.1 |
| Pseudomonas sp. B21-044 | Genomic DNA | CP087172.1 |
| Pseudomonas soli | Genomic DNA | CP009365.1 |
| Pseudomonas sp. RC3H12 | Genomic DNA | CP075595.1 |
| Pseudomonas putida | Genomic DNA | CP018846.1 |
| Pseudomonas putida S13.1.2 | Genomic DNA | CP010979.1 |
| Pseudomonas fluorescens | Genomic DNA | CP100660.1 |
| Pseudomonas putida | Genomic DNA | AP022324.1 |
| Pseudomonas monteilii | Genomic DNA | CP040324.1 |
| Pseudomonas sp. LTGT-11-2Z | Genomic DNA | CP033104.1 |
| Pseudomonas monteilii | Genomic DNA | CP022562.1 |
| Pseudomonas putida | Genomic DNA | CP018743.1 |
| Pseudomonas monteilii | Genomic DNA | CP128547.1 |
| Pseudomonas monteilii | Genomic DNA | CP128546.1 |
| Pseudomonas monteilii | Genomic DNA | CP128541.1 |
| Pseudomonas monteilii | Genomic DNA | CP128545.1 |
| Pseudomonas monteilii | Genomic DNA | CP128549.1 |
| Pseudomonas shirazica | Genomic DNA | CP075569.1 |
| Pseudomonas monteilii | Genomic DNA | CP060595.1 |
| Pseudomonas putida | Genomic DNA | CP124529.1 |
| Pseudomonas asiatica | Genomic DNA | CP127872.1 |
| Pseudomonas putida | Genomic DNA | CP026115.2 |
| Pseudomonas sp. KU43P | Genomic DNA | AP019365.1 |
| Pseudomonas sp. 13159349 | Genomic DNA | CP045553.1 |
| Pseudomonas inefficax | Genomic DNA | CP134401.1 |
| Pseudomonas shirazica | Genomic DNA | CP127845.1 |
| Pseudomonas asiatica | Genomic DNA | CP128542.1 |
| Pseudomonas asiatica | Genomic DNA | CP107576.1 |
| Pseudomonas asiatica | Genomic DNA | CP101700.1 |
| Pseudomonas putida HB3267 | Genomic DNA | CP003738.1 |
| Pseudomonas shirazica | Genomic DNA | CP063456.1 |
| Pseudomonas asiatica | Genomic DNA | CP061848.1 |
| Pseudomonas asiatica | Genomic DNA | CP061335.1 |
| Pseudomonas putida | Genomic DNA | CP050951.1 |
| Pseudomonas sp. | Genomic DNA | CP119325.1 |
| Pseudomonas putida | Genomic DNA | CP096581.1 |
| Pseudomonas monteilii | Genomic DNA | CP014062.1 |
| Pseudomonas putida | Genomic DNA | LR813085.1 |
| Pseudomonas putida | Genomic DNA | LR813083.1 |
| Pseudomonas putida | Genomic DNA | CP137621.1 |
| Pseudomonas hunanensis | Genomic DNA | OY781045.1 |
| Pseudomonas putida | Genomic DNA | CP101910.1 |
| Pseudomonas putida | Genomic DNA | CP069080.1 |
| Pseudomonas sp. CIP-10 | Genomic DNA | CP087160.1 |
| Pseudomonas putida NBRC 14164 | Genomic DNA | AP013070.1 |
| Pseudomonas putida | Genomic DNA | CP046872.1 |
| Pseudomonas monteilii | Genomic DNA | CP043396.1 |
| Pseudomonas monteilii | Genomic DNA | CP043395.1 |
| Pseudomonas sp. NBB | Genomic DNA | CP103994.1 |
| Pseudomonas kurunegalensis | Genomic DNA | CP128548.1 |
| Pseudomonas asiatica | Genomic DNA | CP128558.1 |
| Pseudomonas asiatica | Genomic DNA | CP084714.1 |
| Pseudomonas putida | Genomic DNA | CP109606.1 |
| Pseudomonas sp. A2 | Genomic DNA | CP039127.1 |
| Pseudomonas sp. FGI182 | Genomic DNA | CP007012.1 |
| Pseudomonas putida S16 | Genomic DNA | CP002870.1 |
| Pseudomonas putida GB-1 | Genomic DNA | CP000926.1 |
| Pseudomonas putida | Genomic DNA | CP045551.1 |
| Pseudomonas sp. XWY-1 | Genomic DNA | CP026332.1 |
| Pseudomonas sp. BO3-4 | Genomic DNA | CP139018.1 |
| Pseudomonas hunanensis | Genomic DNA | CP131127.1 |
| Pseudomonas sp. JY-Q | Genomic DNA | CP011525.1 |
| Pseudomonas putida | Genomic DNA | CP120969.1 |
| Pseudomonas putida | Genomic DNA | CP096920.1 |
| Pseudomonas putida DOT-T1E | Genomic DNA | CP110782.1 |
| Pseudomonas sp. HD6515 | Genomic DNA | CP079827.1 |
| Pseudomonas putida | Genomic DNA | CP047311.1 |
| Pseudomonas putida DOT-T1E | Genomic DNA | CP003734.1 |
| Pseudomonas sp. CFA | Genomic DNA | CP044546.1 |
| Pseudomonas putida | Genomic DNA | CP043576.1 |

In one aspect, the DNA constructs disclosed herein incorporate a gene that encodes phosphoenol pyruvate carboxykinase. In a further aspect, phosphoenol pyruvate carboxykinase is an enzyme used in the gluconeogenesis metabolic pathway. Phosphoenol pyruvate carboxykinase in the cytosol converts oxaloacetate into phosphoenol pyruvate and carbon dioxide in the presence of GTP in an irreversible reaction. Mitochondrial isoforms also exist and can be transported to the cytosol. Plants and bacteria also encode phosphoenol pyruvate carboxykinase enzymes.

In one aspect, the gene that encodes phosphoenol pyruvate carboxykinase is isolated from a mammal such as, for example, human, chimpanzee, bonobo, Western lowland gorilla, siamang, Bornean orangutan, Sumatran orangutan, Northern white-cheeked gibbon, silvery gibbon, Indochinese rhesus macaque, Southern pig-tailed macaque, green monkey, drill, Tibetan macaque, crab-eating macaque, Ugandan red colobus, olive baboon, sooty mangabey, gelada, Angola colobus, Francois' langur, black-and-white snub-nosed monkey, golden snub-nosed monkey, groundhog, yellow-bellied marmot, Alpine marmot, Nancy Ma's night monkey, tufted capuchin, Panamanian white-faced capuchin, Northern greater galago, American pika, greater spear-nosed bat, common marmoset, Ryukyu mouse, Arctic ground squirrel, Eastern gray squirrel, plateau pika, Gairdner's shrewmouse, common vampire bat, house mouse, Parnell's mustached bat, thirteen-lined ground squirrel, pale spear-nosed bat, fat sand rat, black-capped squirrel monkey, guinea pig, long-tailed chinchilla, Mongolian gerbil. common degu, black rat, cape elephant shrew, great roundleaf bat, Sturnira hondurensis, gray mouse lemur, American beaver. cactus mouse, brown rat. lesser long-nosed bat. African woodland thicket rat. South-central black rhinoceros. Southern white rhinoceros, velvety free-tailed bat, African grass rat, grey seal, natal long-fingered bat, vaquita, California deermouse, Southern grasshopper mouse, wood mouse, Philippine tarsier, banner-tailed kangaroo rat, common bent-wing bat, naked mole-rat, long-finned pilot whale, white-beaked dolphin, nine-banded armadillo, Sunda slow loris, or ring-tailed lemur. In a further aspect, the gene that encodes phosphoenol pyruvate carboxykinase has SEQ ID NO. 4 or at least 70% homology thereto, at least 75% homology thereto, at least 80% homology thereto, at least 85% homology thereto, at least 90% homology thereto, at least 95% homology thereto, or at least 99% homology thereto.

Other sequences encoding phosphoenol pyruvate carboxykinase or related or homologous genes can be identified in a database such as, for example, GenBank. In one aspect, the gene that encodes phosphoenol pyruvate carboxykinase is isolated from Homo sapiens and can be identified by the GI number NM_002591.4 in the GenBank database. In another aspect, sequences useful herein include those with GI numbers listed in Table 4:

TABLE 4
Phosphoenol Pyruvate Carboxykinase

| Source Organism | Sequence Description | GI Number |
|---|---|---|
| Homo sapiens | Phosphoenolpyruvate carboxykinase 1 | NM_002591.4 |
| Homo sapiens | Similar to phosphoenolpyruvate | AK290802.1 |
| | carboxykinase 1 | |
| synthetic construct | Phosphoenolpyruvate carboxykinase 1 | DQ896497.2 |
| synthetic construct | Phosphoenolpyruvate carboxykinase 1 | DQ893518.2 |
| Homo sapiens | Phosphoenolpyruvate carboxykinase 1 | BC023978.1 |
| synthetic construct | Phosphoenolpyruvate carboxykinase 1 | AY888078.1 |
| Pan troglodytes | Phosphoenolpyruvate carboxykinase 1 | XM_514745.8 |
| Homo sapiens | Phosphoenolpyruvate carboxykinase 1 | L05144.1 |
| Pan paniscus | Phosphoenolpyruvate carboxykinase 1 | XM_003806166.5 |
| Gorilla gorilla gorilla | Phosphoenolpyruvate carboxykinase 1 | XM_004062425.4 |
| Symphalangus syndactylus | Phosphoenolpyruvate carboxykinase 1 | XM_055265768.1 |
| Pongo pygmaeus | Phosphoenolpyruvate carboxykinase 1 | XM_054468194.1 |
| Pongo abelii | Phosphoenolpyruvate carboxykinase 1 | NM_001133286.1 |
| Nomascus leucogenys | Phosphoenolpyruvate carboxykinase 1 | XM_030826487.1 |
| Hylobates moloch | Phosphoenolpyruvate carboxykinase 1 | XM_032142591.2 |
| Macaca mulatta | Phosphoenolpyruvate carboxykinase 1 | XM_001086710.4 |
| Macaca nemestrina | Phosphoenolpyruvate carboxykinase 1 | XM_011722760.2 |
| Chlorocebus sabaeus | Phosphoenolpyruvate carboxykinase 1 | XM_008013224.2 |
| Mandrillus leucophaeus | Phosphoenolpyruvate carboxykinase 1 | XM_011978164.1 |
| Macaca thibetana thibetana | Phosphoenolpyruvate carboxykinase 1 | XM_050806873.1 |
| Macaca fascicularis | Phosphoenolpyruvate carboxykinase 1 | XM_005569429.3 |
| Piliocolobus tephrosceles | Phosphoenolpyruvate carboxykinase 1 | XM_023231940.2 |
| Papio anubis | Phosphoenolpyruvate carboxykinase 1 | XM_003904570.4 |
| Cercocebus atys | Phosphoenolpyruvate carboxykinase 1 | XM_012058916.1 |
| Theropithecus gelada | Phosphoenolpyruvate carboxykinase 1 | XM_025399021.1 |
| Colobus angolensis palliatus | Phosphoenolpyruvate carboxykinase 1 | XM_011936682.1 |
| Trachypithecus francoisi | Phosphoenolpyruvate carboxykinase 1 | XM_033186833.1 |
| Rhinopithecus bieti | Phosphoenolpyruvate carboxykinase 1 | XM_017852132.1 |
| Rhinopithecus roxellana | Phosphoenolpyruvate carboxykinase 1 | XM_010365119.1 |
| Marmota monax | Phosphoenolpyruvate carboxykinase 1 | XM_058575417.1 |
| Marmota monax | Phosphoenolpyruvate carboxykinase 1 | XM_058575416.1 |
| Marmota flaviventris | Phosphoenolpyruvate carboxykinase 1 | XM_027946450.1 |
| Marmota marmota marmota | Phosphoenolpyruvate carboxykinase 1 | XM_048810871.1 |
| Marmota marmota marmota | Phosphoenolpyruvate carboxykinase 1 | XM_015479925.2 |
| Aotus nancymaae | Phosphoenolpyruvate carboxykinase 1 | XM_012467511.2 |
| Sapajus apella | Phosphoenolpyruvate carboxykinase 1 | XM_032257206.1 |
| Pteronotus parnellii | Phosphoenolpyruvate carboxykinase 1 | XM_054573573.1 |
| mesoamericanus | | |
| Cebus imitator | Phosphoenolpyruvate carboxykinase 1 | XM_017536565.2 |
| Otolemur garnettii | Phosphoenolpyruvate carboxykinase 1 | XM_003787726.2 |
| Ochotona princeps | Phosphoenolpyruvate carboxykinase 1 | XM_058679364.1 |
| Ochotona princeps | Phosphoenolpyruvate carboxykinase 1 | XM_058679363.1 |
| Ochotona princeps | Phosphoenolpyruvate carboxykinase 1 | XM_004586104.3 |
| Phyllostomus hastatus | Phosphoenolpyruvate carboxykinase 1 | XM_045833174.1 |
| Callithrix jacchus | Phosphoenolpyruvate carboxykinase 1 | XM_002747710.6 |
| Mus caroli | Phosphoenolpyruvate carboxykinase 1 | XM_021149509.2 |
| Urocitellus parryii | Phosphoenolpyruvate carboxykinase 1 | XM_026406798.1 |
| Sciurus carolinensis | Phosphoenolpyruvate carboxykinase 1 | XM_047534453.1 |
| Ochotona curzoniae | Phosphoenolpyruvate carboxykinase 1 | XM_040988140.1 |
| Mus pahari | Phosphoenolpyruvate carboxykinase 1 | XM_021194801.2 |
| Desmodus rotundus | Phosphoenolpyruvate carboxykinase 1 | XM_024559406.3 |
| Mus musculus | Phosphoenolpyruvate carboxykinase 1 | NM_011044.3 |
| Mus musculus | Phosphoenolpyruvate carboxykinase 1 | AK133496.1 |
| Mus musculus | Phosphoenolpyruvate carboxykinase 1 | AK028046.1 |
| Mus musculus | Phosphoenolpyruvate carboxykinase 1 | AK030327.1 |
| Pteronotus parnellii | Phosphoenolpyruvate carboxykinase 1 | KJ957756.1 |
| Ictidomys tridecemlineatus | Phosphoenolpyruvate carboxykinase 1 | XM_013359516.3 |
| Ictidomys tridecemlineatus | Phosphoenolpyruvate carboxykinase 1 | XM_040275463.1 |
| Ictidomys tridecemlineatus | Phosphoenolpyruvate carboxykinase 1 | XM_005327077.3 |
| Mus musculus | Phosphoenolpyruvate carboxykinase 1 | BC037629.1 |
| Mus musculus | Phosphoenolpyruvate carboxykinase 1 | AK149525.1 |
| Phyllostomus discolor | Phosphoenolpyruvate carboxykinase 1 | XM_028524066.2 |
| Psammomys obesus | Phosphoenolpyruvate carboxykinase 1 | XM_055596263.1 |
| Saimiri boliviensis boliviensis | Phosphoenolpyruvate carboxykinase 1 | XM_003932619.3 |
| Cavia porcellus | Phosphoenolpyruvate carboxykinase 1 | XM_003463639.3 |
| Chinchilla lanigera | Phosphoenolpyruvate carboxykinase 1 | XM_005392250.1 |
| Meriones unguiculatus | Phosphoenolpyruvate carboxykinase 1 | XM_060382768.1 |
| Meriones unguiculatus | Phosphoenolpyruvate carboxykinase 1 | XM_021657013.2 |
| Octodon degus | Phosphoenolpyruvate carboxykinase 1 | XM_004636004.2 |
| Rattus rattus | Phosphoenolpyruvate carboxykinase 1 | XM_032904709.1 |
| Elephantulus edwardii | Phosphoenolpyruvate carboxykinase 1 | XM_006896853.1 |
| Hipposideros armiger | Phosphoenolpyruvate carboxykinase 1 | XM_019637125.1 |
| Sturnira hondurensis | Phosphoenolpyruvate carboxykinase 1 | XM_037059963.1 |
| Microcebus murinus | Phosphoenolpyruvate carboxykinase 1 | XM_012770477.2 |
| Castor canadensis | Phosphoenolpyruvate carboxykinase 1 | XM_020152656.1 |
| Peromyscus eremicus | Phosphoenolpyruvate carboxykinase 1 | XM_059259789.1 |
| Rattus norvegicus | Phosphoenolpyruvate carboxykinase 1 | BC081900.1 |
| Rattus norvegicus | Phosphoenolpyruvate carboxykinase 1 | NM_198780.3 |
| Leptonycteris yerbabuenae | Phosphoenolpyruvate carboxykinase 1 | KJ957758.1 |
| Grammomys surdaster | Phosphoenolpyruvate carboxykinase 1 | XM_028769739.1 |
| Diceros bicornis minor | Phosphoenolpyruvate carboxykinase 1 | XM_058562094.1 |
| Ceratotherium simum simum | Phosphoenolpyruvate carboxykinase 1 | XM_004430240.2 |
| Molossus molossus | Phosphoenolpyruvate carboxykinase 1 | XM_036245504.1 |
| Arvicanthis niloticus | Phosphoenolpyruvate carboxykinase 1 | XM_034495255.1 |
| Ochotona curzoniae | Phosphoenolpyruvate carboxykinase 1 | XM_040988141.1 |
| Halichoerus grypus | Phosphoenolpyruvate carboxykinase 1 | XM_036094401.1 |
| Miniopterus natalensis | Phosphoenolpyruvate carboxykinase 1 | XM_016203393.1 |
| Phocoena sinus | Phosphoenolpyruvate carboxykinase 1 | XM_032606926.1 |
| Elephantulus edwardii | Phosphoenolpyruvate carboxykinase 1 | XM_006896852.1 |
| Peromyscus californicus | Phosphoenolpyruvate carboxykinase 1 | XM_052725116.1 |
| insignis | | |
| Onychomys torridus | Phosphoenolpyruvate carboxykinase 1 | XM_036186539.1 |
| Apodemus sylvaticus | Phosphoenolpyruvate carboxykinase 1 | XM_052181590.1 |
| Carlito syrichta | Phosphoenolpyruvate carboxykinase 1 | XM_008054412.1 |
| Dipodomys spectabilis | Phosphoenolpyruvate carboxykinase 1 | XM_042699217.1 |
| Miniopterus schreibersii | Phosphoenolpyruvate carboxykinase 1 | KJ957759.1 |
| Heterocephalus glaber | Phosphoenolpyruvate carboxykinase 1 | XM_004840930.3 |
| Globicephala melas | Phosphoenolpyruvate carboxykinase 1 | XM_030841614.2 |
| Lagenorhynchus albirostris | Phosphoenolpyruvate carboxykinase 1 | XM_060123918.1 |
| Dasypus novemcinctus | Phosphoenolpyruvate carboxykinase 1 | XM_004468027.4 |
| Nycticebus coucang | Phosphoenolpyruvate carboxykinase 1 | XM_053574511.1 |
| Lemur catta | Phosphoenolpyruvate carboxykinase 1 | XM_045528475.1 |

In one aspect, the DNA constructs disclosed herein incorporate a gene that encodes phosphoenolpyruvate carboxylase. In a further aspect, phosphoenolpyruvate carboxylase is an enzyme that catalyzes the addition of bicarbonate to phosphoenolpyruvate to form oxaloacetate; inorganic phosphate is also a product of this reaction. Phosphoenolpyruvate carboxylase is used for carbon fixation in C₄ plants, crassulacean acid metabolism plants, and certain bacteria.

In one aspect, the gene that encodes phosphoenolpyruvate carboxylase is isolated from a plant such as, for example, rapeseed, thale cress, wild cabbage, turnip, leaf mustard, camelina, radish, pink shepherd's purse, salt cress, saltwater cress, giant spider flower, upland cotton, Peruvian cotton, tree cotton, common hibiscus, crookneck pumpkin, cacao, riverbank grape, jujube, malletwood tree, mango, sweet orange, bitter melon, clementine, common grape vine, Herrania umbratical, summer squash, Mulberry, Oryza brachyantha, Chinese white pear, blue lilly pilly, or flooded gum tree. In a further aspect, the gene that encodes phosphoenolpyruvate carboxylase has SEQ ID NO. 5 or at least 70% homology thereto, at least 75% homology thereto, at least 80% homology thereto, at least 85% homology thereto, at least 90% homology thereto, at least 95% homology thereto, or at least 99% homology thereto.

Other sequences encoding phosphoenolpyruvate carboxylase or related or homologous genes can be identified in a database such as, for example, GenBank. In one aspect, the gene that encodes phosphoenolpyruvate carboxylase is isolated from Brassica napus and can be identified by the GI number XM_048750595 in the GenBank database. In another aspect, sequences useful herein include those with GI numbers listed in Table 5:

TABLE 5
Phosphoenolpyruvate Carboxylase 1

| Source Organism | Sequence Description | GI Number |
|---|---|---|
| Brassica napus | Phosphoenolpyruvate carboxylase 1 | XM_048750595.1 |
| Brassica napus | Phosphoenolpyruvate carboxylase 1 | XM_048750594.1 |
| Brassica napus | Phosphoenolpyruvate carboxylase 1 | XM_048750593.1 |
| Brassica napus | Phosphoenolpyruvate carboxylase 1 | XM_048750592.1 |
| Brassica napus | Phosphoenolpyruvate carboxylase 1 | XM_048750591.1 |
| Brassica napus | Phosphoenolpyruvate carboxylase 1 | XM_048750590.1 |
| Brassica napus | Phosphoenolpyruvate carboxylase 1 | XM_013800011.3 |
| Brassica napus | Phosphoenolpyruvate carboxylase 1 | XM_048750588.1 |
| Arabidopsis thaliana | Phosphoenolpyruvate carboxylase 1 | NM_001036102.3 |
| Arabidopsis thaliana | Phosphoenolpyruvate carboxylase 1 | NM_001036101.2 |
| Arabidopsis thaliana | Phosphoenolpyruvate carboxylase 1 | NM_104209.3 |
| Arabidopsis thaliana | Phosphoenolpyruvate carboxylase 1 | AJ532901.1 |
| Arabidopsis thaliana | mRNA | AY057507.1 |
| Arabidopsis thaliana | mRNA | AK317277.1 |
| Brassica oleracea var. | Phosphoenolpyruvate carboxylase 1 | XM_013773407.1 |
| oleracea | | |
| Arabidopsis lyrata subsp. | Phosphoenolpyruvate carboxylase 1 | XM_021011473.1 |
| lyrata | | |
| Arabidopsis lyrata subsp. | Phosphoenolpyruvate carboxylase 1 | XM_021011472.1 |
| lyrata | | |
| Brassica rapa | Phosphoenolpyruvate carboxylase 1 | XM_009108735.3 |
| Brassica napus | Phosphoenolpyruvate carboxylase 1 | XM_013817493.3 |
| Brassica juncea | Phosphoenolpyruvate carboxylase 1 | AJ223497.1 |
| Camelina sativa | Phosphoenolpyruvate carboxylase 1 | XM_019239140.1 |
| Raphanus sativus | Phosphoenolpyruvate carboxylase 1 | XM_057000440.1 |
| Camelina sativa | Phosphoenolpyruvate carboxylase 1 | XM_010481584.1 |
| Brassica juncea | Phosphoenolpyruvate carboxylase 1 | AJ223496.1 |
| Capsella rubella | Phosphoenolpyruvate carboxylase 1 | XM_006306616.2 |
| Capsella rubella | Phosphoenolpyruvate carboxylase 1 | XM_006306617.2 |
| Camelina sativa | Phosphoenolpyruvate carboxylase 1 | XM_010463911.2 |
| Camelina sativa | Phosphoenolpyruvate carboxylase 1 | XM_010463909.2 |
| Camelina sativa | Phosphoenolpyruvate carboxylase 1 | XM_010463910.2 |
| Camelina sativa | Phosphoenolpyruvate carboxylase 1-like | XM_010502669.2 |
| Camelina sativa | Phosphoenolpyruvate carboxylase 1-like | XM_010502668.2 |
| Eutrema halophilum | mRNA | AK353394.1 |
| Brassica napus | Phosphoenolpyruvate carboxylase 1-like | XM_048760693.1 |
| Brassica napus | Phosphoenolpyruvate carboxylase 1-like | XM_048760692.1 |
| Eutrema salsugineum | Phosphoenolpyruvate carboxylase 1 | XM_006392739.2 |
| Eutrema salsugineum | Phosphoenolpyruvate carboxylase 1 | XM_024150185.1 |
| Brassica juncea | Phosphoenolpyruvate carboxylase 1 | KX352389.1 |
| Tarenaya hassleriana | Phosphoenolpyruvate carboxylase 3 | XM_010553816.2 |
| Tarenaya hassleriana | Phosphoenolpyruvate carboxylase 3 | XM_010553815.2 |
| Tarenaya hassleriana | Phosphoenolpyruvate carboxylase 1-like | XM_010544006.1 |
| Tarenaya hassleriana | Phosphoenolpyruvate carboxylase 1-like | XM_010529078.2 |
| Tarenaya hassleriana | Phosphoenolpyruvate carboxylase 1-like | XM_019201455.1 |
| Brassica napus | Phosphoenolpyruvate carboxylase 3 | XM_013880828.3 |
| Brassica napus | Phosphoenolpyruvate carboxylase 3 | XM_013880829.3 |
| Eutrema salsugineum | Phosphoenolpyruvate carboxylase 3 | XM_006406967.2 |
| Capsella rubella | Phosphoenolpyruvate carboxylase 3 | XM_006296845.2 |
| Capsella rubella | Phosphoenolpyruvate carboxylase 3 | XM_023786107.1 |
| Eutrema halophilum | mRNA | AK353350.1 |
| Camelina sativa | Phosphoenolpyruvate carboxylase 3 | XM_019246348.1 |
| Camelina sativa | Phosphoenolpyruvate carboxylase 3-like | XM_010467124.1 |
| Gossypium hirsutum | Phosphoenolpyruvate carboxylase | XM_041086879.1 |
| Gossypium raimondii | Phosphoenolpyruvate carboxylase | XM_012601897.2 |
| Arabidopsis lyrata subsp. | Phosphoenolpyruvate carboxylase 3 | XM_002882858.2 |
| lyrata | | |
| Gossypium arboreum | Phosphoenolpyruvate carboxylase | XM_017786535.2 |
| Gossypium hirsutum | Phosphoenolpyruvate carboxylase | XM_041075041.1 |
| Brassica rapa | Phosphoenolpyruvate carboxylase 3 | XM_009137165.3 |
| Hibiscus syriacus | Phosphoenolpyruvate carboxylase | XM_039166986.1 |
| Brassica rapa | Phosphoenolpyruvate carboxylase 3 | XM_033292762.1 |
| Brassica rapa | Phosphoenolpyruvate carboxylase 3 | XM_009148057.3 |
| Brassica rapa | Phosphoenolpyruvate carboxylase 3 | XM_009148056.2 |
| Brassica napus | Phosphoenolpyruvate carboxylase 3-like | XM_013838397.3 |
| Arabidopsis thaliana | Phosphoenolpyruvate carboxylase 3 | NM_112356.4 |
| Arabidopsis thaliana | Phosphoenolpyruvate carboxylase 3 | NM_001338141.1 |
| Arabidopsis thaliana | Phosphoenolpyruvate carboxylase | AF071788.1 |
| Hibiscus syriacus | Phosphoenolpyruvate carboxylase-like | XM_039145669.1 |
| Cucurbita moschata | Phosphoenolpyruvate carboxylase | XM_023084601.1 |
| Cucurbita moschata | Phosphoenolpyruvate carboxylase | XM_023084600.1 |
| Cucurbita moschata | Phosphoenolpyruvate carboxylase | XM_023084599.1 |
| Brassica oleracea var. | Phosphoenolpyruvate carboxylase 3 | XM_013782833.1 |
| oleracea | | |
| Theobroma cacao | Phosphoenolpyruvate carboxylase | XM_007021667.2 |
| Theobroma cacao | Phosphoenolpyruvate carboxylase | XM_007021666.2 |
| Arabidopsis thaliana | Phosphoenolpyruvate carboxylase | AK227556.1 |
| Arabidopsis thaliana | Genomic DNA | BT004642.1 |
| Gossypium hirsutum | Phosphoenolpyruvate carboxylase | EU032328.1 |
| Brassica napus | Phosphoenolpyruvate carboxylase 3-like | XM_048779837.1 |
| Brassica napus | Phosphoenolpyruvate carboxylase 3-like | XM_048779836.1 |
| Raphanus sativus | Phosphoenolpyruvate carboxylase 3 | XM_056985607.1 |
| Vitis riparia | Phosphoenolpyruvate carboxylase | XM_034822307.1 |
| Vitis riparia | Phosphoenolpyruvate carboxylase | XM_034822306.1 |
| Ziziphus jujuba | Phosphoenolpyruvate carboxylase | XM_016043691.4 |
| Cucurbita moschata | Phosphoenolpyruvate carboxylase | XM_023080896.1 |
| Rhodamnia argentea | Phosphoenolpyruvate carboxylase | XM_030694355.2 |
| Mangifera indica | Phosphoenolpyruvate carboxylase | XM_044617504.1 |
| Citrus sinensis | Phosphoenolpyruvate carboxylase | XM_006487390.4 |
| Citrus sinensis | Phosphoenolpyruvate carboxylase | XM_006487389.3 |
| Momordica charantia | Phosphoenolpyruvate carboxylase | XM_022303837.1 |
| Momordica charantia | Phosphoenolpyruvate carboxylase | XM_022303828.1 |
| Citrus × clementina | Phosphoenolpyruvate carboxylase | XM_024187754.1 |
| Citrus × clementina | Phosphoenolpyruvate carboxylase | XM_006442451.2 |
| Vitis vinifera | Phosphoenolpyruvate carboxylase | XM_002285405.4 |
| Herrania umbratica | Phosphoenolpyruvate carboxylase | XM_021431888.1 |
| Cucurbita pepo subsp. pepo | Phosphoenolpyruvate carboxylase | XM_023668092.1 |
| Cucurbita pepo subsp. pepo | Phosphoenolpyruvate carboxylase | XM_023668091.1 |
| Cucurbita pepo subsp. pepo | Phosphoenolpyruvate carboxylase | XM_023668090.1 |
| Cucurbita pepo subsp. pepo | Phosphoenolpyruvate carboxylase | XM_023679479.1 |
| Morus notabilis | Phosphoenolpyruvate carboxylase | XM_024173393.1 |
| Oryza brachyantha | Phosphoenolpyruvate carboxylase 2-like | XM_006659256.2 |
| Pyrus × bretschneideri | Phosphoenolpyruvate carboxylase | XM_009341762.3 |
| Syzygium oleosum | Phosphoenolpyruvate carboxylase | XM_030605022.2 |
| Eucalyptus grandis | Phosphoenolpyruvate carboxylase | XM_010062388.3 |

In one aspect, the DNA constructs disclosed herein incorporate a gene that expresses hydrogenase. Hydrogenase is a protein that catalyzes the reversible oxidation of molecular hydrogen. Numerous hydrogenases are recognized including [NiFe]hydrogenases, [NiFeSe]hydrogenases, [FeFe]hydrogenases, and [Fe]-only hydrogenases, where the chemical symbols in brackets indicate the metal ions at the catalytic centers of the protein.

In one aspect, the gene that encodes hydrogenase is isolated from a bacterium such as, for example, Acidiferrobacter sp. SPIII_3; an Acidithiobacillus species such as, for example, Acidithiobacillus caldus (e.g. strain Acidithiobacillus caldus ATCC 51756 or Acidithiobacillus caldus SM-1), Acidithiobacillus ferridurans, Acidithiobacillus ferriphilus, Acidithiobacillus ferrivorans (e.g. strain Acidithiobacillus ferrivorans SS3, Acidithiobacillus ferrooxidans (e.g. strain Acidithiobacillus ferrooxidans ATCC 23270 or Acidithiobacillus ferrooxidans ATCC 53993), Acidithiobacillus sp. ‘AMD consortium’, Acidithiobacillus sp. AMEEHan, Acidithiobacillus sp. YTS05, or Acidithiobacillus thiooxidans ATCC 19377; Aromatoleum petrolei; an Azoarcus species such as, for example, Azoarcus sp. DN11 or Azoarcus sp. KH32C; a Burkholderia species such as, for example, Burkholderia humptydooensis, Burkholderia sp. 2002721687, Burkholderia sp. HB1, or Burkholderia thailandensis (e.g. strain Burkholderia thailandensis MSMB121); a Burkholderiaceae bacterium; a Burkholderiales bacterium such as, for example, Burkholderiales bacterium GJ-E10; Candidatus Accumulibacter similis; Candidatus Desulfobacillus denitrificans; Candidatus Symbiobacter mobilis CR; a Cupriavidus species such as, for example, Cupriavidus metallidurans (e.g. strain Cupriavidus metallidurans CH34), Cupriavidus necator (e.g. strain Cupriavidus necator H16 or Cupriavidus necator N-1), Cupriavidus oxalaticus, Cupriavidus sp. KK10, or Cupriavidus sp. WKF15; Dechloromonas sp. A34; Denitratisoma oestradiolicum; Gallionella capsiferriformans ES-2; Methylococcus geothermalis; a Paraburkholderia species such as, for example, Paraburkholderia aromaticivorans, Paraburkholderia caribensis, Paraburkholderia caribensis MBA4, Paraburkholderia dioscoreae, Paraburkholderia dokdonensis, Paraburkholderia hospital, Paraburkholderia pallida, Paraburkholderia phymatum STM815, Paraburkholderia sp. PGU19, Paraburkholderia terrae, Paraburkholderia terricola, or Paraburkholderia xenovorans LB400; Paucibacter sp. S2-9; a Polaromonas species such as, for example, Polaromonas sp. JS666; Polaromonas sp. P1(28)-13, Polaromonas sp. P1(28)-8, Polaromonas sp. P1-6, or Polaromonas sp. P2-4; a Ralstonia species such as, for example, Ralstonia pickettii DTP0602 or Ralstonia syzygii R24; Rhodanobacteraceae bacterium; Rhodoferax ferrireducens (e.g. strain Rhodoferax ferrireducens T118); Rugosibacter aromaticivorans; Sideroxyarcus emersonii; a Sideroxydans species such as, for example, Sideroxydans lithotrophicus ES-1 or Sideroxydans sp. L21; Sulfuricella denitrificans skB26; Sulfuriferula plumbiphila; Sulfurimicrobium acus; Sulfuritalea hydrogenivorans sk43H; Thiomonas arsenitoxydans; a Varivoraxspecies such as, for example, Variovoraxparadoxus B4, Variovorax sp. HW608, Variovorax sp. PBL-E5, or Variovorax sp. PBL-H6; or an uncultured bacterium. In a further aspect, the gene that encodes hydrogenase has SEQ ID NO. 6 or at least 70% homology thereto, at least 75% homology thereto, at least 80% homology thereto, at least 85% homology thereto, at least 90% homology thereto, at least 95% homology thereto, or at least 99% homology thereto.

Other sequences encoding hydrogenase or related or homologous genes can be identified in a database such as, for example, GenBank. In one aspect, the gene that encodes hydrogenase is isolated from Acidithiobacillus thiooxidans and can be identified by the GI number CP045571.1 in the GenBank database. In another aspect, sequences useful herein include those with GI numbers listed in Table 6:

TABLE 6
Hydrogenase

| Source Organism | Sequence Description | GI Number |
|---|---|---|
| Acidithiobacillus thiooxidans ATCC 19377 | Genomic DNA | CP045571.1 |
| Acidithiobacillus sp. AMEEHan | Genomic DNA | CP118747.1 |
| Acidithiobacillus sp. ‘AMD consortium’ | Genomic DNA | CP044411.1 |
| Acidithiobacillus ferridurans | Genomic DNA | AP018795.1 |
| Acidithiobacillus ferrivorans | Genomic DNA | CP059488.1 |
| Acidithiobacillus ferrivorans | Genomic DNA | LT841305.1 |
| Acidithiobacillus ferrivorans SS3 | Genomic DNA | CP002985.1 |
| Acidithiobacillus ferriphilus | Genomic DNA | CP116458.1 |
| Acidithiobacillus sp. YTS05 | Genomic DNA | CP094359.1 |
| Acidithiobacillus ferrooxidans | Genomic DNA | CP084172.1 |
| Acidithiobacillus ferrooxidans | Genomic DNA | AP025160.1 |
| Acidithiobacillus ferrooxidans | Genomic DNA | CP082238.1 |
| Acidithiobacillus ferrooxidans | Genomic DNA | CP040511.1 |
| Acidithiobacillus ferrooxidans ATCC 23270 | Genomic DNA | CP001219.1 |
| Acidithiobacillus ferrooxidans ATCC 53993 | Genomic DNA | CP001132.1 |
| Sulfuriferula plumbiphila | Genomic DNA | AP021884.1 |
| Acidithiobacillus caldus | Genomic DNA | CP133598.1 |
| Acidithiobacillus caldus ATCC 51756 | Genomic DNA | CP005986.1 |
| Acidithiobacillus caldus SM-1 | Genomic DNA | CP002573.1 |
| Acidithiobacillus caldus | Genomic DNA | CP043926.1 |
| Acidithiobacillus caldus | Genomic DNA | CP026328.2 |
| Variovorax sp. PBL-H6 | Genomic DNA | LR594659.1 |
| Cupriavidus sp. KK10 | Genomic DNA | CP073678.1 |
| Sulfurimicrobium lacus | Genomic DNA | AP022853.1 |
| Cupriavidus necator N-1 | Genomic DNA | CP002879.1 |
| Cupriavidus sp. WKF15 | Genomic DNA | CP119573.1 |
| Polaromonas sp. P1-6 | Genomic DNA | CP087966.1 |
| Polaromonas sp. P1(28)-13 | Genomic DNA | CP087963.1 |
| Polaromonas sp. P2-4 | Genomic DNA | CP087965.1 |
| Variovorax paradoxus B4 | Genomic DNA | CP003912.1 |
| Gallionella capsiferriformans ES-2 | Genomic DNA | CP002159.1 |
| Polaromonas sp. P1(28)-8 | Genomic DNA | CP087964.1 |
| uncultured bacterium gwa2_scaffold_14 | CRISPR-Cas scaffold | KU516114.1 |
| Cupriavidus necator | Plasmid DNA | CP068436.1 |
| Burkholderia sp. HB1 | Genomic DNA | CP012193.1 |
| Burkholderiales bacterium GJ-E10 | Genomic DNA | AP014683.1 |
| uncultured bacterium | Genomic DNA | KX576128.1 |
| Denitratisoma oestradiolicum | Genomic DNA | LR778301.1 |
| Paraburkholderia dioscoreae | Genomic DNA | LR699554.1 |
| Cupriavidus necator H16 | Genomic DNA | CP039287.1 |
| Cupriavidus necator | Genomic DNA | CP129213.1 |
| Paraburkholderia terrae | Genomic DNA | AP025259.1 |
| Paraburkholderia xenovorans LB400 | Genomic DNA | CP008761.1 |
| Paraburkholderia terrae | Genomic DNA | AP024958.1 |
| Ralstonia pickettii DTP0602 | Genomic DNA | CP006667.1 |
| Cupriavidus necator | Genomic DNA | CP066018.1 |
| Cupriavidus necator H16 | Genomic DNA | AM260479.1 |
| Paraburkholderia xenovorans LB400 | Genomic DNA | CP000272.1 |
| Paraburkholderia aromaticivorans | Genomic DNA | CP051514.1 |
| Cupriavidus oxalaticus | Genomic DNA | CP038636.1 |
| Paucibacter sp. S2-9 | Genomic DNA | CP116346.1 |
| Paraburkholderia terricola | Genomic DNA | CP084255.1 |
| Paraburkholderia terricola | Genomic DNA | CP024941.1 |
| Dechloromonas sp. A34 | Genomic DNA | CP102486.1 |
| Polaromonas sp. JS666 | Genomic DNA | CP000316.1 |
| Paraburkholderia aromaticivorans | Genomic DNA | CP022991.1 |
| Paraburkholderia pallida | Genomic DNA | CP038152.1 |
| Variovorax sp. PBL-E5 | Plasmid DNA | LR594673.1 |
| Rhodoferax ferrireducens | Genomic DNA | CP138198.1 |
| Azoarcus sp. KH32C | Genomic DNA | AP012304.1 |
| Sideroxyarcus emersonii | Genomic DNA | AP023423.1 |
| Sulfuritalea hydrogenivorans sk43H | Genomic DNA | AP012547.1 |
| Paraburkholderia xenovorans LB400 | Genomic DNA | CP008762.1 |
| Paraburkholderia xenovorans LB400 | Genomic DNA | CP000271.1 |
| Rhodoferax ferrireducens T118 | Genomic DNA | CP000267.1 |
| Ralstonia syzygii R24 | Genomic DNA | CP115944.1 |
| Ralstonia syzygii R24 | Genomic DNA | FR854086.1 |
| Sideroxydans lithotrophicus ES-1 | Genomic DNA | CP001965.1 |
| Cupriavidus metallidurans | Plasmid DNA | CP046333.1 |
| Paraburkholderia sp. PGU19 | Genomic DNA | AP023182.1 |
| Cupriavidus metallidurans CH34 | Genomic DNA | CP000353.2 |
| Candidatus Desulfobacillus denitrificans | Genomic DNA | AP021857.1 |
| Paraburkholderia dokdonensis | Genomic DNA | CP029641.1 |
| Burkholderiaceae bacterium | Genomic DNA | CP126124.1 |
| Rugosibacter aromaticivorans | Genomic DNA | CP010554.1 |
| Paraburkholderia hospita | Genomic DNA | CP024939.1 |
| Acidiferrobacter sp. SPIII_3 | Genomic DNA | CP027663.1 |
| Paraburkholderia hospita | Genomic DNA | CP026107.1 |
| Cupriavidus metallidurans | Genomic DNA | CP090526.1 |
| Cupriavidus metallidurans | Genomic DNA | CP083718.1 |
| Candidatus Accumulibacter similis | Genomic DNA | CP054595.1 |
| Aromatoleum petrolei | Genomic DNA | CP059560.1 |
| Rhodanobacteraceae bacterium | Genomic DNA | CP126120.1 |
| Paraburkholderia caribensis MBA4 | Genomic DNA | CP012747.1 |
| Sideroxydans sp. CL21 | Genomic DNA | LR699166.1 |
| Azoarcus sp. DN11 | Genomic DNA | CP021731.1 |
| Paraburkholderia terrae | Genomic DNA | AP025260.1 |
| Burkholderia thailandensis | Genomic DNA | CP023498.1 |
| Candidatus Symbiobacter mobilis CR | Genomic DNA | CP004885.1 |
| Burkholderia thailandensis MSMB121 | Genomic DNA | CP004096.1 |
| Paraburkholderia caribensis | Genomic DNA | CP065405.1 |
| Paraburkholderia phymatum STM815 | Genomic DNA | CP001045.1 |
| Methylococcus geothermalis | Genomic DNA | CP046565.1 |
| Variovorax sp. HW608 | Genomic DNA | LT607803.1 |
| Burkholderiales bacterium | Genomic DNA | CP070653.1 |
| Sulfuricella denitrificans skB26 | Genomic DNA | AP013066.1 |
| Burkholderia humptydooensis | Genomic DNA | CP013382.1 |
| Burkholderia sp. 2002721687 | Genomic DNA | CP009548.1 |
| Burkholderia humptydooensis | Genomic DNA | CP065687.1 |
| Thiomonas arsenitoxydans | Genomic DNA | FP475956.1 |

In one aspect, the DNA constructs disclosed herein include a gene that expresses a P-type ATPase. P-type ATPases are typically found in bacteria, archaea, and eukaryotes, and function as ion pumps and/or lipid pumps. P-type ATPases are also known as E1-E2 ATPases due to their interconversion between two forms (i.e., E1 and E2). P-type ATPases have a primarily α-helical structure and harness energy from ATP hydrolysis to transport a ligand across a cell membrane. Numerous P-type ATPases are recognized, typically classified into families based on affinity for particular ions (i.e., potassium, heavy metals, calcium, sodium/potassium, proton/potassium, sodium, proton, magnesium, and/or phospholipids).

In one aspect, the gene that encodes P-type ATPase is isolated from a bacterium such as, for example, Escherichia coli (e.g. Escherichia coli str. K-12 substr. MG1655 or another E. coli strain). In a further aspect, the gene that encodes P-type ATPase has SEQ ID NO. 7 or at least 70% homology thereto, at least 75% homology thereto, at least 80% homology thereto, at least 85% homology thereto, at least 90% homology thereto, at least 95% homology thereto, or at least 99% homology thereto.

Other sequences encoding P-type ATPase or related or homologous genes can be identified in a database such as, for example, GenBank. In one aspect, the gene that encodes p-type ATPase is isolated from Escherichia coli and can be identified by the GI number CP132223.1 in the GenBank database. In another aspect, sequences useful herein include those with GI numbers listed in Table 7:

TABLE 7
P-Type ATPase

| Source Organism | Sequence Description | GI Number |
|---|---|---|
| Escherichia coli | Genomic DNA | CP132223.1 |
| Escherichia coli | Genomic DNA | CP137718.1 |
| Escherichia coli | Genomic DNA | CP137709.1 |
| Escherichia coli | Genomic DNA | OY754443.1 |
| Escherichia coli | Genomic DNA | OY754353.1 |
| Escherichia coli | Genomic DNA | OY754453.1 |
| Escherichia coli | Genomic DNA | CP107281.1 |
| Escherichia coli str. | Genomic DNA | CP044355.1 |
| K-12 substr. MG1655 | | |
| Escherichia coli | Genomic DNA | CP136397.1 |
| Escherichia coli | Genomic DNA | CP136396.1 |
| Escherichia coli | Genomic DNA | CP136398.1 |
| Escherichia coli | Genomic DNA | CP135502.1 |
| Escherichia coli | Genomic DNA | CP135077.1 |
| Escherichia coli | Genomic DNA | CP134923.1 |
| Escherichia coli | Genomic DNA | CP134918.1 |
| Escherichia coli | Genomic DNA | CP134910.1 |
| Escherichia coli | Genomic DNA | CP134370.1 |
| Escherichia coli | Genomic DNA | CP125679.1 |
| Escherichia coli | Genomic DNA | CP125676.1 |
| Escherichia coli | Genomic DNA | CP125687.1 |
| Escherichia coli | Genomic DNA | CP125681.1 |
| Escherichia coli | Genomic DNA | CP125699.1 |
| Escherichia coli | Genomic DNA | CP125688.1 |
| Escherichia coli | Genomic DNA | CP125691.1 |
| Escherichia coli | Genomic DNA | CP125695.1 |
| Escherichia coli | Genomic DNA | CP125701.1 |
| Escherichia coli | Genomic DNA | CP125702.1 |
| Escherichia coli | Genomic DNA | CP125710.1 |
| Escherichia coli | Genomic DNA | CP125713.1 |
| Escherichia coli | Genomic DNA | CP125715.1 |
| Escherichia coli | Genomic DNA | CP133854.1 |
| Escherichia coli | Genomic DNA | CP133453.1 |
| Escherichia coli | Genomic DNA | CP115361.1 |
| Escherichia coli | Genomic DNA | CP115333.1 |
| Escherichia coli | Genomic DNA | CP132594.1 |
| Escherichia coli | Genomic DNA | CP132550.1 |
| Escherichia coli | Genomic DNA | CP077969.2 |
| Escherichia coli | Genomic DNA | CP132288.1 |
| Escherichia coli | Genomic DNA | CP130710.1 |
| Escherichia coli | Genomic DNA | CP130706.1 |
| Escherichia coli | Genomic DNA | CP130447.1 |
| Escherichia coli | Genomic DNA | CP082100.1 |
| Escherichia coli | Genomic DNA | CP128790.1 |
| Escherichia coli | Genomic DNA | CP128609.1 |
| Escherichia coli | Genomic DNA | CP107005.1 |
| Escherichia coli | Genomic DNA | CP127119.1 |
| Escherichia coli | Genomic DNA | CP122496.1 |
| Escherichia coli | Genomic DNA | CP123255.1 |
| Escherichia coli | Genomic DNA | CP122317.1 |
| Escherichia coli | Genomic DNA | CP122315.1 |
| Escherichia coli | Genomic DNA | CP122316.1 |
| Escherichia coli | Genomic DNA | CP122318.1 |
| Escherichia coli | Genomic DNA | CP122319.1 |
| Escherichia coli | Genomic DNA | CP125731.1 |
| Escherichia coli | Genomic DNA | CP125621.1 |
| Escherichia coli | Genomic DNA | CP110018.1 |
| Escherichia coli | Genomic DNA | CP110017.1 |
| Escherichia coli | Genomic DNA | CP110016.1 |
| Escherichia coli | Genomic DNA | CP110015.1 |
| Escherichia coli | Genomic DNA | CP110014.1 |
| Escherichia coli | Genomic DNA | CP125059.1 |
| Escherichia coli | Genomic DNA | CP125009.1 |
| Escherichia coli | Genomic DNA | CP125039.1 |
| Escherichia coli | Genomic DNA | CP125045.1 |
| Escherichia coli | Genomic DNA | CP124995.1 |
| Escherichia coli | Genomic DNA | CP125003.1 |
| Escherichia coli | Genomic DNA | CP124986.1 |
| Escherichia coli | Genomic DNA | CP124994.1 |
| Escherichia coli | Genomic DNA | CP124970.1 |
| Escherichia coli | Genomic DNA | CP124979.1 |
| Escherichia coli | Genomic DNA | CP122625.1 |
| Escherichia coli | Genomic DNA | CP122832.1 |
| Escherichia coli | Genomic DNA | CP122872.1 |
| Escherichia coli | Genomic DNA | CP122876.1 |
| Escherichia coli | Genomic DNA | CP122895.1 |
| Escherichia coli | Genomic DNA | CP122923.1 |
| Escherichia coli | Genomic DNA | CP122929.1 |
| Escherichia coli | Genomic DNA | CP110117.1 |
| Escherichia coli | Genomic DNA | CP123237.1 |
| Escherichia coli | Genomic DNA | AP027953.1 |
| Escherichia coli | Genomic DNA | CP099032.1 |
| Escherichia coli | Genomic DNA | CP099034.1 |
| Escherichia coli | Genomic DNA | CP099062.1 |
| Escherichia coli | Genomic DNA | CP099065.1 |
| Escherichia coli | Genomic DNA | CP099087.1 |
| Escherichia coli | Genomic DNA | CP099092.1 |
| Escherichia coli | Genomic DNA | CP099095.1 |
| Escherichia coli | Genomic DNA | CP099106.1 |
| Escherichia coli | Genomic DNA | CP099118.1 |
| Escherichia coli | Genomic DNA | CP099179.1 |
| Escherichia coli | Genomic DNA | CP099294.1 |
| Escherichia coli | Genomic DNA | CP099027.1 |
| Escherichia coli | Genomic DNA | CP099059.1 |
| Escherichia coli | Genomic DNA | CP099068.1 |
| Escherichia coli | Genomic DNA | CP099072.1 |
| Escherichia coli | Genomic DNA | CP099075.1 |
| Escherichia coli | Genomic DNA | CP099089.1 |
| Escherichia coli | Genomic DNA | CP099166.1 |
| Escherichia coli | Genomic DNA | CP099178.1 |
| Escherichia coli | Genomic DNA | CP049112.1 |

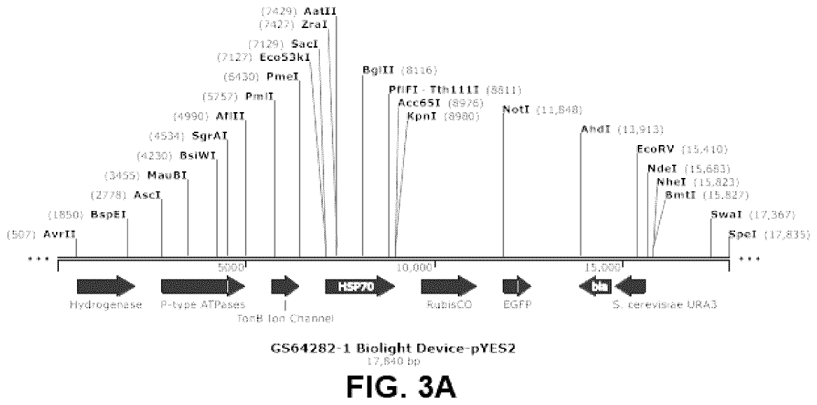

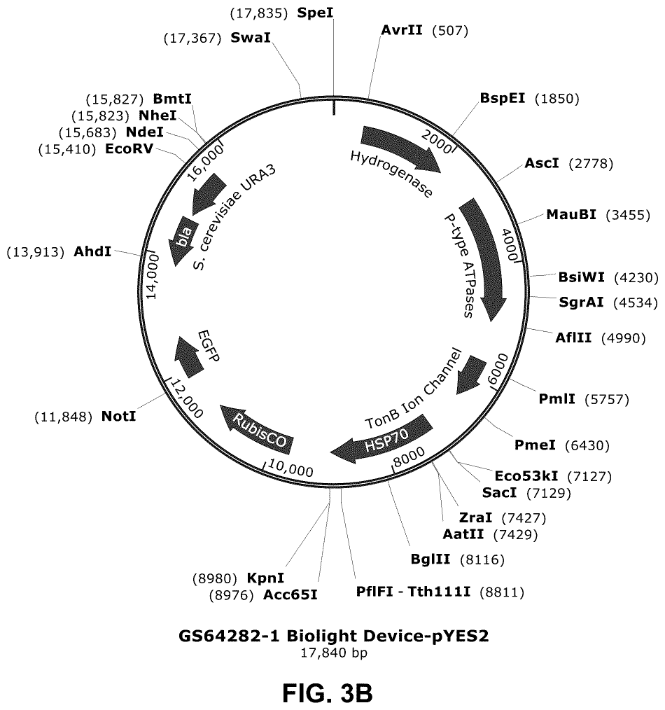

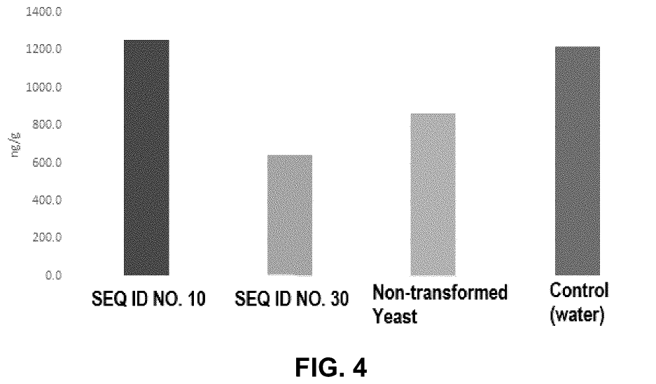

In one aspect, the DNA construct has the following genetic components: a) a gene that encodes HSP70, b) a gene that encodes RuBisCO large subunit 1, c) a gene that encodes tonB, d) a gene that encodes phosphoenol pyruvate carboxykinase, e) a gene that encodes phosphoenol pyruvate carboxylase, f) a gene that encodes a hydrogenase, and f) a gene that encodes a p-type ATPase. In some aspects, the DNA construct lacks the gene that encodes phosphoenol pyruvate carboxykinase and/or the gene that encodes phosphoenol pyruvate carboxylase. An exemplary device lacking these two genes is shown in FIGS. 3A-3B and has SEQ ID NO. 30.

In another aspect, said construct further includes a) a promoter, b) a terminator or stop sequence, c) a gene that confers resistance to an antibiotic (a “selective marker”), d) a reporter protein, or any combination thereof. Each of these elements is described in further detail below.

In one aspect, the construct includes from 5′ to 3′ the following genetic components in the following order: (1) a gene that encodes HSP70, (2) a gene that encodes RuBisCO large subunit 1, (3) a gene that encodes tonB, (4) a gene that encodes phosphoenol pyruvate carboxykinase, (5) a gene that encodes phosphoenol pyruvate carboxylase, (6) a gene that encodes hydrogenase, and (7) a gene that encodes a p-type ATPase.

In one aspect, the construct includes from 5′ to 3′ the following genetic components in the following order: a gene that encodes HSP70 having SEQ ID NO. 1 or at least 70% homology thereto, a gene that encodes RuBisCO large subunit 1 having SEQ ID NO. 1 or at least 70% homology thereto, a gene that encodes tonB having SEQ ID NO. 3 or at least 70% homology thereto, a gene that encodes phosphoenol pyruvate carboxylase having SEQ ID NO. 4 or at least 70% homology thereto, a gene that encodes phosphoenol pyruvate carboxylase having SEQ ID NO. 5 or at least 70% homology thereto, a gene that encodes a hydrogenase having SEQ ID NO. 6 or at least 70% homology thereto; and a gene that encodes a p-type ATPase having SEQ ID NO. 7 or at least 70% homology thereto.

In another aspect, the construct includes from 5′ to 3′ the following genetic components in the following order: (1) a gene that encodes HSP70; (2) a gene that encodes RuBisCO large subunit 1; (3) a gene that encodes tonB; (4) an rrnB terminator; (5) an araBAD promoter; (6) a gene that encodes phosphoenol pyruvate carboxykinase; (7) a gene that encodes phosphoenol pyruvate carboxylase; (8) an rrnB terminator; (9) an araBAD promoter; (10) a gene that encodes hydrogenase; and (11) a gene that encodes p-type ATPase.

In another aspect, the construct includes from 5′ to 3′ the following genetic components in the following order: (1) a gene that encodes HSP70 having SEQ ID NO. 1 or at least 90% homology thereto; (2) a gene that encodes RuBisCO large subunit 1 having SEQ ID NO. 2 or at least 90% homology thereto; (3) a gene that encodes tonB; having SEQ ID NO. 3 or at least 90% homology thereto (4) an rrnB terminator; (5) an araBAD promoter; (6) a gene that encodes phosphoenol pyruvate carboxykinase having SEQ ID NO. 4 or at least 90% homology thereto; (7) a gene that encodes phosphoenol pyruvate carboxylase having SEQ ID NO. 5 or at least 90% homology thereto; (8) an rrnB terminator; (9) an araBAD promoter; (10) a gene that encodes hydrogenase having SEQ ID NO. 6 or at least 90% homology thereto; and (11) a gene that encodes p-type ATPase having SEQ ID NO. 7 or at least 90% homology thereto. In some aspects the construct further includes (12) a gene that encodes a reporter protein having SEQ ID NO. 8 or at least 90% homology thereto, wherein the gene that encodes the reporter protein is positioned after the gene that encodes p-type ATPase in a 5′ to 3′ direction.

In still another aspect, the construct is a pBAD plasmid having from 5′ to 3′ the following genetic components in the following order: (1) a gene that encodes HSP70 having SEQ ID NO. 1 or at least 70% homology thereto; (2) a gene that encodes RuBisCO large subunit 1 having SEQ ID NO. 2 or at least 70% homology thereto; (3) a gene that encodes tonB; having SEQ ID NO. 3 or at least 70% homology thereto (4) an rrnB terminator; (5) an araBAD promoter; (6) a gene that encodes phosphoenol pyruvate carboxykinase having SEQ ID NO. 4 or at least 70% homology thereto; (7) a gene that encodes phosphoenol pyruvate carboxylase having SEQ ID NO. 5 or at least 70% homology thereto; (8) an rrnB terminator; (9) an araBAD promoter; (10) a gene that encodes hydrogenase having SEQ ID NO. 6 or at least 70% homology thereto; and (11) a gene that encodes p-type ATPase having SEQ ID NO. 7 or at least 70% homology thereto. In some aspects the construct further includes (12) a gene that encodes a reporter protein having SEQ ID NO. 8 or at least 70% homology thereto, wherein the gene that encodes the reporter protein is positioned after the gene that encodes p-type ATPase in a 5′ to 3′ direction.

In another aspect, the DNA construct has SEQ ID NO. 9 or at least 70% homology thereto, at least 75% homology thereto, at least 80% homology thereto, at least 85% homology thereto, at least 90% homology thereto, at least 95% homology thereto, or at least 99% homology thereto.

In one aspect, the construct includes from 5′ to 3′ the following genetic components in the following order: (1) a gene that encodes hydrogenase, (2) a gene that encodes p-type ATPase, (3) a gene that encodes phosphoenol pyruvate carboxylase, (4) a gene that encodes phosphoenol pyruvate carboxykinase, (5) a gene that encodes tonB, (6) a gene that encodes HSP70, and (7) a gene that encodes RuBisCO large subunit 1.

In one aspect, the construct includes from 5′ to 3′ the following genetic components in the following order: (1) a gene that encodes hydrogenase having SEQ ID NO. 6 or at least 70% homology thereto, (2) a gene that encodes p-type ATPase having SEQ ID NO. 7 or at least 70% homology thereto, (3) a gene that encodes phosphoenol pyruvate carboxylase having SEQ ID NO. 5 or at least 70% homology thereto, (4) a gene that encodes phosphoenol pyruvate carboxykinase having SEQ ID NO. 4 or at least 70% homology thereto, (5) a gene that encodes ton B having SEQ ID NO. 3 or at least 70% homology thereto, (6) a gene that encodes HSP70 having SEQ ID NO. 1 or at least 70% homology thereto, and (7) a gene that encodes RuBisCO large subunit 1 having SEQ ID NO. 2 or at least 70% homology thereto.

In another aspect, the construct includes from 5′ to 3′ the following genetic components in the following order: (1) a gene that encodes hydrogenase; (2) a CYC1 terminator; (3) a GAL1 promoter; (4) a gene that encodes p-type ATPase; (5) a CYC1 terminator; (6) a GAL1 promoter; (7) a gene that encodes phosphoenol pyruvate carboxylase; (8) a CYC1 terminator; (9) a GAL1 promoter; (10) a gene that encodes phosphoenol pyruvate carboxykinase; (11) a CYC1 terminator; (12) a GAL1 promoter; (13) a gene that encodes tonB; (14) a CYC1 terminator; (15) a GAL1 promoter; (16) a gene that encodes HSP70; (17) a CYC1 terminator; (18) a GAL1 promoter; and (19) a gene that encodes RuBisCO large subunit 1.

In another aspect, the construct includes from 5′ to 3′ the following genetic components in the following order: (1) a gene that encodes hydrogenase having SEQ ID NO. 6 or at least 90% homology thereto; (2) a CYC1 terminator; (3) a GAL1 promoter; (4) a gene that encodes p-type ATPase having SEQ ID NO. 7 or at least 90% homology thereto; (5) a CYC1 terminator; (6) a GAL1 promoter; (7) a gene that encodes phosphoenol pyruvate carboxylase having SEQ ID NO. 5 or at least 90% homology thereto; (8) a CYC1 terminator; (9) a GAL1 promoter; (10) a gene that encodes phosphoenol pyruvate carboxykinase having SEQ ID NO. 4 or at least 90% homology thereto; (11) a CYC1 terminator; (12) a GAL1 promoter; (13) a gene that encodes tonB having SEQ ID NO. 3 or at least 90% homology thereto; (14) a CYC1 terminator; (15) a GAL1 promoter; (16) a gene that encodes HSP70 having SEQ ID NO. 1 or at least 90% homology thereto; (17) a CYC1 terminator; (18) a GAL1 promoter; and (19) a gene that encodes RuBisCO large subunit 1 having SEQ ID NO. 2 or at least 90% homology thereto.

In still another aspect, the construct is a pYES2 plasmid having from 5′ to 3′ the following genetic components in the following order: (1) a gene that encodes hydrogenase having SEQ ID NO. 6 or at least 90% homology thereto; (2) a gene that encodes p-type ATPase having SEQ ID NO. 7 or at least 90% homology thereto; (3) a gene that encodes phosphoenol pyruvate carboxylase having SEQ ID NO. 5 or at least 90% homology thereto; (4) a gene that encodes phosphoenol pyruvate carboxykinase having SEQ ID NO. 4 or at least 90% homology thereto; (5) a gene that encodes tonB having SEQ ID NO. 3 or at least 90% homology thereto; (6) a gene that encodes HSP70 having SEQ ID NO. 1 or at least 90% homology thereto; and (7) a gene that encodes RuBisCO large subunit 1 having SEQ ID NO. 2 or at least 90% homology thereto. In some aspects the construct further includes (8) a gene that encodes a reporter protein having SEQ ID NO. 8 or at least 90% homology thereto, wherein the gene that encodes the reporter protein is positioned after the gene that encodes RuBisCO large subunit 1 in a 5′ to 3′ direction.

In still another aspect, the construct is a pYES2 plasmid having from 5′ to 3′ the following genetic components in the following order: (1) a gene that encodes hydrogenase having SEQ ID NO. 6 or at least 70% homology thereto; (2) a CYC1 terminator; (3) a GAL1 promoter; (4) a gene that encodes p-type ATPase having SEQ ID NO. 7 or at least 70% homology thereto; (5) a CYC1 terminator; (6) a GAL1 promoter; (7) a gene that encodes phosphoenol pyruvate carboxylase having SEQ ID NO. 5 or at least 70% homology thereto; (8) a CYC1 terminator; (9) a GAL1 promoter; (10) a gene that encodes phosphoenol pyruvate carboxykinase having SEQ ID NO. 4 or at least 70% homology thereto; (11) a CYC1 terminator; (12) a GAL1 promoter; (13) a gene that encodes tonB having SEQ ID NO. 3 or at least 70% homology thereto; (14) a CYC1 terminator; (15) a GAL1 promoter; (16) a gene that encodes HSP70 having SEQ ID NO. 1 or at least 70% homology thereto; (17) a CYC1 terminator; (18) a GAL1 promoter; and (19) a gene that encodes RuBisCO large subunit 1 having SEQ ID NO. 2 or at least 70% homology thereto. In some aspects the construct further includes (12) a gene that encodes a reporter protein having SEQ ID NO. 8 or at least 70% homology thereto, wherein the gene that encodes the reporter protein is positioned after the gene that encodes RuBisCO large subunit 1 in a 5′ to 3′ direction.

In another aspect, the DNA construct has SEQ ID NO. 10 or at least 70% homology thereto, at least 75% homology thereto, at least 80% homology thereto, at least 85% homology thereto, at least 90% homology thereto, at least 95% homology thereto, or at least 99% homology thereto.

### Additional Components of the DNA Constructs

In another aspect, said construct further includes a) a promoter, b) a terminator or stop sequence, c) a gene that confers resistance to an antibiotic (a “selective marker”), d) a reporter protein, or any combination thereof.

In one aspect, the construct includes a regulatory sequence. In a further aspect, the regulatory sequence is already incorporated into a vector such as, for example, a plasmid, prior to genetic manipulation of the vector. In another aspect, the regulatory sequence can be incorporated into the vector through the use of restriction enzymes or any other technique known in the art.

In one aspect, the regulatory sequence is a promoter. The term “promoter” refers to a DNA sequence capable of controlling the expression of a coding sequence. In another aspect, the coding sequence to be controlled is located 3′ to the promoter. In still another aspect, the promoter is derived from a native gene. In an alternative aspect, the promoter is composed of multiple elements derived from different genes and/or promoters. A promoter can be assembled from elements found in nature, from artificial and/or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, such as, for example, in response to the presence or absence of a particular metabolite (e.g., lactose or tryptophan), a metal ion, a molecule secreted by a pathogen, or the like. A promoter also may be regulated physically, such as, for example, in response to heat, cold, water stress, salt stress, oxygen concentration, illumination, wounding, or the like.

Promoters that are useful to drive expression of the nucleotide sequences described herein are numerous and familiar to those skilled in the art. Suitable promoters include, but are not limited to, the following: T3 promoter, T7 promoter, an iron promoter, araBAD promoter, and GAL1 promoter. In a further aspect, the promoter is a native part of the vector used herein. Variants of these promoters are also contemplated. The skilled artisan will be able to use site-directed mutagenesis and/or other mutagenesis techniques to modify the promoters to promote more efficient function. The promoter may be positioned, for example, from 10-100 nucleotides from a ribosomal binding site.

In one aspect, the promoter is a GAL1 promoter. In another aspect, the GAL1 promoter is native to the plasmid used to create the vector. In another aspect, a GAL1 promoter is positioned before the gene that encodes hydrogenase, the gene that encodes p-type ATPase, the gene that encodes phosphoenol pyruvate carboxylase, the gene that encodes phosphoenol pyruvate carboxykinase, the gene that encodes tonB, the gene that encodes HSP70, the gene that encodes RuBisCO large subunit 1, or any combination thereof.

In another aspect, the promoter is an araBAD promoter. In a further aspect, the araBAD promoter is native to the plasmid used to create the vector. In still another aspect, an araBAD promoter is positioned before the gene that encodes the gene that encodes HSP70, the gene that encodes RuBisCO large subunit 1, the gene that encodes tonB, the gene that encodes phosphoenol pyruvate carboxykinase, the gene that encodes phosphoenol pyruvate carboxylase, the gene that encodes hydrogenase, the gene that encodes p-type ATPase, or any combination thereof.

In one aspect, the regulatory sequence is an operon such as, for example, the LAC operon or LAC operator. As used herein, an “operon” is a segment of DNA containing a group of genes wherein the group is controlled by a single promoter. Genes included in an operon are all transcribed together. In a further aspect, the operon is a LAC operon and can be induced when lactose crosses the cell membrane of the biological device.

In another aspect, the regulatory sequence is a terminator or stop sequence. As used herein, a terminator is a sequence of DNA that marks the end of a gene or operon to be transcribed. In a further aspect, the terminator is an intrinsic terminator or a Rho-dependent transcription terminator. As used herein, an intrinsic terminator is a sequence wherein a hairpin structure can form in the nascent transcript that disrupts the mRNA/DNA/RNA polymerase complex. As used herein, a Rho-dependent transcription terminator requires a Rho factor protein complex to disrupt the mRNA/DNA/RNA polymerase complex. In one aspect, the terminator is an rrnB terminator obtained from or native to the pBAD plasmid. In an alternative aspect, the terminator is a CYC1 terminator obtained from or native to the pYES2 plasmid.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2000 nucleotides in the 5′ direction from the start codon of the DNA to be transcribed, or may be located 3′ to the DNA to be transcribed. Enhancers may be “cis-acting,” that is, located on the same molecule of DNA as the gene whose expression they affect.

In one aspect, and without wishing to be bound by theory, when the plasmid is a pBAD plasmid, genes from the plasmid are ideally suited for expression in bacteria under the control of the araBAD promoter and rrnB terminator. In a further aspect, since the expression of multiple genes is regulated under the same type of promoter, all genes arranged sequentially in a group having a 5′ araBAD promoter and/or ending with an rrnB terminator can be expressed together.

### Further Components of the DNA Constructs and Methods for Making Thereof

In another aspect, the vector contains one or more ribosomal binding sites. As used herein, a “ribosomal binding site” or “rbs” is a sequence of nucleotides located 5′ to the start codon of an mRNA that recruits a ribosome to initiate protein translation. In one aspect, the ribosomal binding site can be positioned before one or more or all genes in the DNA construct, or a before a subset of genes in a DNA construct.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for restriction enzymes. Further, restriction sites may be added, disabled, or removed as required, using techniques known in the art. In one aspect, the plasmid contains restriction sites for any known restriction enzyme such as, for example, HindIII, KpnI, SacI, BamHI, BstXI, EcoRI, BasBI, NotI, XhoI, XphI, XbaI, ApaI, SalI, ClaI, EcoRV, PstI, SmaI, XmaI, SpeI, EagI, SaclI, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA at a distance of from 20 bases to over 1000 bases away from the restriction site. A variety of restriction enzymes are commercially available and their recognition sequences, as well as instructions for use (e.g., amount of DNA needed, precise volumes of reagents, purification techniques, as well as information about salt concentration, pH, optimum temperature, incubation time, and the like) are provided by enzyme manufacturers.

In one aspect, a plasmid with a polylinker containing one or more restriction sites can be digested with one restriction enzyme and a nucleotide sequence of interest can be ligated into the plasmid using a commercially-available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, usually starting just after a promoter, the order and direction of elements inserted into a plasmid can be especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleotide fragments into the plasmid.

In one aspect, different genes can be ligated into a plasmid in one pot. In this aspect, the genes will first be digested with restriction enzymes. In certain aspects, the digestion of genes with restriction enzymes provides multiple pairs of matching 5′ and 3′ overhangs that will spontaneously assemble the genes in the desired order. In another aspect, the genes and components to be incorporated into a plasmid can be assembled into a single insert sequence prior insertion into the plasmid. In a further aspect, a DNA ligase enzyme can be used to assist in the ligation process.

In another aspect, the ligation mix may be incubated in an electromagnetic chamber. In one aspect, the incubation lasts for about 1 minute, about 2 minutes, about 5 minutes, about 10 minutes, about 15 minutes, about 20 minutes, about 30 minutes, or about 1 hour.

The DNA construct described herein can be part of a vector. In general, plasmid vectors containing replicon and control sequences that are derived from species compatible with the host cell are used in connection with the hosts. The vector ordinarily carries a replication site as well as marking sequences that are capable of performing phenotypic selection in transformed cells. Plasmid vectors are well known and commercially available. Such vectors include, but are not limited to, pWLneo, pSV2cat, pOG44, pXT1, pSG, pSVK3, pBSK, pYES, pYES2, pBSKII, pET, pUC, pUC19, pBAD, and pETDuet-1 vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copier per host cell and will contain selectable markers (such as genes for antibiotic resistance) to show the skilled artisan to select host eels that have been successfully transfected with the plasmids (for example, by growing the host cells in a medium containing the antibiotic).

In one aspect, the vector encodes a selection marker. In a further aspect, the selection marker is a gene that confers resistance to an antibiotic. In certain aspects, during fermentation of host cells transformed with the vector, the cells are contacted with the antibiotic. For example, the antibiotic may be included in the culture medium. Cells that have not been successfully transformed cannot survive in the presence of the antibiotic; only cells containing the vector, which confers antibiotic resistance, can survive. Optimally, only cells containing the vector to be expressed will be cultured, as this will result in the highest production efficiency of the desired gene products (e.g., peptides). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isonizid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

In certain aspects, the DNA construct can include a gene that encodes a reporter protein. The selection of the reporter protein can vary. For example, the reporter protein can be a yellow fluorescent protein, a red fluorescent protein, a green fluorescent protein, or a cyan fluorescent protein. In one aspect, the reporter protein is an enhanced green fluorescent protein and the gene that encodes the reporter protein has SEQ ID NO. 8 or at least 70% homology thereto. The amount of fluorescence that is produced can be correlated to the amount of DNA incorporated into the transfected cells. The fluorescence produced can be detected and quantified using techniques known in the art. For example, spectrofluorometers are typically used to measure fluorescence.

The DNA construct described herein can be part of a vector. In one aspect, the vector is a plasmid, a phagemid, a cosmid, a yeast artificial chromosome, a bacterial artificial chromosome, a virus, a phage, or a transposon.

Exemplary methods for producing the DNA constructs described herein are provided in the Examples. Restriction enzymes and purification techniques known in the art can be used to assemble the DNA constructs. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the methods described below.

### Cells and Biological Devices

A variety of different types of cells can be used in the methods described herein. In one aspect, the cells can be wild-type cells (i.e., not genetically-modified). In one aspect, the cells are from an animal such as, for example, a mammal, bird, fish, reptile, amphibian, or invertebrate. In another aspect, the cells are from a plant such as, for example, an agricultural crop, a decorative plant, a woody plant, a medicinal plant, or a combination thereof. In another aspect, the cells are from a multicellular fungus such as, for example, a mushroom, a mycorrhizal fungus, or a commercially-important mold.

In another aspect, the cells include a biological device. A “biological device” is formed when a microbial cell is transfected with a DNA construct. The biological devices are generally composed of microbial host cells, where the host cells are transformed (i.e., genetically-modified) with a DNA construct.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce an extract or composition that can remove metals and/or other contaminants from water or petroleum, such as, for example, those disclosed herein. “Heterologous” genes and proteins are genes and proteins that have been experimentally inserted into a cell that are not normally expressed by the cell. A heterologous gene may be cloned or derived from a different cell type or species than the recipient cell or organism. Heterologous genes may be introduced into cells by transduction or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes).

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the type of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed only for a limited period of time).

“Competent cells” refers to microbial cells capable of taking up heterologous DNA. Competent cells can be purchased from a commercial source, or cells can be made competent using procedures known in the art. Exemplary procedures for producing competent cells are provided in the Examples.

The host cells as referred to herein include their progeny, which are any and all subsequent generations formed by cell division. It is understood that not all progeny may be identical due to deliberate or inadvertent mutations. A host cell may be “transfected” or “transformed,” which refers to a process by which an exogenous nucleic acid is transferred or introduced into the host cell.

A transformed cell includes the primary subject cell and its progeny. The host cells can be naturally-occurring cells or “recombinant” cells. Recombinant cells are distinguishable from naturally-occurring cells in that naturally-occurring cells do not contain heterologous DNA introduced through molecular cloning procedures. In one aspect, the host cell is a prokaryotic cell such as, for example, Escherichia coli. In other aspects, the host cell is a eukaryotic cell such as, for example, the yeast Saccharomyces cerevisiae. Host cells transformed with the DNA construct described herein are referred to as “biological devices.”

The DNA construct is first delivered into the host cell. In one aspect, the host cells are naturally competent (i.e., able to take up exogenous DNA from the surrounding environment). In another aspect, cells must be treated to induce artificial competence. This delivery may be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the membranes of the cells through which the vector containing the DNA construct enters. Another method involves exposing intact yeast cells to alkali cations such as, for example, lithium. In one aspect, this method includes exposing yeast to lithium acetate, polyethylene glycol, and single-stranded DNA such as, for example, salmon sperm DNA. Without wishing to be bound by theory, the single-stranded DNA is thought to bind to the cell wall of the yeast, thereby blocking plasmids from binding. The plasmids are then free to enter the yeast cell. Enzymatic and/or electromagnetic techniques can also be used alone, or in combination with other methods, to transform microbial cells. Exemplary procedures for transforming yeast and bacteria with specific DNA constructs are provided in the Examples. In certain aspects, two or more types of DNA can be incorporated into the host cells. Thus, different metabolites can be produced from the same host cells at enhanced rates.

### Cell Culture

A satisfactory microbiological culture contains available sources of hydrogen donors and acceptors, carbon, nitrogen, sulfur, phosphorus, inorganic salts and, in certain cases, vitamins or other growth-promoting substances. For example, the addition of peptone provides a readily-available source of nitrogen and carbon. Furthermore, the use of different types of media results in different growth rates and different stationary phase densities. A rich media results in a short doubling time and higher cell density at stationary phase. Minimal media results in slow growth and low final cell densities. Efficient agitation and aeration increase final cell densities.

Culturing or fermenting of host cells can be accomplished by any technique known in the art. In one aspect, batch fermentation can be conducted. In batch fermentation, the composition of the culture medium is set at the beginning and the system is closed to future alterations. In some aspects, a limited form of batch fermentation may be carried out, wherein factors such as oxygen concentration and pH are manipulated, but additional carbon is not added. Continuous fermentation methods are also contemplated. In continuous fermentation, equal amounts of a defined medium are continuously added to and removed from a bioreactor. In other aspects, microbial cells are immobilized on a substrate. Fermentation may be carried out on any scale and may include methods in which literal “fermentation” is carried out as well as other culture methods that are non-fermentative.

In one aspect, the microorganisms can be cultured for a period of from 2 days to 2 weeks, or for about 2, about 3, about 4, about 5, about 6, about 7, about 8, about 9, about 10, about 11, about 12, about 13, or about 14 days, where any value can be the lower or upper endpoint of a range (e.g., about 3 days to about 13 days, about 8 days to about 12 days, etc.). In one aspect, the microorganisms are cultured for about 10 days.

In another aspect, the microorganisms can be cultured at any temperature appropriate for the microorganisms, with the understanding that the temperature may vary according to the microorganism (for example, a thermophilic microorganism may require a higher culture temperature than a mesophile). In one aspect, the microorganisms are cultured at a temperature of from about 20 to about 37° C., or are cultured at about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C., where any value can be the lower or upper endpoint of a range, where any value can be the lower or upper endpoint of a range (e.g., about 21° C. to about 36° C., about 25° C. to about 30° C., etc.).

In certain aspects, after culturing the microorganisms for a sufficient time, the microbial cells can be lysed with one or more enzymes. For example, when the microbial cells are fungal, the fungal cells can be lysed with lyticase. In one aspect, the lyticase concentration can be about 500 μL, about 600 μL, about 700 μL, about 800 μL, about 900 μL, or about 1,000 μL per liter of culture, where any value can be the lower or upper endpoint of a range, where any value can be the lower or upper endpoint of a range (e.g., about 500 μL to about 900 μL, about 600 μL to about 800 μL, etc.).

In addition to or in place of enzymes, other components can be used to facilitate lysis of the microbial cells. In one aspect, chitosan can be used in combination with an enzyme to lyse the microbial cells. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation. In one aspect, the chitosan is from about 60% to about 100% acetylated, or about 70%, about 75%, about 80%, about 85%, about 90%, about 95%, or about 100% acetylated, where any value can be the lower or upper endpoint of a range, where any value can be the lower or upper endpoint of a range (e.g., about 60% to about 90%, about 70% to about 80%, etc.).

The molecular weight of the chitosan can vary, as well. For example, the chitosan can comprise about 3, 4, 5, 6, 7, 8, 9, 10, 11, 12, 13, 14, 15, 16, 17, 18, 19, or 20 glucosamine units and/or N-acetylglucosamine units, where any value can be the lower or upper endpoint of a range, where any value can be the lower or upper endpoint of a range (e.g., 2 to 19, 3 to 10, 5 to 7, etc.). In one aspect, chitosan can be added until a concentration of about 0.0015%, about 0.0025%, about 0.005%, about 0.0075%, about 0.01%, about 0.015%, about 0.02%, about 0.03%, about 0.04%, or about 0.05%, where any value can be an upper or lower endpoint of a range (e.g., 0.002% to 0.04%, 0.05% to 0.015%, etc.).

In another aspect, cells can first be fermented, for example, in a biofermenter, at a temperature conducive to cell growth. In one aspect, the cells are fermented at 30° C. In a further aspect, the cells are fermented for a time period sufficient to produce the metabolite(s) of interest. In one aspect, the cells are fermented for from 6 hours to 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, during fermentation, a micro-current can be applied to the cells as described above. In some aspects, the micro-current is applied for the entire culture period. In another aspect, the micro-current is applied for only a part of the culture period, or for several non-consecutive parts of the culture period. In one aspect, the micro-current is the same throughout the entire culture period. In an alternative aspect, the micro-current is varied during the culture period.

Exemplary methods for culturing cells and/or the biological devices disclosed herein are provided in the Examples.

### Culture Medium

In some aspects, the cells are suspended in a culture medium. In another aspect, the culture medium can be Dulbecco's Modified Eagle Medium (DMEM), RPMI 1640, Minimal Essential Medium (MEM), Eagle's Minimal Essential Medium (EMEM), Iscove's Modified Dulbecco's Medium (IMDM), DMEM/F12 Medium, Murashige and Skoog (MS) medium, White's medium, Agrobacterium minimal medium, Banana AGS basal medium, Blaydes basal medium, Bold's basal medium, Chu (N6) medium, De Greef and Jacobs Medium, DKW basal medium, Economou and Read basal medium, Gamborg (B5) medium, Gresshoff and Doy medium, Heller medium, Hoagland complete medium, Jensen's medium, Kao and Michayluk medium, Litvay medium, NB basal medium, Nitsch medium. NLN medium, Quoirin and Lepoivre medium, Schenk and Hildebrandt medium, TAP medium, TM4G medium, Vacin and Went medium, wheat callus induction medium, Luria Bertani (LB) broth, terrific broth, tryptic soy broth, minimal salts (M9) medium, SOB medium, SOC medium, yeast malt medium, YPD broth, YNB broth, synthetic complete (SC) medium, YPG medium, Hartwell's complete (HC) medium, or a combination thereof. In one aspect, the culture medium is Luria Bertani (LB) broth or yeast malt medium.

In another aspect, the culture medium can contain supplemental compounds such as, for example, vitamins, nucleosides, nucleotides, amino acids, a carbohydrate, an antibiotic, or a combination thereof.

In one aspect, the culture medium can be a liquid. In another aspect, the methods disclosed herein can be performed in a biofermenter. In an alternative aspect, the cells can be distributed on a substrate. In one aspect, the substrate can be agar, a culture dish, contaminated soil, a wastewater treatment device, mineral ore, a plant organ, a tissue scaffold, or a fermentable material. When the substrate is a plant organ, in some aspects, the plant organ can be a root, leaf, stem, rhizome, tuber, flower, seed, fruit, vegetable, callus, or a combination thereof. When the substrate is a fermentable material, in some aspects, the substrate can be milk, a grain, cabbage, soybeans, fish, or a biomass feedstock. When the substrate is a biomass feedstock, in some aspects, the substrate can be forestry residue, logging residue, sawmill residue, animal manure, a recycled material, a carbohydrate waste, corn cob, corn stover, wheat straw, nut hulls, soy hulls, switchgrass, gammagrass, paper, or a combination thereof.

### Extraction and Purification of Metabolites

In one aspect, the methods disclosed herein can be used to increase the production of metabolites by cells. In some aspects, the metabolites are secreted into a culture medium and collected. In other aspects, the metabolites remain in the cells, requiring the cells to be lysed prior to collection and purification of the metabolites.

In one aspect, prior to collection of any metabolite(s) of interest, fermentation can be stopped. In some aspects, the micro-current will be withdrawn or turned off (e.g., by turning off a power supply to a biofermenter or a similar mechanism). In another aspect, an enzyme such as, for example, lyticase can optionally be used to lyse cells following fermentation. In still another aspect, the cell culture can optionally be autoclaved for a sufficient time following cell lysis in order to ensure no living cells remain in the culture. Following lysis and autoclaving, or instead of performing these two processes, centrifugation, sonication, and filtration can be performed to facilitate collection of relevant metabolites. In an alternative aspect, culture medium including an increased concentration of the desired metabolite(s) from the biofermenter can be used without further processing.

In any of the above aspects, cell cultures of biological devices such as those disclosed herein, purified metabolites, and/or extracts containing metabolites can be applied to other cells and/or tissues in order to increase metabolite production. In one aspect, the cell cultures, metabolites, and/or extracts are applied to plant tissue such as, for example, plant calluses. Following plant tissue growth, calluses can be crushed and macerated with a solvent in order to extract metabolites from the plant tissue. In one aspect, choice of solvent depends on the chemical characteristics of the metabolite being extracted. For example, lycopene would be extracted with a hydrophobic solvent.

### Applications of Genelight Extracts

In one aspect, the compositions and extracts described herein are useful in a variety of applications such as, for example, powering lighting sources, powering warming and cooling devices, and powering battery operated or other electrical devices in a variety of environments and settings.

### Lighting Applications

In one aspect, for any device or in any situation where lighting is required and where use of an LED is suitable, the compositions and extracts disclosed herein can be incorporated into a circuit, wherein the circuit can provide lighting for one or more of: cell phones, medical instruments, holiday lighting displays, handheld flashlights, indicator lights on radar or sonar equipment, digital displays of alphanumeric information, billboards, automobiles (both interior and exterior), airport runways, train tracks, subways, highways, street signs, traffic lights, auxiliary lighting for building safety, exit signs and other lighted signs, flares, night-vision equipment, telescopes, binoculars, rifle scopes, distance finders, and the like.

### Warming and Cooling Applications

In another aspect, wherever a temperature differential is required, the compositions and extracts disclosed herein can be incorporated into a circuit, wherein the circuit can provide warming and/or cooling for one or more of: clothing including, but not limited to, gloves, hats, coats, shoes, socks, blankets, or baby clothing; climate control in buildings including, but not limited to, floor heating, roof heating, building heating, water heaters, fans, and the like; food preparation and storage including but not limited to, refrigerators, freezers, beverages coolers and warmers, food warmers, ice chests, cooking apparatuses including stoves and grills, and the like. In still another aspect, the compositions and extracts disclosed herein can be used as add-ins or backups to other power sources such as, for example, gas, coal, nuclear, solar, or wind energy, or can be used during power outages instead of or in addition to generators.

### Battery-Powered and Electrical Devices

In one aspect, the compositions and methods described herein can be incorporated into a circuit, wherein the circuit can be incorporated into an electronic device such as a battery of any size including a rechargeable battery, a key fob or remote control, an electronic reading device, a laptop or portable computer, or similar. In another aspect, the circuit can be incorporated into a kitchen appliance such as, for example, a microwave, toaster, coffee maker, oven, electric knife, food and beverage slicers, grinders, mixers, and dispensers, blenders, and the like. In still another aspect, the circuit can be incorporated into a television, portable video device, radio, speaker, microphone, video game console or controller, headphones, sound canceling equipment, or similar. In still another aspect, the circuit can be incorporated into a toy, a bicycle or tricycle, a musical instrument, a prosthetic limb, pacemaker, a CPAP device, a device for stimulating muscle growth and regeneration, or an insulin pump or other powered medical implant, a thermometer, a hearing aid, eyeglasses, another medical device, a scientific instrument such as, for example, a microscope, a power tool such as an air compressor, a gas detection device, a smoke detector, or an electric toothbrush or other small personal hygiene or grooming device. In one aspect, the circuit can be used in the transportation industry, for example, in automobiles, trucks, trains, buses and other means of public transportation, subways, watercraft of various sizes, aircraft, drones, motorcycles, golf carts, and the like. In another aspect, the circuit can be used in any type of pump (i.e., water, sewage, oil field, marine, swimming pool or hot tub, artificial heart). In yet another aspect, the circuit can be incorporated into electric fencing egress and control, a clock or other timekeeping device, irrigation systems and landscaping equipment, a cash register, voting equipment, buzzers, security systems, pest control devices, and equipment for industrial or home cleaning and sterilizing.

In one aspect, the devices can be wireless devices. In an alternative aspect, the devices may optionally require wires for operation.

### Environmental Suitability

Devices and applications of the compositions and extracts disclosed herein are suitable for use in a variety of environments. In one aspect, the devices can be used in applications under water, at high altitudes, at temperature extremes both high and low, in situations where traditional batteries and/or power sources would present a flammability hazard or explosion risk, in space, in laboratories and industrial facilities, in remote locations, and in areas with weather extremes including wind, rain, sandstorms, and the like.

### Methods for Enhancing the Growth of Microorganisms

In one aspect, the compositions and extracts disclosed herein can be used as a culture medium for commercially important microorganisms, or can be used as a supplement to an existing medium. Further in this aspect, culturing microorganisms in the presence of the compositions and extracts disclosed herein can lead to enhanced growth rates and/or enhanced production of desirable metabolites.

In a further aspect, the compositions and extracts disclosed herein are useful in culturing the following types or organisms: (1) Saccharomyces cerevisiae for use in yeast doughs, brewing beer and wine, genetic research, and production of desirable secondary metabolites including the enzymes invertase and raffinase; Kluyveromyces species for the commercial production of lactase; and Candida species for the commercial production of lipase; (2) Lactobacillus species for use in making fermented foods such as yogurt, kefir, cheese, sauerkraut, pickles, hard cider, wine, and beer as well as for the commercial production of lactic acid and engineered Lactobacillus species engineered to produce protein drugs such as, for example, insulin; (3) Pyrococcus furiosus, Thermus aquaticus, Bacillus stearothermophilus, Thermus filiformis, Thermus thermophilus, and other thermophiles for production of heat stable polymerases for use in the polymerase chain reaction (PCR); (4) Xanthomonas species for production of xanthan gum, used in a variety of food and cosmetic products; (5) Aspergillus niger, used in the production of citric acid and fermentation of sake and other alcoholic beverages and this and other Aspergillus species for commercial production of α-amylase, aminoacylase, glucoamylase, catalase, glucose oxidase, lactase, pectinase, pectin lyase, and protease; Trichoderma species for the commercial production of cellulose; Mucor miehei for the commercial production of rennet; Rhizopus species for the commercial production of lipase; and Mortierella species for the commercial production of raffinase; (6) Clostridium species for production of botulinum toxin for cosmetic and medical purposes as well as the production of butanol (i.e., from Clostridium acetobutylicum); (7) Streptomyces species for production of antibiotics, antiparasitic, antineoplastic, and antifungal compounds including, but not limited to, chloramphenicol, daptomycin, fosfomycin, lincomycin, neomycin, nourseothricin, puromycin, streptomycin, tetracycline, oleandomycin, tunicamycin, mycangimycin, boromycin, bambermycin, clavulanic acid, guadinomine, ivermectin, migrastatin, bleomycin, erythromycin, geldanamycin, and the like; (8) Penicillium species for the production of penicillin and other beta-lactam antibiotics and precursors to semi-synthetic beta-lactam antibiotics; (9) Acetobacter aceti for production of acetic acid; (10) Bacillus species for commercial production of α-amylase, β-amylase, glucose isomerase, penicillin amidase, and protease; E. coli for commercial production of asparaginase; and Klebsiella species for commercial production of pullulanase; (11) Trichoderma polysporum for the production of cyclosporine A, (12) yeasts such as Monascus purpureus for the production of statin drugs for lowering blood cholesterol; (13) Bacillus thuringiensis for the production of insecticides, and other commercially important bacteria, fungi, algae, and cyanobateria.

In an alternative aspect, the compositions and extracts disclosed herein can be used as an animal food or animal supplement. Further in this aspect, the compositions and extracts disclosed herein may contain vitamins, minerals, proteins, peptides, carbohydrates, and/or other nutrients essential for animal growth and development and/or maintenance of animal health.

### Methods for Enhancing the Physiological Properties of Plants

The compositions and extracts described herein can enhance or improve the physiological properties of a plant. The term “physiological property” as defined herein includes any physical, chemical, or biological feature that is improved using the compositions and extracts described herein. In one aspect, the compositions and extracts can enhance the growth rate of the plant.

In another aspect, the physiological property includes increased root tension, root length, hormone production, drought tolerance, disease resistance, photosynthesis, number of fruits harvested for a fruiting plant, average weight of fruits harvested, phytochrome and/or chlorophyll production, or any combination thereof.

Herein, “plant” is used in a broad sense to include, for example, any species of woody, ornamental, crop, cereal, fruit, or vegetable plant, as well as photosynthetic green algae. “Plant” also refers to a plurality of plant cells that are differentiated into a structure that is present at any stage of the plant's development. Such structures include, but are not limited to, fruits, shoots, stems, leaves, flower petals, roots, tubers, corms, bulbs, seeds, gametes, cotyledons, hypocotyls, radicles, embryos, gametophytes, tumors, and the like. “Plant cell,” “plant cells,” or “plant tissue” as used herein refer to differentiated and undifferentiated tissues of plants including those present in any of the tissues described above, as well as to cells in culture such as, for example, single cells, protoplasts, embryos, calluses, etc.

The selection of the plant used in the methods described herein can vary depending on the application. For example, a specific plant can be selected that produces certain desirable metabolites. Current techniques for producing most plant metabolites are expensive. For example, large amounts of fresh plant biomass must be cultivated and harvested and expensive and time-consuming extraction methods must be used. The compositions and extracts described herein enhance the production of metabolites from plants that naturally produce these metabolites.

In one aspect, plant cells when contacted with the compositions and extracts described herein exhibit enhanced production of various desirable metabolites. Recipient cell targets include, but are not limited to, meristem cells, Type I, Type II, and Type Ill callus, immature embryos and gametic cells such as microspores, pollen, sperm, and egg cells. It is contemplated that any cell from which a fertile plant may be regenerated is useful as a recipient cell. Type I, Type II, and Type Ill callus may be initiated from tissue sources including, but not limited to, immature embryos, immature inflorescences, seedling apical meristems, microspores, and the like. Those cells that are capable of proliferating as callus are also useful herein. Methods for growing plant cells are known in the art. In one aspect, plant calluses grown from 2 to 4 weeks can be used herein. The plant cells can also be derived from plants varying in age. For example, plants that are 80 days to 120 days old after pollination can be used to produce calluses useful herein.

The plant cells can be contacted with the compositions and extracts described herein in a number of different ways. In one aspect, the compositions and extracts described herein can be added to media containing the plant cells, or can be the media containing the plant cells. In another aspect, the compositions and extracts can be injected into the plant cells via syringe. The amount of extract and the duration of exposure to the extract can vary as well.

Once the plant cells have been in contact with the compositions and extracts for a sufficient time to produce a desired metabolite, the metabolite is isolated. In one aspect, the metabolite is extracted from the media containing the plant cells. The selection of extraction solvent can vary depending on the solubility of the metabolite.

In other aspects, the compositions and extracts described herein can increase the growth rate of a plant. In particular, the compositions and extracts described herein are effective in accelerating plant development in the early stages of tissue culturing. By accelerating plant development in the early stages, it is possible to harvest more metabolites from the plant.

Additionally, traditional methods for tissue culture involve the use of synthetic growth factors such as 2,4-dichlorophenoxyacetic acid (2,4-D), which can pose environmental concerns. The compounds and extracts described herein avoid the need for such compounds.

In certain aspects, any of the compositions and extracts described above can be used in combination with a polysaccharide to enhance one or more physiological properties of the plant. In one aspect, the plant (e.g., cells, seeds, callus, mature plant) is first contacted with the compositions and extracts described herein, then subsequently contacted with the polysaccharide. In another aspect, the plant is first contacted with the polysaccharide, then subsequently contacted with the compositions or extracts described herein. In a still further aspect, the plant cells are contacted simultaneously with the polysaccharide and the compositions and extracts described herein.

In one aspect, the polysaccharide includes a polyactive carbohydrate as described and produced in WO 2019/055456, which is incorporated by reference in its entirety with respect to polyactive carbohydrates.

In one aspect, the polysaccharide includes chitosan, glucosamine (GlcN), N-acetylglucosamine (NAG), or any combination thereof. Chitosan is generally composed of GlcN and NAG units and can be chemically or enzymatically extracted from chitin, which is a component of arthropod exoskeletons and fungal and microbial cell walls. In certain aspects, the chitosan can be acetylated to a specific degree of acetylation in order to enhance tissue growth during culturing as well as metabolite production. In one aspect, chitosan isolated from shells of crab, shrimp, lobster, and/or krill is useful herein.

In one aspect, the polysaccharide is in a solution of water and acetic acid at less than 1% by weight, less than 0.75% by weight, less than 0.5% by weight, less than 0.25% by weight, or less than 0.1% by weight. In another aspect, the amount of chitosan that is applied to the plant cells is from 0.01 wt % to 0.1 wt % by weight, or 0.01 wt %, 0.02 wt %, 0.03 wt %, 0.04 wt %, 0.05 wt %, 0.06 wt %, 0.07 wt %, 0.08 wt %, 0.09 wt %, or 0.1 wt %, where any value can be an upper or lower endpoint of a range (e.g., 0.01 wt % to 0.09 wt %, 0.02 wt % to 0.08 wt %, etc.). The polysaccharides used herein are generally natural polymers and thus present no environmental concerns. Additionally, the polysaccharides can be used in acceptably low concentrations. In certain aspects, however, the polysaccharides can be used in combination with one or more plant growth regulators.

In one aspect, the polysaccharide is in a solution of water and acetic acid at less than 1% by weight, less than 0.75% by weight, less than 0.5% by weight, less than 0.25% by weight, or less than 0.1% by weight. In another aspect, the amount of chitosan that is applied to the plant cells is from 0.01 wt % to 0.1 wt % by weight, or 0.01 wt %, 0.02 wt %, 0.03 wt %, 0.04 wt %, 0.05 wt %, 0.06 wt %, 0.07 wt %, 0.08 wt %, 0.09 wt %, or 0.1 wt %, where any value can be an upper or lower endpoint of a range (e.g., 0.01 wt % to 0.09 wt %, 0.02 wt % to 0.08 wt %, etc.). The polysaccharides used herein are generally natural polymers and thus present no environmental concerns. Additionally, the polysaccharides can be used in acceptably low concentrations. In certain aspects, however, the polysaccharides can be used in combination with one or more plant growth regulators.

In one aspect, disclosed herein are compositions composed of the genelight extracts described herein with one or more polysaccharides. In one aspect, the composition includes water. In one aspect, the composition includes the genelight extract, chitosan, polyactive carbohydrate, and water. In one aspect, the genelight extract is less than 1% by weight, less than 0.75% by weight, less than 0.5% by weight, less than 0.25% by weight, less than 0.1% by weight, less than 0.5% by weight, or less than 0.3% by weight of the composition. In one aspect, the polysaccharide is chitosan and is less than 1% by weight, less than 0.75% by weight, less than 0.5% by weight, less than 0.25% by weight, less than 0.1% by weight, less than 0.5% by weight, or less than 0.3% by weight of the composition. In one aspect, the polysaccharide is a polyactive carbohydrate and is less than 1% by weight, less than 0.75% by weight, less than 0.5% by weight, less than 0.25% by weight, less than 0.1% by weight, less than 0.5% by weight, or less than 0.3% by weight of the composition.

In one aspect, the plant growth regulator is an auxin, a cytokinin, a gibberellin, abscisic acid, or a polyamine. In a further aspect, the auxin is a natural or synthetic auxin. In a still further aspect, the auxin is indole-3-acetic acid (IAA), 4-chloroindole-3-acetic acid (4-Cl-IAA), 2-phenylacetic acid (PAA), indole-3-butyric acid (IBA), 2,3-dichlorophenoxyacetic acid (2,4-D), α-naphthalene acetic acid (α-NAA), 2-methoxy-3,6-dichlorobenzoic acid (dicamba), 4-amino-3,5,6-trichloropicolinic acid (torden or picloram), 2,4,5-trichloropicolinic acid (2,4,5-T), or a combination thereof. In another aspect, the cytokinin is zeatin, kinetin, 6-benzylaminopurine, diphenylurea, thidizuron (TDZ), 6-(γ,γ-dimethylallylamino)purine, or a combination thereof. In another aspect, the gibberellin is gibberellin A1 (GA1), gibberellic acid (GA3), ent-gibberellane, ent-kaurene, or a combination thereof. In yet another aspect, the polyamine is putrescine, spermidine, or a combination thereof.

In one aspect, the plant cell or callus is first contacted with a polysaccharide and subsequently contacted with a plant growth regulator. In another aspect, the plant cell or callus is first contacted with a plant growth regulator and subsequently contacted with a polysaccharide. In an alternative aspect, the plant cell or callus is simultaneously contacted with a polysaccharide and a plant growth regulator. In a further aspect, the plant cell or callus is only contacted with a polysaccharide and is not contacted with a plant growth regulator.

The plant cells can be contacted with the polysaccharide using a number of techniques. In one aspect, the plant cells or reproductive organs (e.g., a plant embryo) can be cultured in agar and medium with a solution of the polysaccharide. In other aspects, the polysaccharide can be applied to a plant callus by techniques such as, for example, coating the callus or injecting the polysaccharide into the callus. In this aspect, the age of callus can vary depending on the type of plant. The amount of polysaccharide can vary depending upon, among other things, the selection and number of plant cells. The use of the polysaccharide in the methods described herein permits rapid tissue culturing at room temperature. Due to the ability of the polysaccharide to prevent microbial contamination, the tissue can grow for extended periods of time ranging from days to several weeks. Moreover, tissue culturing with the polysaccharide can occur in the dark and/or light. As discussed above, the plant cells are also contacted with any of the compositions or extracts described above. Thus, the use of the polysaccharides and compositions and extracts described herein is a versatile way to culture and grow plant cells—and, ultimately, plants of interest—with enhanced physiological properties.

In other aspects, the plant cells can be cultured in a liquid medium on a larger scale in a bioreactor. For example, plant cells can be cultured in agar and medium, then subsequently contacted with the compositions and extracts described herein. After a sufficient culturing time (e.g., two to four weeks), the plant cells are introduced into a container with the same medium used above and, additionally, the polysaccharide. In certain aspects, the polysaccharide can be introduced with anionic polysaccharides including, but not limited to, alginates (e.g., sodium alginate, calcium alginate, potassium alginate, etc.). After the introduction of the polysaccharide, if using, the solution is mixed for a sufficient time to produce a desired result (e.g., production of a desired metabolite). Alternatively, the initial liquid medium in the bioreactor can include any of the compositions and/or extracts described herein.

In one aspect, provided herein is a plant grown by the process that involves contacting plant gamete cells or a plant reproductive organ with the extracts disclosed herein. In a further aspect, the plant is produced by the following method:
(a) contacting a plant callus with the extracts;
(b) culturing the plant callus; and
(c) growing the plant from the plant callus.

In a further aspect, the same method can be applied to other plant parts including fruits, stems, roots, tubers, corms, bulbs, flowers, buds, seeds, and the like. In a still further aspect, the same method can be applied to an entire plant.

In one aspect, the plant callus is immersed in a solution of polysaccharide (e.g., chitosan), then inoculated with the compositions and/or extracts described herein. In another aspect, the plant callus can be from 2 days up to 20 days old prior to inoculation with the compositions and/or extracts described herein. The plant callus is then allowed to grow until it is of sufficient weight and size. In one aspect, the plant callus is allowed to grow (i.e., culture) for 1 to 10 weeks after inoculation. Following growth or culture of the callus for a sufficient period of time, desired metabolites can be collected according to methods known in the art; said methods are specific to the desired metabolites and make use of properties ranging from molecular size to charge to hydrophobicity or hydrophilicity to other properties useful for collection and purification of the metabolites.

### Turf Applications

In one aspect, the compositions and extracts disclosed herein can be used to improve the health and appearance of grass and/or turf such as, for example, grass or turf on sports playing fields, lawns, golf courses, and the like. In one aspect, application of the compositions and extracts enhances root growth.

In a further aspect, plant hormones can promote or stimulate plant growth and development. In one aspect, application of the compounds or extracts disclosed herein stimulates endogenous production of plant hormones, in turn enhancing root growth. In a further aspect, application of the compounds or extracts increases the production of cytokinins by the plants. Cytokinins are responsible for the growth of roots and anchoring of a plant in soil. Still further in this aspect, root strength is especially important on golf courses due to foot traffic as well as contact with golf clubs. In another aspect, application of the compounds or extracts increases the production of salicylic acid by the plants. Salicylic acid promotes and/or enhances photosynthesis, transpiration, and mineral uptake. In one aspect, increased production of salicylic acid maintains an aesthetically-pleasing green appearance in, for example, turf grasses, as well as protecting the plant against pathogenic microorganisms. In still another aspect, application of the compounds or extracts increases the production of jasmonic acid by plants. Jasmonic acid protects against environmental damage, microbial pathogens, and insects, as well as protecting against early senescence of plants. Jasmonic acid is particularly important for field grasses (e.g., on golf courses) since the grasses are kept green for most of the year.

In one aspect, application of the compounds or extracts disclosed herein increases root tension (i.e., how difficult it is to pull up a plant or section of sod to which the compounds or extracts have been applied) and/or root length. In another aspect, applications of the compounds or extracts disclosed herein leads to a greener appearance of plants such as, for example, field grasses or turf grasses. In one aspect, the compositions and extracts described herein can increase the root force of sod by about 50% to about 200%, or about 50%, about 75%, about 100%, about 125%, about 150%, about 175%, or about 200% compared to sod that has not been treated with the composition or extract, where any value can be a lower and upper endpoint of a range (e.g., about 50% to about 175%, about 75% to about 125%, etc.).

In one aspect, the compositions and extracts described herein can increase the root force of a plant by about 50% to about 200%, or about 50%, about 75%, about 100%, about 125%, about 150%, about 175%, or about 200% compared to a plant that has not been treated with the composition or extract, where any value can be a lower and upper endpoint of a range (e.g., about 50% to about 175%, about 75% to about 125%, etc.).

In one aspect, the compounds and extracts disclosed herein can be applied at a volume of about 50 to about 200 mL per square meter of, for example, turf, or can be applied at about 50 mL, about 75 mL, about 100 mL, about 125 mL, about 150 mL, about 175 mL, or about 200 mL per square meter of turf, or a combination of any of the foregoing values, or a range encompassing any of the foregoing values. In one aspect, the compounds and extracts are applied to turf at a volume of about 75 mL per square meter. In an alternative aspect, the compounds and extracts are applied to turf at a concentration of about 150 mL per square meter.

In one aspect, the compositions and extracts disclosed herein are applied multiple times to the plants of interest. Further in this aspect, the compositions and extracts can be applied once per week, twice per week, or three times per week. In one aspect, the compositions and/or extracts are applied to the plants of interest twice per week.

In another aspect, the compositions and extracts disclosed herein can be applied alone or in combination with another product intended to enhance plant growth. In one aspect, the compositions and extracts disclosed herein are applied in combination with a fertilizer such as, for example, activated sewage sludge including, but not limited to, Hou-actinite, or another commercially-available fertilizer. In one aspect, the compositions and extracts disclosed herein are applied after fertilizer application, or can be applied before fertilizer application, or can be applied at the same time as fertilizer is applied to the plants of interest. In still another aspect, another compound such as, for example, chitosan, can be applied to the plants in addition to the compositions and extracts disclosed herein. In one aspect, the chitosan can be applied as a solution with a concentration of from about 0.01% to about 0.05% (w/v), or can be applied as a solution with a concentration of about 0.01, 0.02, 0.03, 0.04, or about 0.05%. In one aspect, the chitosan is applied as a solution with a concentration of about 0.02%. In another aspect, In one aspect, the chitosan can be applied at a concentration of about 50 to about 200 mL per square meter of, for example, turf, or can be applied at about 50, 75, 100, 125, 150, 175, or about 200 mL per square meter of turf, or a combination of any of the foregoing values, or a range encompassing any of the foregoing values. In one aspect, the chitosan is applied to turf at a concentration of 75 mL per square meter. In an alternative aspect, the chitosan is applied to turf at a concentration of 150 mL per square meter. In another aspect, the chitosan can be applied to the turf once per week, twice per week, or three times per week. In one aspect, the chitosan is applied twice per week. In still another aspect, the chitosan can be applied before the compounds and extracts disclosed herein, or after the compounds and extracts disclosed herein, or simultaneously with the compounds and extracts disclosed herein.

In any of the above aspects, application of the compounds and extracts disclosed herein leads to an increase in plant hormones and, in turn, improvement in properties such as earlier rooting, root anchoring or tension, root strength, root length, green coloration, nutrient availability, reduction of irrigation needs, prevention of erosion, and other desirable properties of plants. In another aspect, enhancing plant growth including magnesium uptake and production of chlorophyll a and/or chlorophyll b, or for other purposes, can be used to trap carbon dioxide from the atmosphere. Further in this aspect, trapping carbon dioxide is a method for reducing atmospheric pollution and/or lessening the impact of global warming. Demonstrations of carbon dioxide capture and atmospheric carbon dioxide reduction by treated plants are provided in the Examples.

### Additional Agricultural Applications

In an aspect, application of the compounds, compositions, and extracts disclosed herein to soil, croplands, and/or plants can induce or enhance regenerative agriculture instead of, or in addition to, traditional regenerative agriculture practices such as, for example, crop rotation, reduced plowing/tillage, regenerative livestock grazing practices, reduced use of artificial fertilizers, and the like.

In yet another aspect, application of the compounds, compositions, and extracts disclosed herein is useful for the production of functional foods, where a “functional food” is defined as a food product offering at least one benefit beyond or in addition to its nutritional value. Thus, for example, application of the compounds, compositions, and extracts can increase a macronutrient in a functional food (e.g. increasing amounts of fiber, protein, or both), or can add a vitamin or mineral to the functional food, or can reduce inflammation when the foods are consumed, or can help protect against or treat a disease when the foods are consumed.

### Anti-UV Applications

The genelight extracts produced herein may be applied to any material that may benefit from a reduction in exposure to UV radiation. The exact formulation of the extract plus any carriers can be adjusted based on the desired use. In one aspect, the extract is formulated with only non-toxic components if it is to be used on a human or animal or with another microorganism, such as in a fermentation process or on an agricultural product. In another aspect, the extract can be mixed with other substances to provide UV-protective properties to the overall composition. In still another aspect, if coated on the material to be protected, the extract itself can be covered with a further protective coating to project, for example, against mechanical wear and damage.

### Methods of Applying Extracts to Surfaces

In the case when the extract is applied to the surface of an article, it can be applied using techniques known in the art such spraying or coating. In other aspects, the extract can be intimately mixed with a substance or material that ultimately produces the article. For example, the extract can be mixed with molten glass so that the extract is dispersed throughout the final glass product.

In one aspect, the extract is formulated or applied in such a manner as to block approximately about 50%, about 60%, about 70%, about 80%, about 90%, about 95%, or about 99% of the UV radiation that encounters the extract, where any value can be a lower and upper end-point of a range (e.g., about 60% to about 95%, about 70% to about 80%). In a further aspect, the extract can also be formulated to block these percentages of particular UV wavelengths, or, more generally, to block these percentages of UVA, UVB, or UVC radiation.

### Uses of the Extracts

Extracts according to the present disclosure can be used for a variety of purposes. These purposes include, but are not limited to, the following:
(a) blocking UV radiation or other types of radiation;
(b) protecting human skin against damage and/or skin cancer induced by UV radiation or other types of radiation;
(c) protecting against side effects of radiation used in cancer treatments;
(d) protecting animals from deleterious effects of UV radiation or other radiation;
(e) protecting plastic, fiberglass, glass, rubber, or other solid surfaces from UV radiation or other radiation;
(f) providing a UV radiation screen or screen for other types of radiation;
(g) protecting astronauts and/or other persons or organisms as well as equipment during space trips;
(h) enhancement of industrial fermentation processes or other processes requiring energy by allowing the use of UV radiation in connection with the process to supply additional energy and thus to increase the ultimate energy-requiring output of the cells without substantially killing the fermenting organism;
(i) protection of experimentation, fermentation, biochemical, and/or biological processes under the presence of UV radiation, for example in extraterrestrial conditions such as on the moon or Mars; and
(j) protection of agricultural plants, particularly agricultural plants in which the revenue-producing part of the plant is above ground, such as fruits, vine vegetables, beans and peas, and leaf vegetables.

### Agricultural Applications

In one aspect, the genelight can be applied to an agricultural plant. In one aspect, the plant can be one that produces fruit or vegetable, such as, for example, a watermelon or a tomato. Further in this aspect, the extract can be applied during at least a part of the plant's growth to increase the amounts of one or more nutrients of the fruit or vegetable, such as a vitamin, mineral, or other recommended dietary component. In one specific aspect, the amount of lycopene can be increased (which may be accompanied by a decrease in carotene or other less-valuable nutrients formed by competing pathways). In another aspect, the amount of a flavor-enhancing component, such as glucose, can be increased. Further in this aspect, an increase in glucose can help protect against water loss.

In one aspect, the genelight extract can be applied for about 25%, about 50%, about 75%, about 90%, about 95%, or about 99% of the fruit's or vegetable's on-plant life, where any value can be a lower and upper endpoint of a range (e.g., about 25% to about 95%, about 50% to about 75%, etc.), and where the on-plant life includes the time span from the formation of a separate body that will constitute the fruit or vegetable (in some aspects, excepting flowers) until the fruit or vegetable is harvested. In one aspect, the extract can be first applied when the fruit or vegetable is sufficiently large to no longer be substantially protected from UV radiation by leaves.

In another aspect, the extract can first be applied five days, one week, or two weeks prior to harvest. Further in this aspect, application at this later stage can be particularly useful with fruits or vegetables in which an increase in a nutrient or flavor-enhancing component can be obtained by protecting the fruit or vegetable from UV radiation later in its on-plant life.

In one aspect, the genelight extract can be applied once or multiple times to each fruit or vegetable. In another aspect, it can be applied weekly, or it can be reapplied after the fruit or vegetable is exposed to rain or after a turning process. In another aspect, the agricultural plant can be another food crop that grows above ground and is exposed to natural UV radiation, wherein the agricultural product produced can be a fruit, leaf, seed, flower, grain, nut, stem, vegetable, or mushroom.

In another aspect, it is desirable for agricultural plants that do not produce parts typically consumed by humans to be protected from UV irradiation. In a further aspect, these other agricultural plants can includes sources of fibers such as, for example, cotton and linen (flax), of cork, of wood or lumber, of feedstocks for producing ethanol or biodiesel (including, but not limited to, sugar beet, sugarcane, cassava, sorghum, corn, wheat, oil palm, coconut, rapeseed, peanut, sunflower, soybean, and the like), of animal feedstocks or fodder, or of decorative or horticultural plants.

In one aspect, any part of the plant can be coated with the genelight extract, including, but not limited to, the part of the plant that is collected during harvest. In an alternative aspect, the harvested part of the plant is not coated, but another part can be coated with the extracts disclosed herein. In addition to the aspects already described, in one aspect, coating a plant with the extracts described herein can prolong the life of the plant, increase production capacity of a desired product, can increase the growth rate of the plant relative to an untreated plant of the same type, can increase production of a desired metabolite that might otherwise decrease due to UV-induced stress, can increase yield of a crop of such plants, and the like.

In a further aspect, application can be accomplished with a commercial sprayer. In another aspect, application can be only on the upper portions of the fruit or vegetable, which are exposed to substantially greater amounts of UV radiation than the lower portions of the fruit or vegetable.

### Cosmetics and Pharmaceutical Compositions Containing the Extracts

In another aspect, provided herein is a pharmaceutical composition containing the genelight extracts described herein. In one aspect, the pharmaceutical composition can be applied to a subject, wherein the subject is exposed to radiation. In one aspect, the radiation is applied as a strategy to treat cancer. In another aspect, the pharmaceutical composition is used to prevent radiation-induced cellular and DNA damage. In another aspect, dosage ranges of the extract in the pharmaceutical composition can vary from about 0.01 g extract/mL of pharmaceutical composition to about 1 g extract/mL of pharmaceutical composition, or can be about 0.01 g extract/mL, about 0.02 g extract/mL, about 0.025 g extract/mL, about 0.05 g extract/mL, about 0.075 g extract/mL, or about 1 g extract/mL of pharmaceutical composition, where any value can be a lower and upper endpoint of a range (e.g., about 0.01 g extract/mL to about 0.075 g extract/mL, about 0.025 g extract/mL to about 0.05 g extract/mL, etc.). In an alternative aspect, provided herein is a cosmetic composition containing the genelight extracts produced herein. Further in this aspect, the cosmetic composition can be a cleanser, lotion, cream, shampoo, hair treatment, makeup, lip treatment, nail treatment, or related composition. In still a further aspect, the compositions containing the extracts can have both pharmaceutical and cosmetic applications. In yet another aspect, the compositions containing the extracts can be used in veterinary medicine.

The cosmetic compositions can be formulated in any physiologically acceptable medium typically used to formulate topical compositions. The cosmetic compositions can be in any galenic form conventionally used for a topical application such as, for example, in the form of dispersions of aqueous gel or lotion type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or alternatively multiple emulsions (W/O/VV or O/W/O), microemulsions, vesicular dispersions of ionic and/or non-ionic type, or wax/aqueous phase dispersions. These compositions are prepared according to the usual methods.

The cosmetic compositions can also contain one or more additives commonly used in the cosmetics field, such as emulsifiers, preservatives, sequestering agents, fragrances, thickeners, oils, waxes or film-forming polymers. In one aspect, in any of the above scenarios, the pharmaceutical, cosmetic, or veterinary composition also includes additional UV-protective compounds or UV-blocking agents such as, for example, zinc oxide, titanium dioxide, carotenoids, oxybenzone, octinoxate, homosalate, octisalate, octocrylene, avobenzone, or a combination thereof.

In one aspect, the composition is a sunscreen. A sunscreen can be formulated with any of the extracts produced herein. In addition to the extract, the sunscreen in certain aspects can be formulated with one or more UV-protective compounds or UV-blocking agents listed above.

The sunscreen can be formulated as a paste, lotion, cream, aerosol, or other suitable formulations for topical use. In certain aspects, the sunscreen can be formulated as a transparent composition.

In one aspect, the cosmetic composition can be a film composed of the genelight extracts produced herein that can be directly applied to the skin. For example, the film can be composed of a biocompatible material such as a protein or oligonucleotide, where the extract is coated on one or more surfaces of the film or, in the alternative dispersed throughout the film. For example, the film can be composed of DNA. In this application, the films can be used as a wound covering and provide protection from UV photodamage. The films can also be prepared so that they are optically transparent. Here, it is possible to view the wound without removing the covering and exposing the wound. The films can also include other components useful in cosmetic applications such as, for example, compounds to prevent or reduce wrinkles.

In one aspect, the pharmaceutical, cosmetic, or veterinary compositions described herein are applied to subjects. In one aspect, the subject is a human, another mammal, or a bird. In a further aspect, the mammal is a pet such as a dog or cat or is livestock such as horses, goats, cattle, sheep, and the like. In an alternative aspect, the bird is a pet bird or is poultry such as, for example, a chicken or turkey. In any of these aspects, the compositions can be applied to skin, fur, feathers, wool, hooves, horns, or hair as appropriate and applicable.

In a related aspect, the compositions and extracts disclosed herein and/or the pharmaceutical, cosmetic, or veterinary compositions disclosed herein are applied to isolated human or animal cells. Further in this aspect, the compositions and extracts and/or the pharmaceutical, cosmetic, or veterinary compositions disclosed herein can be applied to the isolated human or animal cells in any desired volume ratio such as, for example, from about 1:7 to about 7:1, or about 1:7, 2:5, 5:4, 5:2, or about 7:1. In one aspect, the compositions or extracts disclosed herein are applied to human fibroblast cells in culture at a volume ratio of about 5:4.

Further in this aspect, the compositions and extracts disclosed herein protect the isolated human or animal cells from UV-induced cell death. Still further in this aspect, this protection can be assessed by known means such as, for example, staining with trypan blue and counting living and/or dead cells under a microscope, or by assessing total amount of genomic DNA present in the sample by a means such as, for example, gel electrophoresis followed by quantification of stain intensity.

### Paints, Inks, Dyes, and Stains

In another aspect, provided herein is a paint, dye, stain, or ink containing the genelight extracts disclosed herein. In one aspect, there are several benefits to having a paint that is resistant to UV irradiation. In a further aspect, imparting UV resistance to a paint slows or stops photodegradation, bleaching, or color fading. In another aspect, a paint with UV resistance prevents chemical modification of exposed paint surfaces. Further in this aspect, chemical modification of exposed paint surfaces includes change in finish, structural changes in binders, flaking, chipping, and the like. In one aspect, the paint provided herein resists these changes.

### Articles Incorporating the Genelight Extracts

In still another aspect, provided herein is an article coated with the genelight extracts disclosed herein. In one aspect, the article is made of glass, plastic, metal, wood, fabric, or any combination thereof. In one aspect, the article is a construction material such as, for example, steel, concrete or cement, brick, wood, window or door glass, fiberglass, siding, wallboard, a flooring material, masonry, mortar, grout, stone, artificial stone, stucco, shingles, roofing materials, and the like. In an alternative aspect, the material is an aeronautical or aerospace material such as, for example, the metal or metal alloy body of an aircraft or spacecraft, paint on the body of an aircraft or spacecraft, glass windows on an aircraft or spacecraft, carbon fiber composite, titanium or aluminum, a ceramic heat absorbing tile, and the like. In still another aspect, the article is a fabric article such as, for example, clothing, drapes, outdoor upholstery, a tert or outdoor pavilion, a flag or banner, or the like. In another aspect, the extract can be applied to the article to fine artwork, solid pieces (e.g., vases), and historical documents in order to preserve them. In another aspect, the extract can be applied to outdoor signs such as highway billboards and advertising.

In other aspects, the genelight extract can be incorporated within or throughout the article. In one aspect, the extract can be mixed with molten glass to produce glass article that are UV resistant such as, for example, sunglasses, car windshields, window glass, and eyeglasses. In another aspect, the glass article can be a bottle for storing a beverage or food container in order to increase the shelf-life of the beverage or food. It is contemplated that the extract can be applied externally to the glass articles as well.

In another aspect, the genelight extract can be mixed with fiberglass or plastics in order to reduce negative effects to aircraft, watercraft, boats, jet skis, decking, house siding, motor homes, sunroofs, and moon roofs that are constantly exposed to UV radiation. It is contemplated that the extract can be applied externally to the fiberglass or plastic articles as well.

In another aspect, the genelight extract can be mixed with rubber, silicon, or latex used to make a variety of articles such as water hoses, tires, and the like. It is contemplated that the extract can be applied externally to the rubber, silicone, or latex articles as well.

In another aspect, the genelight extract can be mixed with foams used to make a variety of articles such as automotive dashboard padding, seat cushions, and the like. It is contemplated that the extract can be applied externally to the foam articles as well.

In another aspect, the genelight extracts described herein can be incorporated into an optical film. In one aspect, the extract is applied to at least one surface of the film. In another aspect, the extract can be dispersed throughout the film. The film can be transparent, translucent or opaque. The film can be composed of, but not limited to, polyolefin resin, such as polyethylene (PE) or polypropylene (PP); polyester resin, such as polyethylene terephthalate (PET); polyacrylate resin, such as polymethyl (meth)acrylate (PMMA); polycarbonate resin; polyurethane resin or a mixture thereof. The optical film can be applied to any substrate where it is desirable to reduce or prevent UV exposure or damage. For example, the optical film can be applied to windows to reduce or prevent UV radiation from entering a structure (e.g., building, vehicle, etc.).

### Methods for Reducing or Preventing Exposure to UV Radiation

In another aspect, provided herein is a method of reducing or preventing the exposure of an item to UV radiation by applying the genelight extracts described herein to the item or incorporating the extract within/throughout the article. Further in this aspect, “reducing” is defined relative to an untreated control. That is, if two like items are exposed to equal amounts of UV radiation for an equal amount of time, but one has been treated with the UV-resistant extracts and the other has not, and some objective response is measured (e.g., color fading, structural degradation, plant size or yield, etc.), the treated item will appear to have been exposed to a lower amount of UV (for example, the color of the treated item will have faded less and will remain closer to the original, or a treated plant will appear larger and more vigorous and will have a greater yield, etc.). In some aspects, treatment with the extracts disclosed herein will prevent UV exposure from occurring. As used herein, “prevent” indicates that a treated item will not be affected, changed, or altered by UV exposure.

In one aspect, the genelight extract blocks from about 50% to about 100% of UV radiation from contacting the item. Further in this aspect, the extract blocks about 50%, about 55%, about 60%, about 65%, about 70%, about 75%, about 80%, about 85%, about 90%, about 95%, about 99%, or about 100% of UV radiation from contacting the item, where any value can be a lower and upper endpoint of a range (e.g., about 50% to about 90%, about 60% to about 80%, etc.). In another aspect, the extract blocks from about 50% to about 100% of longwave UV radiation from contacting the item. Further in this aspect, the extract blocks about 50%, about 55%, about 60%, about 65%, about 70%, about 75%, about 80%, about 85%, about 90%, about 95%, about 99%, or about 100% of longwave UV radiation from contacting the item, where any value can be a lower and upper endpoint of a range (e.g., about 50% to about 90%, about 60% to about 80%, etc.). In one aspect, the extract blocks from about 50% to 100% of shortwave UV radiation from contacting the item. Further in this aspect, the extract blocks about 50%, about 55%, about 60%, about 65%, about 70%, about 75%, about 80%, about 85%, about 90%, about 95%, about 99%, or about 100% of shortwave UV radiation from contacting the item, where any value can be a lower and upper endpoint of a range (e.g., about 50% to about 90%, about 60% to about 80%, etc.).

Depending upon the application, the genelight extract can prevent or reduce damage cause by UV radiation from limited to extended periods of time. By varying the amount of extract that is applied as well as the number of times the extract is applied, the degree of UV protection can be varied. In certain aspects, it may be desirable for the article to be protected from UV damage for a short period of time then subsequently biodegrade.

### Preventing or Reducing the Growth of Barnacles

In another aspect, the genelight extracts produced herein can be used to reduce or prevent the growth of barnacles on boats and other water vehicles. In one aspect, the extract can be admixed with a paint that is typically applied to water vehicles, where the paint also includes chitosan. In one aspect, the chitosan can be acetylated to a specific degree of acetylation in order to enhance tissue growth during culturing as well as metabolite production. In one aspect, the chitosan is from about 60% to about 100%, or about 60%, about 65%, about 70%, about 75%, about 80%, about 85%, about 90%, about 95%, about 99%, or about 100%, where any value can be a lower and upper endpoint of a range (e.g., about 60% to about 90%, about 70% to about 80%, etc.). In one aspect, chitosan isolated from the shells of crab, shrimp, lobster, and/or krill is useful herein. The molecular weight of the chitosan can vary, as well. For example, the chitosan comprises about 3, 4, 5, 6, 7, 8, 9, 10, 11, 12, 13, 14, 15, 16, 17, 18, 19, or 20 glucosamine units and/or N-acetylglucosamine units, where any value can be a lower and upper endpoint of a range (e.g., 3 to about 19, 5 to 7, etc.).

Protection of Microbial Cultures from the Effects of UV Radiation

In one aspect, the compositions and extracts disclosed herein can be applied to cultures of commercially-important microorganisms. Further in this aspect, the compositions and extracts disclosed herein offer protection from UV radiation to the microorganisms. In one aspect, the microorganisms are used for the production of industrially-important enzymes or chemical compounds such as, for example, Acetobacter aceti (source of acetic acid), Aspergillus niger (source of citrus acid), any microorganism that produces enzymes such as, for example, cellulases, amylase, palatase, lipozyme, lipase, lipopan F, xylose isomerase, resinase, penicillin amidase, or amidase. In another aspect, the microorganisms are important for fermentation in food production, including, but not limited to, Saccharomyces cerevisiae for the production of bread and for alcoholic fermentation, bacteria such as Streptococcus thermophiles, Lactobacillus delbrueckii, and other lactobacilli and bifidobacteria important in the production of yogurt, skyr, kefir, and other fermented dairy products, Lactococcus species and Propionibacter shermani used in the production of some cheeses, Clostridium butyricum useful in the process of retting of jute, hemp, and/or flax, microorganisms used for water treatment and/or sewage treatment, experimental microorganisms useful in molecular biology, genetic engineering, and other laboratory uses including, but not limited to, E. coli, B. subtilis, and the like, microorganisms useful in the production of B vitamins and other vitamins, microorganisms such as Bacillus thuringiensis useful for agricultural pest control, and the like.

In one aspect, the cultures and extracts disclosed herein can be mixed in any proportion with cultures of commercially important microorganisms for the purpose of providing protection from UV-irradiation to the commercially important microorganisms. In one aspect, the cultures and extracts can be applied in a volume ratio of from 1:7 to 7:1 to cultures of the commercially important microorganisms, or can be applied in a ratio of about 1:7, 2:5, 3:4, 4:3, 5:2, 7:1, or a combination of any of the foregoing values, or a range encompassing any of the foregoing values. In a further aspect, the cultures and extracts are applied in a ratio of 5:2 to cultures of commercially important microorganisms.

In another aspect, treatment with the cultures and extracts disclosed herein protects the commercially important microorganisms from negative effects of UV radiation for a period of from 10 minutes to 2 hours, or for about 10 minutes, 15 minutes, 20 minutes, 30 minutes, 1 hour, 90 minutes, or about 2 hours, or a combination of any of the foregoing values or a range encompassing any of the foregoing values. In one aspect, successful protection from negative effects of UV radiation can be assessed by culturing cells of the commercially important microorganism in a Petri dish and counting colonies, or by any other common means for assessing cell survival, with higher numbers of surviving cells being indicative of successful UV protection. In any of the above aspects, the cultures and extracts disclosed herein are biocompatible and do not harm the commercially important microorganisms.

A “polyactive carbohydrate” as used herein refers to an extract from a device such as those disclosed and described in PCT patent application publications WO 2019/055456 and WO 2021/071851; U.S. Pat. No. 10,995,353; and US patent application publication 2021/0261997. In one aspect, a device for producing a polyactive carbohydrate includes genes encoding one or more of the following proteins: chitin synthase, chitosanase, chitin deacetylase, lipase, chitin synthase regulatory factor CHR1, transglycosylase, dehydrogenase, (1→3),(1→4)-β-glucan synthase; transglycosylase, or any combination thereof. In another aspect, exemplary DNA sequences for vectors for producing a polyactive carbohydrate include those identified by SEQ ID NOs. 14-21 or having 70% or more homology thereto. In still another aspect, the device for producing a polyactive carbohydrate can be transfected into a microorganism such as, for example, E. coli, S. cerevisiae, or another bacterial or fungal species by methods described herein. In still another aspect, the vector can be pYES2, pETDuet-1, or pBSK.

An “anti-UV extract” as used herein refers to an extract from a device such as those disclosed and described in PCT patent application publications WO 2018/213526, WO 2020/257524, and WO 2019/165163; U.S. Pat. Nos. 11,639,505 and 11,692,192; and US patent application publication 2023/0340497. In one aspect, a device for producing a an anti-UV extract includes genes encoding one or more of the following proteins: hexokinase, a heat shock protein such as, for example, HSP70, alcohol dehydrogenase, transferrin, zinc oxidase, iron oxidase, flavonol synthase, zinc-related protein/oxidase, silicatein, silaffin, lipase, phosphoribulokinase, α-actin, RuBisCO large subunit, tonB, or any combination thereof. In another aspect, exemplary DNA sequences for vectors for producing an anti-UV extract include those identified by SEQ ID NOs. 7-13 or having 70% or more homology thereto. In still another aspect, the device for producing an anti-UV extract can be transfected into a microorganism such as, for example, E. coli, S. cerevisiae, or another bacterial or fungal species by methods described herein. In still another aspect, the vector can be pYES2 or pBSK.

A “genelight device” or “genelight extract” as used herein refers both to the disclosed devices and extracts, as well as those devices and extracts disclosed and described in PCT patent application publication WO 2021/046464 and US patent application publication 2022/0183301. In a further aspect, different genelight devices can be cultured in the same culture medium and same culture vessel to produce a genelight extract. In an alternative aspect, different genelight devices can be cultured separately and the resulting extracts mixed together in any proportion. In one aspect, a genelight device or a device for producing a genelight extract includes genes encoding one or more of the following proteins: a heat shock protein such as, for example, HSP70, RuBisCO large subunit 1, tonB, hydrogenase, P-type ATPase, dehydrogenase, or any combination thereof. In another aspect, exemplary DNA sequences for vectors for producing an anti-UV extract include those identified by SEQ ID NOs. 26-29 or having 70% or more homology thereto. In still another aspect, the genelight device or the device for producing genelight extract can be transfected into a microorganism such as, for example, E. coli, S. cerevisiae, or another bacterial or fungal species by methods described herein. In still another aspect, the vector can be pYES2 or pBAD.

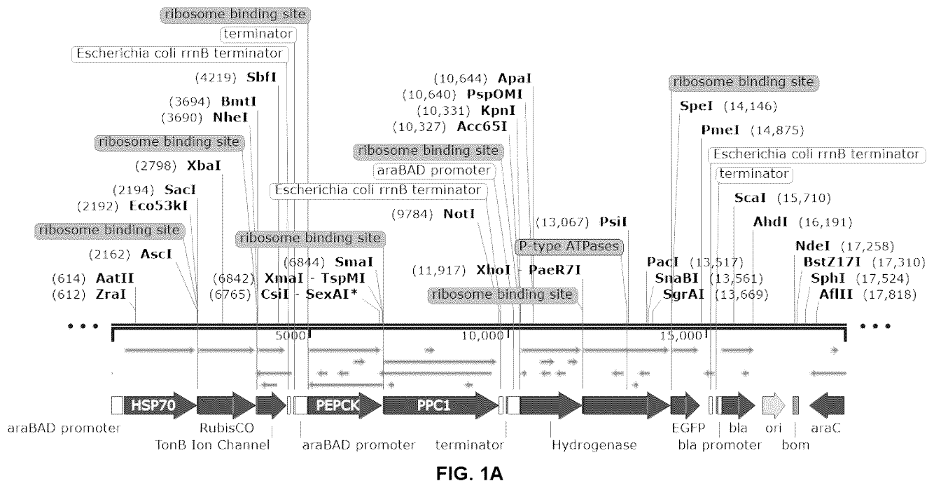

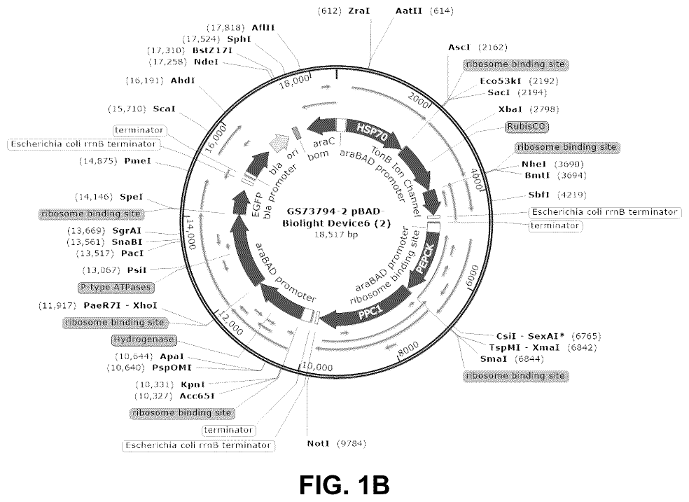

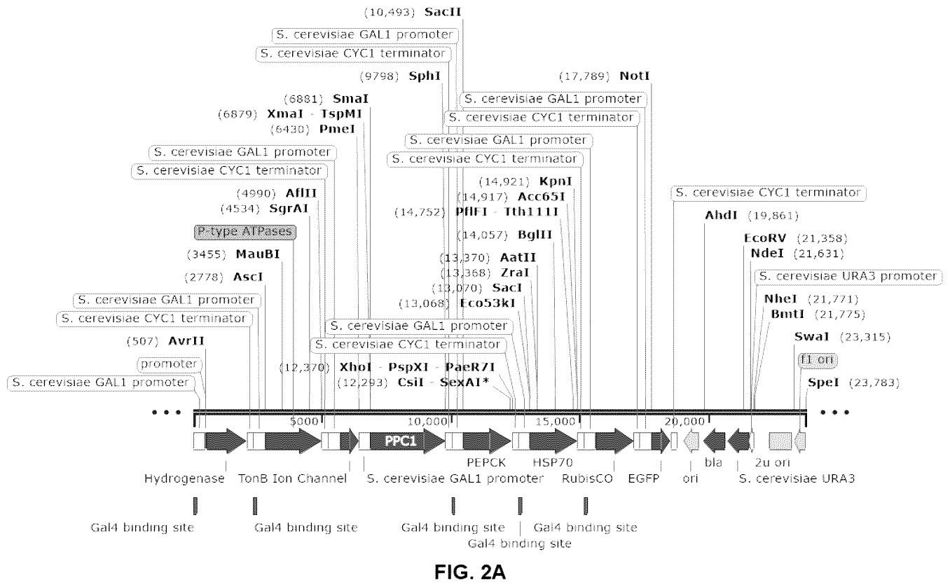

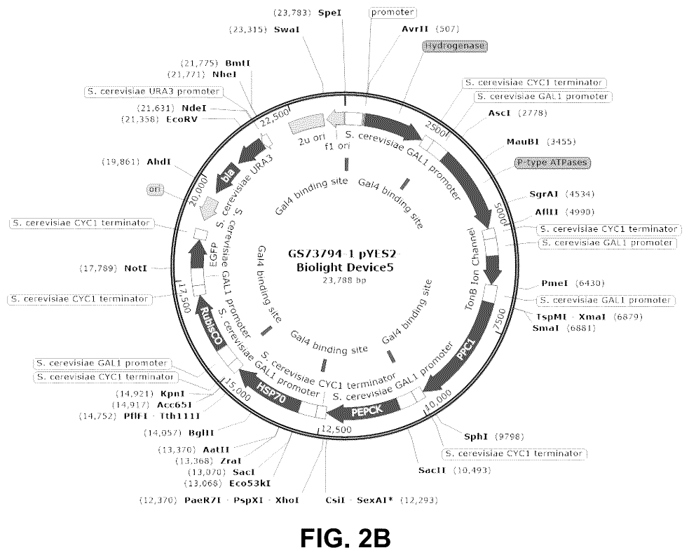

In one aspect, co-culturing a disclosed device with an anti-UV device, a polyactive carbohydrate device, or another genelight device can produce an extract or composition with increased efficacy for any of the applications disclosed herein. In another aspect, separately culturing one or more of a disclosed device, an anti-UV device, a polyactive carbohydrate device, or another genelight device can result in separate compositions or extracts that can be mixed in any desired proportion or combination in order to produce an extract or composition with increased efficacy for any of the applications disclosed herein. In still another aspect, extracts produced separately by biological devices in bacterial and yeast cultures can be mixed together for producing treatments with maximum efficacy. In one non-limiting example, an extract from a bacterial device such as the one depicted in FIGS. 1A-1B and an extract from a fungal device such as the one depicted in FIGS. 2A-2B can be mixed together in any proportion, and/or can be mixed separately with another device or extract for use in the methods described herein (e.g. genelight, anti-UV, or polyactive carbohydrate)

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### ASPECTS

The present disclosure can be described in accordance with the following numbered aspects, which should not be confused with the claims.

Aspect 1. A DNA construct comprising the following genetic components:
(a) a gene that encodes a heat shock protein;
(b) a gene that encodes RuBisCO large subunit 1;
(c) a gene that encodes tonB;
(d) a gene that encodes phosphoenol pyruvate carboxykinase;
(e) a gene that encodes phosphoenol pyruvate carboxylase;
(f) a gene that encodes a hydrogenase; and
(g) a gene that encodes a p-type ATPase.

Aspect 2. The DNA construct of aspect 1, wherein the gene that encodes the heat shock protein has SEQ ID NO. 1 or at least 70% homology thereto.

Aspect 3. The DNA construct of aspect 1, wherein the gene that encodes RuBisCO large subunit 1 has SEQ ID NO. 2 or at least 70% homology thereto.

Aspect 4. The DNA construct of aspect 1, wherein the gene that encodes tonB has SEQ ID NO. 3 or at least 70% homology thereto.

Aspect 5. The DNA construct of aspect 1, wherein the gene that encodes phosphoenol pyruvate carboxykinase has SEQ ID NO. 4 or at least 70% homology thereto.

Aspect 6. The DNA construct of aspect 1, wherein the gene that encodes phosphoenol pyruvate carboxylase has SEQ ID NO. 5 or at least 70% homology thereto.

Aspect 7. The DNA construct of aspect 1, wherein the gene that encodes the hydrogenase has SEQ ID NO. 6 or at least 70% homology thereto.

Aspect 8. The DNA construct of aspect 1, wherein the gene that encodes the p-type ATPase has SEQ ID NO. 7 or at least 70% homology thereto.

Aspect 9. The DNA construct of aspect 1, wherein the construct further comprises at least one promoter.

Aspect 10. The DNA construct of aspect 9, wherein the at least one promoter is a T3 promoter, a T7 promoter, an iron promoter, an araBAD promoter, a GAL1 promoter, or any combination thereof.

Aspect 11. The DNA construct of aspect 10, wherein the at least one promoter is an araBAD promoter, and the araBAD promoter is positioned before the gene that encodes the heat shock protein, the gene that encodes RuBisCO large subunit 1, the gene that encodes tonB, the gene that encodes phosphoenol pyruvate carboxy kinase, the gene that encodes phosphoenol pyruvate carboxylase, the gene that encodes the hydrogenase, the gene that encodes the p-type ATPase, or any combination thereof.

Aspect 12. The DNA construct of aspect 1, wherein the DNA construct further comprises a gene that confers resistance to an antibiotic.

Aspect 13. The DNA construct of aspect 12, wherein the antibiotic comprises tetracycline, neomycin, kanamycin, ampicillin, hygromycin, chloramphenicol, amphotericin B, bacitracin, carbapenem, cephalosporin, ethambutol, fluoroquinolones, isoniazid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

Aspect 14. The DNA construct of aspect 1, wherein the DNA construct further comprises at least one terminator.

Aspect 15. The DNA construct of aspect 14, wherein the at least one terminator is an rrnB terminator.

Aspect 16. The DNA construct of aspect 1, wherein the DNA construct further comprises a reporter protein.

Aspect 17. The DNA construct of aspect 16, wherein the reporter protein is a fluorescent reporter protein.

Aspect 18. The DNA construct of aspect 17, wherein the fluorescent reporter protein is a red fluorescent protein, a cyan fluorescent protein, a green fluorescent protein, or a yellow fluorescent protein.

Aspect 19. The DNA construct of aspect 18, wherein the fluorescent reporter protein is a green fluorescent protein.

Aspect 20. The DNA construct of aspect 19, wherein the green fluorescent protein has SEQ ID NO. 8 or has at least 70% homology thereto.

Aspect 21. The DNA construct of aspect 1, wherein the construct comprises from 5′ to 3′ the following genetic components in the following order: (a) a gene that encodes a heat shock protein; (b) a gene that encodes RuBisCO large subunit 1; (c) a gene that encodes tonB; (d) a gene that encodes phosphoenol pyruvate carboxykinase; (e) a gene that encodes phosphoenol pyruvate carboxylase; (f) a gene that encodes a hydrogenase; and (g) a gene that encodes a p-type ATPase.

Aspect 22. The DNA construct of aspect 1, wherein the construct comprises from 5′ to 3′ the following genetic components in the following order: (a) a gene that encodes a heat shock protein having SEQ ID NO. 1 or at least 70% homology thereto; (b) a gene that encodes RuBisCO large subunit 1 having SEQ ID NO. 2 or at least 70% homology thereto; (c) a gene that encodes tonB having SEQ ID NO. 3 or at least 70% homology thereto; (d) a gene that encodes phosphoenol pyruvate carboxykinase having SEQ ID NO. 4 or at least 70% homology thereto; (e) a gene that encodes phosphoenol pyruvate carboxylase having SEQ ID NO. 5 or at least 70% homology thereto; (f) a gene that encodes a hydrogenase having SEQ ID NO. 6 or at least 70% homology thereto; and (g) a gene that encodes a p-type ATPase having SEQ ID NO. 7 or at least 70% homology thereto.

Aspect 23. The DNA construct of aspect 1, wherein the construct comprises from 5′ to 3′ the following genetic components in the following order: (a) a gene that encodes a heat shock protein; (b) a gene that encodes RuBisCO large subunit 1; (c) a gene that encodes tonB; (d) an rrnB terminator; (e) an araBAD promoter; (f) a gene that encodes phosphoenol pyruvate carboxykinase; (g) a gene that encodes phosphoenol pyruvate carboxylase; (h) an rrnB terminator; (i) an araBAD promoter; (j) a gene that encodes a hydrogenase; and (k) a gene that encodes a p-type ATPase.

Aspect 24. The DNA construct of aspect 1, wherein the construct comprises from 5′ to 3′ the following genetic components in the following order: (a) a gene that encodes a heat shock protein having SEQ ID NO. 1 or at least 70% homology thereto; (b) a gene that encodes RuBisCO large subunit 1 having SEQ ID NO. 2 or at least 70% homology thereto; (c) a gene that encodes tonB having SEQ ID NO. 3 or at least 70% homology thereto; (d) an rrnB terminator; (e) an araBAD promoter; (f) a gene that encodes phosphoenol pyruvate carboxykinase having SEQ ID NO. 4 or at least 70% homology thereto; (g) a gene that encodes phosphoenol pyruvate carboxylase having SEQ ID NO. 5 or at least 70% homology thereto; (h) an rrnB terminator; (i) an araBAD promoter; (j) a gene that encodes a hydrogenase having SEQ ID NO. 6 or at least 70% homology thereto; and (k) a gene that encodes a p-type ATPase having SEQ ID NO. 7 or at least 70% homology thereto.

Aspect 25. The DNA construct of aspect 1, wherein the DNA construct has SEQ ID NO. 9.

Aspect 26. A vector comprising the DNA construct of aspect 1.

Aspect 27. The vector of aspect 26, wherein the vector is a plasmid.

Aspect 28. The vector of aspect 27, wherein the plasmid is pWLneo, pSV2cat, pOG44, pXT1, pSG, pSVK3, pBSK, pBSKII, pYES, pYES2, pET, pBAD, pUC, or pUC19.

Aspect 29. The vector of aspect 28, wherein the plasmid is pBAD.

Aspect 30. A biological device comprising host cells transformed with the DNA construct in any one of aspects 1-25.

Aspect 31. The device of aspect 30, wherein the host cells comprise fungi or bacteria.

Aspect 32. The device of aspect 31, wherein the bacteria comprise Escherichia coli.

Aspect 33. A DNA construct comprising the following genetic components:
(a) a gene that encodes a hydrogenase;
(b) a gene that encodes a p-type ATPase;
(c) a gene that encodes phosphoenol pyruvate carboxylase;
(d) a gene that encodes phosphoenol pyruvate carboxykinase;
(e) a gene that encodes tonB;
(f) a gene that encodes a heat shock protein; and
(g) a gene that encodes RuBisCO large subunit 1.

Aspect 34. The DNA construct of aspect 33, wherein the gene that encodes the hydrogenase has SEQ ID NO. 6 or at least 70% homology thereto.

Aspect 35. The DNA construct of aspect 33, wherein the gene that encodes the p-type ATPase has SEQ ID NO. 7 or at least 70% homology thereto.

Aspect 36. The DNA construct of aspect 33, wherein the gene that encodes phosphoenol pyruvate carboxylase has SEQ ID NO. 5 or at least 70% homology thereto.

Aspect 37. The DNA construct of aspect 33, wherein the gene that encodes phosphoenol pyruvate carboxykinase has SEQ ID NO. 4 or at least 70% homology thereto.

Aspect 38. The DNA construct of aspect 33, wherein the gene that encodes the tonB has SEQ ID NO. 3 or at least 70% homology thereto.

Aspect 39. The DNA construct of aspect 33, wherein the gene that encodes the heat shock protein has SEQ ID NO. 1 or at least 70% homology thereto.

Aspect 40. The DNA construct of aspect 33, wherein the gene that encodes the RuBisCO large subunit 1 has SEQ ID NO. 2 or at least 70% homology thereto.

Aspect 41. The DNA construct of aspect 33, wherein the construct further comprises at least one promoter.

Aspect 42. The DNA construct of aspect 41, wherein the at least one promoter is a T3 promoter, a T7 promoter, an iron promoter, a GAL1 promoter, or any combination thereof.

Aspect 43. The DNA construct of aspect 42, wherein the at least one promoter is a GAL1 promoter, and the GAL1 promoter is positioned before the gene that encodes the hydrogenase; the gene that encodes the p-type ATPase, the gene that encodes phosphoenol pyruvate carboxylase, the gene that encodes phosphoenol pyruvate carboxykinase; the gene that encodes tonB; the gene that encodes the heat shock protein; the gene that encodes RuBisCO large subunit 1, or any combination thereof.

Aspect 44. The DNA construct of aspect 33, wherein the DNA construct further comprises a gene that confers resistance to an antibiotic.

Aspect 45. The DNA construct of aspect 44, wherein the antibiotic comprises tetracycline, neomycin, kanamycin, ampicillin, hygromycin, chloramphenicol, amphotericin B, bacitracin, carbapenem, cephalosporin, ethambutol, fluoroquinolones, isoniazid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

Aspect 46. The DNA construct of aspect 33 wherein the DNA construct further comprises at least one terminator.

Aspect 47. The DNA construct of aspect 46, wherein the at least one terminator is a CYC1 terminator.

Aspect 48. The DNA construct of aspect 33, wherein the DNA construct further comprises a reporter protein.

Aspect 49. The DNA construct of aspect 48 wherein the reporter protein is a fluorescent reporter protein.

Aspect 50. The DNA construct of aspect 49, wherein the fluorescent reporter protein is a red fluorescent protein, a cyan fluorescent protein, a green fluorescent protein, or a yellow fluorescent protein.

Aspect 51. The DNA construct of aspect 50, wherein the fluorescent reporter protein is a green fluorescent protein.

Aspect 52. The DNA construct of aspect 51, wherein the green fluorescent protein is SEQ ID NO. 8 or has at least 70% homology thereto.

Aspect 53. The DNA construct of aspect 33, wherein the construct comprises from 5′ to 3′ the following genetic components in the following order: (a) a gene that encodes a hydrogenase; (b) a gene that encodes a p-type ATPase; (c) a gene that encodes phosphoenol pyruvate carboxylase; (d) a gene that encodes phosphoenol pyruvate carboxykinase; (e) a gene that encodes tonB; (f) a gene that encodes a heat shock protein; and (g) a gene that encodes RuBisCO large subunit 1.

Aspect 54. The DNA construct of aspect 33, wherein the construct comprises from 5′ to 3′ the following genetic components in the following order: (a) a gene that encodes a hydrogenase having SEQ ID NO. 6 or at least 70% homology thereto; (b) a gene that encodes a p-type ATPase having SEQ ID NO. 7 or at least 70% homology thereto; (c) a gene that encodes phosphoenol pyruvate carboxylase having SEQ ID NO. 5 or at least 70% homology thereto; (d) a gene that encodes phosphoenol pyruvate carboxykinase having SEQ ID NO. 4 or at least 70% homology thereto; (e) a gene that encodes tonB having SEQ ID NO. 3 or at least 70% homology thereto; (f) a gene that encodes a heat shock protein having SEQ ID NO. 1 or at least 70% homology thereto; and (g) a gene that encodes RuBisCO large subunit 1 having SEQ ID NO. 2 or at least 70% homology thereto.

Aspect 55. The DNA construct of aspect 33, wherein the construct comprises from 5′ to 3′ the following genetic components in the following order: (a) a gene that encodes a hydrogenase; (b) a CYC1 terminator; (c) a GAL1 promoter; (d) a gene that encodes a p-type ATPase; (e) a CYC1 terminator; (f) a GAL1 promoter; (g) a gene that encodes phosphoenol pyruvate carboxylase; (h) a CYC1 terminator; (i) a GAL1 promoter; (j) a gene that encodes phosphoenol pyruvate carboxykinase; (k) a CYC1 terminator; (l) a GAL1 promoter; (m) a gene that encodes tonB; (n) a CYC1 terminator; (o) a GAL1 promoter; (p) a gene that encodes a heat shock protein; (q) a CYC1 terminator; (r) a GAL1 promoter; and (s) a gene that encodes RuBisCO large subunit 1.

Aspect 56. The DNA construct of aspect 33, wherein the construct comprises from 5′ to 3′ the following genetic components in the following order: (a) a gene that encodes a hydrogenase having SEQ ID NO. 6 or at least 70% homology thereto; (b) a CYC1 terminator; (c) a GAL1 promoter; (d) a gene that encodes a p-type ATPase having SEQ ID NO. 7 or at least 70% homology thereto; (e) a CYC1 terminator; (f) a GAL1 promoter; (g) a gene that encodes phosphoenol pyruvate carboxylase having SEQ ID NO. 5 or at least 70% homology thereto; (h) a CYC1 terminator; (i) a GAL1 promoter; (j) a gene that encodes phosphoenol pyruvate carboxykinase having SEQ ID NO. 4 or at least 70% homology thereto; (k) a CYC1 terminator; (l) a GAL1 promoter; (m) a gene that encodes tonB having SEQ ID NO. 3 or at least 70% homology thereto; (n) a CYC1 terminator; (o) a GAL1 promoter; (p) a gene that encodes a heat shock protein having SEQ ID NO. 1 or at least 70% homology thereto; (q) a CYC1 terminator; (r) a GAL1 promoter; and (s) a gene that encodes RuBisCO large subunit 1 having SEQ ID NO. 2 or at least 70% homology thereto.

Aspect 57. The DNA construct of aspect 33, wherein the DNA construct has SEQ ID NO. 10.

Aspect 58. A vector comprising the DNA construct of aspect 33.

Aspect 59. The vector of aspect 58, wherein the vector is a plasmid.

Aspect 60. The vector of aspect 59 wherein the plasmid is pWLneo, pSV2cat, pOG44, pXT1, pSG, pSVK3, pBSK, pBSKII, pYES, pYES2, pET, pBAD, pUC, or pUC19.

Aspect 61. The vector of aspect 60, wherein the plasmid is pYES2.

Aspect 62. A biological device comprising host cells transformed with the DNA construct in any one of aspects 33-57

Aspect 63. The biological device of aspect 62, wherein the host cells comprise fungi or bacteria.

Aspect 64. The biological device of aspect 63, wherein the fungi comprise Saccharomyces cerevisiae.

Aspect 65. An extract produced by culturing the biological device of any one of aspects 30-32 or 62-64 in a culture medium.

Aspect 66. The extract of aspect 65, wherein the extract is formulated as a liquid, a slurry, a powder, a pellet, or a mixture thereof.

Aspect 67. A battery comprising an electrolyte, wherein the electrolyte comprises the extract of aspect 65.

Aspect 68. An article or device comprising the extract of aspect 65.

Aspect 69. The article or device of aspect 68, wherein the article or device comprises a standalone light, an indicator light on a larger device, a lighted panel on a larger device, a computer, a tablet, a smartphone, a medical device, a portable heating device, a portable cooling device, a grooming or personal care device, a lighted sign, a toy, a scientific instrument, a pump, a transportation device or vehicle, a kitchen device, or a household appliance.

Aspect 70. The article or device of aspect 68, wherein the device includes a light emitting diode.

Aspect 71. A method of culturing cells comprising growing the cells in a medium comprising the extract of aspect 65.

Aspect 72. The method of aspect 71, wherein the cells comprise bacteria or fungi.

Aspect 73. The method of aspect 72, wherein the bacteria or fungi comprise Saccharomyces cerevisiae, a Kluyveromyces species, a Candida species, a Lactobacillus species, Pyrococcus furiosus, Thermus aquaticus, Bacillus stearothermophilus, Thermus filiformis, Thermus thermophiles, a Xanthomonas species, Aspergillus niger or another Aspergillus species, a Trichoderma species, Mucor miehei, a Rhizopus species, a Mortierella species, Clostridium acetobutylicum or another Clostridium species, a Streptomyces species, a Penicillium species, Acetobacter acetii, Bacillus thuringiensis or another Bacillus species, E. coli, a Klebsiella species, or Trichoderma polysporum.

Aspect 74. A plant grown by the process comprising contacting plant gamete cells, a plant reproductive organ, or a plant callus with the extract of aspect 65.

Aspect 75. The plant of aspect 74, wherein the plant is produced by a method comprising the steps of:
(a) contacting the plant callus with the extract;
(b) culturing the plant callus; and
(c) growing the plant from the plant callus.

Aspect 76. A pharmaceutical composition comprising the extract of aspect 65.

Aspect 77. The pharmaceutical composition of aspect 76, further comprising a UV-blocking agent.

Aspect 78. The pharmaceutical composition of aspect 77, wherein the UV-blocking agent is zinc oxide, titanium dioxide, a carotenoid, oxybenzone, octinoxate, homosalate, octisalate, octocrylene, avobenzone, or any combination thereof.

Aspect 79. A sunscreen comprising the extract of aspect 65.

Aspect 80. A paint, ink, dye, or stain comprising the extract of aspect 65.

Aspect 81. A plant coated with the extract of aspect 65.

Aspect 82. An agricultural product coated with the extract of aspect 65.

Aspect 83. The agricultural product of aspect 82, wherein the agricultural product comprises fruits, leaves, seeds, flowers, grains, nuts, stems, vegetables, or mushrooms.

Aspect 84. A method for improving a physiological property of a plant, the method comprising applying the extract of aspect 65 to the plant, wherein the property is improved compared to the property of the same plant that has not been applied the extract.

Aspect 85. The method of aspect 84, wherein the property comprises increased root tension, root length, hormone production, metabolite production, drought tolerance, disease resistance, photosynthesis, carbon dioxide uptake, or any combination thereof.

Aspect 86. The method of aspect 85, wherein the hormone comprises an auxin, abscisic acid, jasmonic acid, salicylic acid, or any combination thereof.

Aspect 87. The method of aspect 85 or 86, wherein the metabolite comprises a phytochrome protein, chlorophyll, lycopene, carotene or another form of vitamin A, vitamin C, or any combination thereof.

Aspect 88. The method of aspect 84, wherein the plant is grass, trees, bushes, shrubs, flower, vines, coffee, soybean, or cotton.

Aspect 89. The method of aspect 88, wherein the grass is growing on a golf course, a lawn, or an athletic playing field.

Aspect 90. The method of aspect 89, wherein the extract is applied in an amount of from about 50 to about 200 mL per square meter of grass.

Aspect 91. The method of aspect 90, wherein about 75 mL of extract are applied per square meter of grass.

Aspect 92. The method of aspect 90, wherein about 150 mL of extract are applied per square meter of grass.

Aspect 93. The method of aspect 90, wherein the extract is applied twice per week.

Aspect 94. The method of aspect 90, wherein the extract is applied in combination with a second compound that promotes plant growth.

Aspect 95. The method of aspect 94, wherein the second compound is applied before the extract.

Aspect 96. The method of aspect 94, wherein the second compound is applied after the extract.

Aspect 97. The method of aspect 94, wherein the second compound is applied simultaneously with the extract.

Aspect 98. The method of aspect 94, wherein the second compound is a polysaccharide, a fertilizer, or a combination thereof.

Aspect 90. The method of aspect 94, wherein the second compound is chitosan, a polyactive carbohydrate, or a combination thereof.

Aspect 100. The method of aspect 99, wherein the second compound is in an aqueous solution having a concentration of from about 0.01% to about 0.05%.

Aspect 101. The method of aspect 100, wherein the second compound has a concentration of about 0.02%.

Aspect 102. The method of aspect 98, wherein the fertilizer is activated sewage sludge.

Aspect 103. An article comprising the extract of aspect 65, wherein the article is coated with the extract, the extract is dispersed throughout the article, or a combination thereof.

Aspect 104. The article of aspect 1.3, wherein the article is made of glass, fiberglass, plastic, metal, wood, fabric, foam, rubber, latex, silicone, or any combination thereof.

Aspect 105. A method of reducing or preventing exposure of an item to UV radiation comprising applying to the item the extract in aspect 65.

Aspect 106. The method of aspect 105, wherein the extract blocks at least 50% of UV radiation from contacting the item.

Aspect 107. The method of aspect 105, wherein the extract blocks at least 50% of longwave UV radiation from contacting the item.

Aspect 108. The method of aspect 105, wherein the extract blocks at least 50% of shortwave UV radiation from contacting the item.

Aspect 109. The method of aspect 105, wherein the item comprises the skin of a subject.

Aspect 110. The method of aspect 105, wherein the item comprises an agricultural product.

Aspect 111. The method of aspect 105, wherein the item comprises a construction material, an aeronautical material, or an aerospace material.

Aspect 112. The method of aspect 105, wherein the item comprises a culture of a microorganism.

Aspect 113. The method of aspect 105, wherein the item comprises a culture of isolated human or animal cells.

Aspect 114. A method for reducing or preventing the growth of barnacles on a surface, the method comprising applying a paint comprising the extract in aspect 65 and chitosan to the surface.

Aspect 115. The method of aspect 114, wherein the chitosan is from 60% to 100% acetylated and has from 3 to 20 glucosamine units, N-acetylglucosamine units, or a combination thereof.

Aspect 116. A cosmetic composition comprising a physiologically acceptable medium and the extract of aspect 65.

Aspect 117. An optical film comprising the extract of aspect 65.

Aspect 118. A method of reducing or preventing exposure of an item to UV radiation comprising applying to the item the extract of aspect 65.

Aspect 119. The method of aspect 118, wherein the extract blocks at least 50% of UV radiation from contacting the item.

Aspect 120. The method of aspect 118, wherein the extract blocks at least approximately 50% of longwave UV radiation from contacting the item.

Aspect 121. The method of aspect 118, wherein the extract blocks at least approximately 50% of shortwave UV radiation from contacting the item.

Aspect 122. The method of aspect 118, wherein the item comprises the skin of a subject.

Aspect 123. The method of aspect 118, wherein the item comprises an agricultural product.

Aspect 124. The method of aspect 118, wherein the item comprises a construction material, and aeronautical, or an aerospace material.

Aspect 125. An article comprising the extract of aspect 65, wherein the article is coated with the extract, the extract is dispersed throughout the article, or a combination thereof.

Aspect 126. The article of aspect 125, wherein the article is made of glass, fiberglass, plastic, metal, wood, fabric, foam, rubber, latex, silicone, or any combination thereof.

Aspect 127. A composition comprising water, the extract of aspect 65, and one or more one or more polysaccharides.

Aspect 128. The composition of aspect 127, wherein the polysaccharide comprises chitosan, a polyactive carbohydrate, or a combination thereof.

Aspect 129. The composition of aspect 127, wherein the extract is less than 1% by weight, less than 0.75% by weight, less than 0.5% by weight, less than 0.25% by weight, less than 0.1% by weight, less than 0.5% by weight, or less than 0.3% by weight of the composition.

Aspect 130. The composition of aspect 127, wherein chitosan is less than 1% by weight, less than 0.75% by weight, less than 0.5% by weight, less than 0.25% by weight, less than 0.1% by weight, less than 0.5% by weight, or less than 0.3% by weight of the composition.

Aspect 131. The composition of aspect 127, wherein the polyactive carbohydrate is less than 1% by weight, less than 0.75% by weight, less than 0.5% by weight, less than 0.25% by weight, less than 0.1% by weight, less than 0.5% by weight, or less than 0.3% by weight of the composition.

Aspect 132. A method for detecting the production of a plant hormone in a plant, the method comprising admixing an extract according to aspect 65 or 66 with a homogenized sample from the plant and detecting a signal.

Aspect 133. The method of aspect 132, wherein the plant hormone comprises auxin, abscisic acid, jasmonic acid, salicylic acid, or any combination thereof.

Aspect 134. The method of aspect 132 or 133, wherein the signal comprises a fluorescence signal.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Preparation of DNA Constructs

The DNA construct was composed of the genetic components described herein and assembled in plasmid vectors (e.g., pYES2, pBAD). Sequences of genes and/or proteins with desired properties were identified in GenBank; these included a gene that encodes HSP70, a gene that encodes RuBisCO large subunit 1, a gene that encodes tonB, a gene that encodes phosphoenol pyruvate carboxykinase, a gene that encodes phosphoenol pyruvate carboxylase, a gene that encodes hydrogenase, and a gene that encodes P-type ATPase. These sequences were synthesized by CloneTex Systems, Inc. (Austin, TX). Other genetic parts were also obtained for inclusion in the DNA constructs including, for example, promoter genes (e.g., GAL1 promoter, araBAD promoter), reporter genes (e.g., enhanced green fluorescent reporter protein), and terminator sequences (e.g., CYC1 terminator, rrnB terminator). These genetic parts included restriction sites for ease of insertion into plasmid vectors.

The cloning of the DNA construct into the biological devices was performed as follows. Sequences of individual genes were amplified by polymerase chain reaction using primers that incorporated restriction sites at their 5′ ends to facilitate construction of the full sequence to be inserted into the plasmid. Genes were then ligated using standard protocols to form an insert. The plasmid was then digested with restriction enzymes according to directions and using reagents provided by the enzymes' supplier (Promega). The complete insert, containing restriction sites on each end, was then ligated into the plasmid. Successful construction of the insert and ligation of the insert into the plasmid were confirmed by gel electrophoresis.

In some experiments, each gene was PCR amplified using gene-specific overlap primers and assembled sequences were sub-cloned into a pYES2 vector. PCR amplified pieces of all fragments were combined using homologous recombination technology (Gibson Assembly). Clones obtained after transformation were sequenced and analyzed for DNA sequence accuracy.

From 5′ to 3′, one version of the construct for making a genelight producing DNA composition or extract includes (a) a gene that encodes HSP70, (b) a gene that encodes RuBisCO large subunit 1, (c) a gene that encodes TonB, (d) a gene that encodes phosphoenol pyruvate carboxykinase, (e) a gene that encodes phosphoenol pyruvate carboxylase, (f) a gene that encodes hydrogenase, (g) a gene that encodes p-type ATPase, and (h) a gene that encodes EGFP (FIGS. 1A-1B).

From 5′ to 3′, a second version of the construct for making a genelight producing DNA composition or extract includes (a) a gene that encodes hydrogenase, (b) a gene that encodes p-type ATPase, (c) a gene that encodes phosphoenol pyruvate carboxylase, (d) a gene that encodes phosphoenol pyruvate kinase, (e) a gene that encodes tonB, (f) a gene that encodes HSP70, (g) a gene that encodes RuBisCO large subunit 1, and (h) a gene that encodes EGFP (FIGS. 2A-2B).

PCR was used to enhance DNA concentration using a Mastercycler Personal 5332 ThermoCycler (Eppendorf North America) with specific sequence primers and the standard method for amplification (Sambrook, J., E. F. Fritsch, and T. Maniatis, 1989, Molecular Cloning: A Laboratory Manual, 2nd ed., Vol. 1, Cold Spring Harbor Laboratory Press: Cold Spring Harbor, NY). Digestion and ligation were used to ensure assembly of DNA synthesized parts using restriction enzymes and reagents (PCR master mix of restriction enzymes: XhoI, KpnI, XbaI, EcoRI, BamHI, and HindIII, with alkaline phosphatase and quick ligation kit, all from Promega).

DNA was quantified using a NanoVue spectrophotometer (GE Life Sciences) and a standard UV/Visible spectrophotometer using the ratio of absorbances at 260 nm and 280 nm. In order to verify final ligations, DNA was visualized and purified via electrophoresis using a Thermo EC-150 power supply.

The DNA construct was made with gene parts fundamental for expression of sequences such as, for example, native and constitutive promoters, reporter genes, and transcriptional terminators or stops. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the method described below.

### Example 2: Selection of Microorganisms and Development of Competent Cells

Some constructs were produced using transfected yeasts (Saccharomyces cerevisiae, ATCC®200892™). Yeast cells were made competent by subjecting them to an electrochemical process adapted from Gietz and Schiestl (Nature Protocols, 2007, 2:35-37). Briefly, a single yeast colony was inoculated into 100 mL YPD (yeast extract peptone dextrose) growth media. Yeast was grown overnight on a shaker at 30° C. to OD₆₀₀=1.0. (Acceptable results were obtained with OD₆₀₀ values ranging from 0.6 to 1.8.) Cells were centrifuged at 2000 rpm in a tabletop centrifuge and resuspended in 10 mL TEL buffer (10 mM Tris-HCl, 1 mM EDTA, 0.1 M LiAc, pH=7.5) and shaken vigorously overnight at room temperature. Alternatively, INVSc1 cells were prepared to be competent using a kit from Sigma-Aldrich, Inc. Cells were again centrifuged and resuspended in 1 mL TEL buffer. Cells prepared in this manner could be stored in the refrigerator for up to one month.

Alternatively, bacterial devices were constructed with one of the following strains of cells: Escherichia coli, ONESHOT® Top10 competent cells from Life Technologies™, BL21 (DE3) E. coli from Novagen, Inc., or DH5α™ E. colifrom Thermo Fisher Scientific.

### Example 3: Transformation of Microbial Cells

Competent cells were stored in the freezer until needed. Cells were thawed on ice and 100 μL of competent cells in TEL buffer were placed in a sterile 1.5 mL microcentrifuge tube. To this was added 5 μL of a 10 mg/mL solution of salmon sperm DNA (carrier DNA). Transforming DNA was added in various amounts. From 1 to 5 μg was sufficient for plasmids from commercial sources, but more DNA was required when transforming yeast with artificial DNA constructs. 10 μL of the DNA device were added to the microcentrifuge tube containing the competent yeast cells and the contents of the tube were mixed. The DNA-yeast suspension was incubated for 30 min at room temperature.

A PLATE solution (consisting of 40% PEG-3350 in 1×TEL buffer) was prepared. 0.7 mL of PLATE solution was added to the DNA-yeast suspension and the contents were mixed thoroughly and incubated for 1 h at room temperature. The mixture was placed in an electromagnetic chamber for 30 minutes. Cells were then heated at 42° C. for 5-10 minutes and 250 μL aliquots were plated on yeast malt agar to which selective growth compounds had been added. Plates were incubated overnight at 30° C.

DNA expression and effectiveness of transformation were determined by fluorescence of the transformed cells expressed in fluorescence units (FSUs) using a 20/20 Luminometer (Promega) according to a protocol provided by the manufacturer. Plasmid DNA extraction, purification, PCR, and gel electrophoresis were also used to confirm transformation. Different transformed devices were obtained. Different types of fluorescent reporter proteins were used (e.g., yellow, red, green, and cyan) for all transformed cells and/or constructs. However, the yellow fluorescent protein was preferred. When no fluorescent reporter protein was assembled, no fluorescence was observed.

S. cerevisiae cells were subjected to transformation with the modified pYES2 plasmids for producing metal- and contaminant-binding components as described above. Transformed yeast cells were incubated for 30 min at 28-30° C. Colonies of transformed yeast cells were selected, their DNA isolated and subjected to PCR amplification. Two control treatments were also carried out: (1) a negative control involving competent yeast and nuclease free water instead of a plasmid and (2) a positive control involving competent yeast with unmodified pYES2 plasmid.

Four clones were selected from a transformed plate and processed for full-length DNA sequencing. A clone with 100% DNA sequence accuracy was selected for further processing and was used to obtain a high concentration of plasmid construct at a mid-scale plasmid purification level. Yeast competent cells were transformed with the recombinant plasmid and selected on synthetic complete (SC) dropout plate deficient in uracil. Well isolated clones were isolated and preserved in YPD medium containing 15% glycerol for storage at −80° C.

### Example 4: Production of Microbial Extracts and Metabolites

### Microbial Extracts Containing Genelight Metabolites

The following non-limiting procedure was used to produce the disclosed extracts:

### Method

(a) Bacteria transformed with the device depicted in FIGS. 1A-1B or yeast transformed with the device FIGS. 2A-2B were fermented at 37° C. for 48 hours, where culture was conducted with 25 mL of device inoculum in 1 L Luria broth and having 1 μg/mL ampicillin and 100 μM isopropyl-β-D-thiogalactopyranoside (IPTG).
(b) The culture was sterilized by autoclaving at 121° C. for 30 minutes and then centrifuged.
(c) The mixture was filtered with an 0.45 μm filter to produce a supernatant composed of the desired extract.

Throughout this application, various publications are referenced. The disclosures of these publications in their entireties are hereby incorporated by reference into this application in order to more fully describe the compounds, compositions, and methods described herein.

### Example 5: Phytochrome Production in Field Grasses

Four grass samples were subjected to four different treatments: (1) treatment with lysate/extract from a biological device including a DNA construct having SEQ ID NO. 10, (2) treatment with lysate/extract from a biological device including a DNA construct having SEQ ID NO. 30, (3) treatment with lysate/extract from non-transformed yeast, and (4) a water control. ELISA testing was performed using strip plates according to manufacturer's directions. The kit used employs a double-antibody sandwich method to determine the concentration of phytochrome in specimens. A microwell plate was coated with purified anti-phytochrome antibody. Experimental samples suspected of containing phytochrome were added to the microwells; this was followed by the addition of horseradish peroxidase (HRP) labeled anti-phytochrome antibody to form an antibody-antigen-enzyme labeled antibody complex. The plate was washed and the substrate tetramethylbenzidine (TMB) was added for color development. HRP catalyzes a reaction that turns TMB blue, then yellow upon the addition of acid. The intensity of the color produced is directly proportional to the concentration of phytochrome in the sample. A microplate reader was used to measure optical density at 450 nm and the phytochrome concentration in the sample was calculated using a standard curve.

Phytochrome levels (in ng/g) for each grass sample are presented in Table 8; six replicates were carried out for each grass sample.

TABLE 8
Amount of Phytochrome in Samples

| Sample | Replicate | Phytochrome (ng/g) |
|---|---|---|
| SEQ ID NO. 10 | 1 | 1191.906 |
| | 2 | 1251.454 |
| | 3 | 1132.899 |
| | 4 | 1247.175 |
| | 5 | 1236.924 |
| | 6 | 1427.685 |
| SEQ ID NO. 30 | 1 | 699.879 |
| | 2 | 696.433 |
| | 3 | 693.102 |
| | 4 | 694.259 |
| | 5 | 685.382 |
| | 6 | 681.851 |
| Non-transformed yeast | 1 | 877.017 |
| | 2 | 876.089 |
| | 3 | 836.094 |
| | 4 | 925.29 |
| | 5 | 827.815 |
| | 6 | 852.371 |
| Water control | 1 | 1312.147 |
| | 2 | 1293.658 |
| | 3 | 1184.195 |
| | 4 | 1256.831 |
| | 5 | 1111.206 |
| | 6 | 1147.337 |

The biological devices having SEQ ID NO. 10 express stronger induction of phytochrome in field grasses compared to other treatments. Inclusion of the phosphoenol pyruvate carboxylase genes in this device may enhance plant photosynthesis by mediating the effects of temperature on photosynthesis.

### Example 6: CO₂ Absorption in Treated Grass

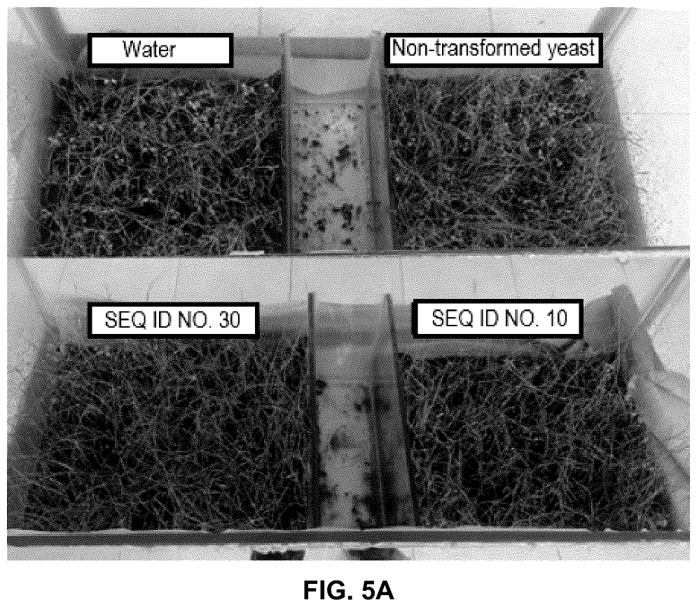

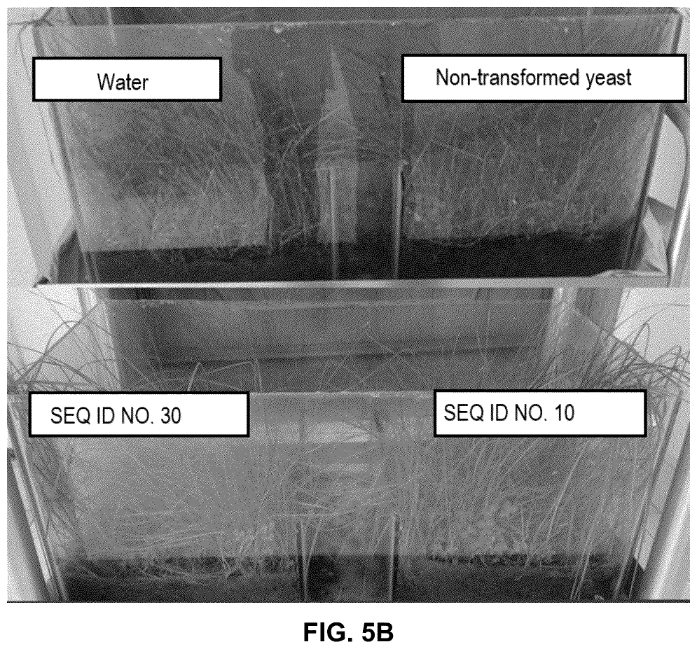

CO₂ measurements were measured using a PASCO wireless CO₂ sensor (model PS-3208). Measurements were collected at 0 hours and collected each day until 20 days' time had elapsed. Treatments with disclosed lysates/extracts were applied at 0, 5, 10, and 15 days. A water control was used, as was a non-transformed yeast control. Experimental conditions included use of a device including a gene that encodes a hydrogenase; a gene that encodes a p-type ATPase; a gene that encodes phosphoenol pyruvate carboxylase; a gene that encodes phosphoenol pyruvate carboxykinase; a gene that encodes tonB; a gene that encodes a heat shock protein; and a gene that encodes RuBisCO large subunit 1 (referred to elsewhere herein as SEQ ID NO. 10 for brevity) and a device including each of the above except for phosphoenol pyruvate carboxylase and phosphoenol pyruvate carboxykinase (referred to elsewhere herein as SEQ ID NO. 30 for brevity). Grass at 0 days is shown in FIG. 5A and grass at 20 days is shown in FIG. 5B.

Carbon dioxide ppm over time are shown in Table 9 with treatments using a disclosed device/composition occurring at 0, 5, 10, and 15 days:

TABLE 9
CO₂ ppm for Grass Samples

| | | | Non- | | | |
|---|---|---|---|---|---|---|
| | | | transformed | SEQ ID | SEQ ID | Temperature |
| Time | | Water | Yeast | NO. 30 | NO. 10 | (° F.) |
| 0 | Hours | 400 | 400 | 400 | 408 | 60.8 |
| Day | 1 | 464 | 402 | 393 | 400 | 62.6 |
| Day | 2 | 449 | 399 | 388 | 393 | 64.4 |
| Day | 3 | 451 | 411 | 400 | 399 | 64.4 |
| Day | 4 | 420 | 430 | 401 | 402 | 60.8 |
| Day | 5 | 495 | 457 | 384 | 377 | 62.6 |
| Day | 6 | 517 | 503 | 372 | 370 | 64.4 |
| Day | 7 | 587 | 426 | 355 | 369 | 60.8 |
| Day | 8 | 522 | 500 | 329 | 322 | 68 |
| Day | 9 | 480 | 477 | 305 | 341 | 69.8 |
| Day | 10 | 511 | 465 | 323 | 344 | 57.2 |
| Day | 11 | 533 | 502 | 289 | 292 | 68 |
| Day | 12 | 544 | 518 | 291 | 288 | 68 |
| Day | 13 | 528 | 517 | 334 | 287 | 68 |
| Day | 14 | 477 | 519 | 300 | 277 | 60.8 |
| Day | 15 | 602 | 550 | 350 | 348 | 59.9 |
| Day | 16 | 607 | 584 | 295 | 274 | 59 |
| Day | 17 | 785 | 706 | 263 | 270 | 64.4 |
| Day | 18 | 952 | 799 | 285 | 206 | 59 |
| Day | 19 | 881 | 803 | 255 | 276 | 59.9 |
| Day | 20 | 885 | 642 | 261 | 229 | 60.8 |

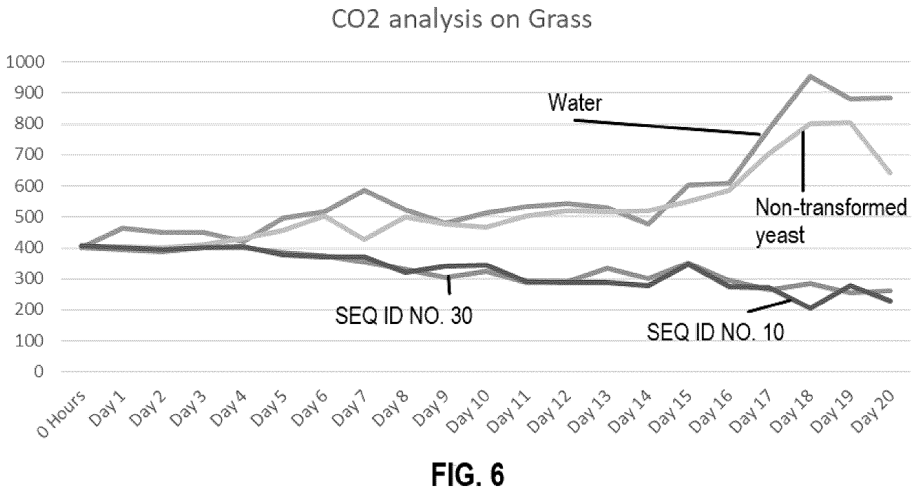

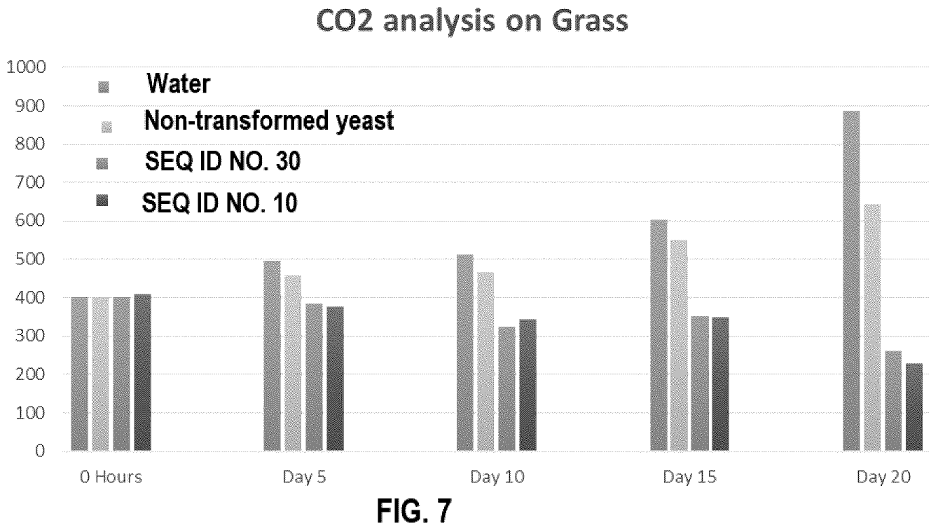

FIGS. 6-7 show graphical representations of CO₂ over time. Clearly, grass treated with a SEQ ID NO. 10 extract/lysate shows a marked uptake (reduction) of CO₂ in the environment where field grass was grown, followed by a SEQ ID NO. 30 extract/lysate, at different stages of the time-course experiment during 3 weeks, as compared to control. These results corroborate the efficacy of a SEQ ID NO. 10 extract/lysate, which produces the phosphoenol pyruvate carboxylase protein and a phosphoenol pyruvate carboxykinase protein in addition to the other proteins that are also present in a SEQ ID NO. 30 extract/lysate.

### Example 7: Enhancement of Tomato Growth

Extracts were produced for the treatment of plants as follows. Non-transformed yeasts or yeasts producing a polyactive carbohydrate, yeasts containing a DNA construct having SEQ ID NO. 30, and yeasts containing a DNA construct having SEQ ID NO. 10 were fermented in yeast malt medium with 1 mg/mL of glucosamine, 2% (w/v) raffinose, and 1% (w/v) galactose at 30° C. for 72 hours. The resultant solutions were autoclaved for 15 minutes, centrifuged at 9000 rpm for 10 minutes, and filtered using a 0.45 μm filter.

Cherry tomato seeds were treated with the lysates/extracts produced above prior to sowing. A water-based control was also used. Once the seeds sprouted, the plants were treated twice per week with the same treatment applied to the seeds. One month after sowing, the plants were transplanted to soil. Once in soil, the plants were treated two times per month. For the first harvest, the plants had experienced 6 total treatments, and for the second harvest, the plants had experienced 9 total treatments. Results are shown in Table 10 below:

TABLE 10
Tomato Plant Results

| | Weight | Total | |
|---|---|---|---|
| | per | harvest | Number |
| | tomato | weight | of |
| | (g) | (g) | tomatoes |
| Control with water 1^{st} Harvest | 23.2 ± 4.8 | 394.6 | 17 |
| Control with water 2^{nd} Harvest | 20.3 ± 5.3 | 487.3 | 24 |
| Non-transformed Yeast 1^{st} Harvest | 24.3 ± 2.6 | 413.3 | 17 |
| Non-transformed Yeast 2^{nd} Harvest | 19.6 ± 5.3 | 411.4 | 21 |
| Polyactive carbohydrate 1^{st} Harvest | 21.8 ± 3.9 | 500.3 | 23 |
| Polyactive carbohydrate 2^{nd} Harvest | 20.3 ± 5 | 489.6 | 24 |
| SEQ ID NO. 30 1^{st} Harvest | 24.1 ± 6.4 | 625.6 | 26 |
| SEQ ID NO. 30 2^{nd} Harvest | 22.2 ± 5.7 | 664.6 | 30 |
| SEQ ID NO. 10 1^{st} Harvest | 23.3 ± 2.6 | 559.2 | 24 |
| SEQ ID NO. 10 2^{nd} Harvest | 23.5 ± 5.9 | 869.4 | 37 |

Tomato plants treated with the SEQ ID NO. 10 lysate/extract show markedly higher harvesting amount and weight per each tomato fruit, especially at the second harvest, and those treated with SEQ ID NO. 30 follow, but both SEQ ID NO. 10 and SEQ ID NO. 30 show higher total harvest amount than the water control, the non-transformed yeast control, and the polyactive carbohydrate treatment, although they all show similar amount of weight per tomato at the first harvest. It is believed that application of SEQ ID NO. 10-containing lysates/extracts stimulate plant hormones related to production of fruits, such as tomato. These results further demonstrate an increasing effect of the SEQ ID NO. 10 treatment along with time, especially after 3 months of growth and treatment.

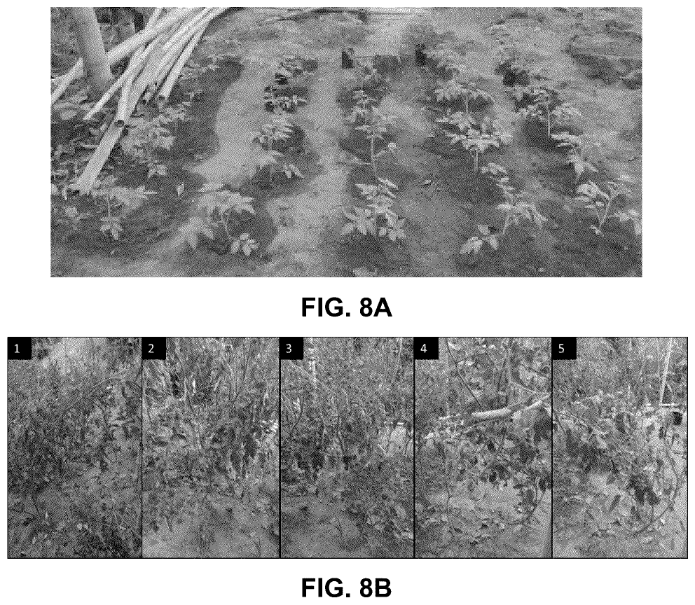

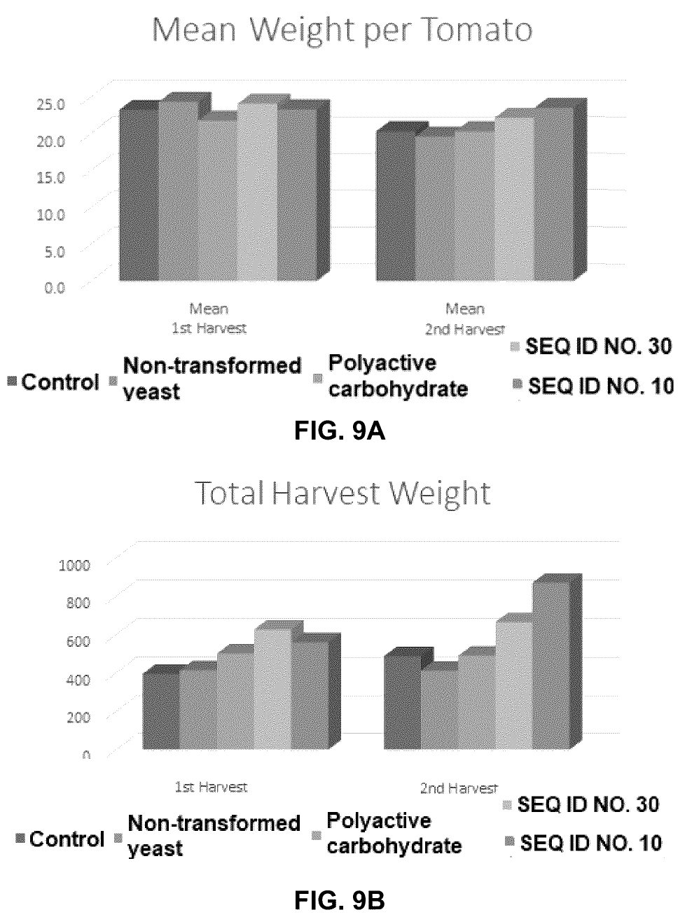

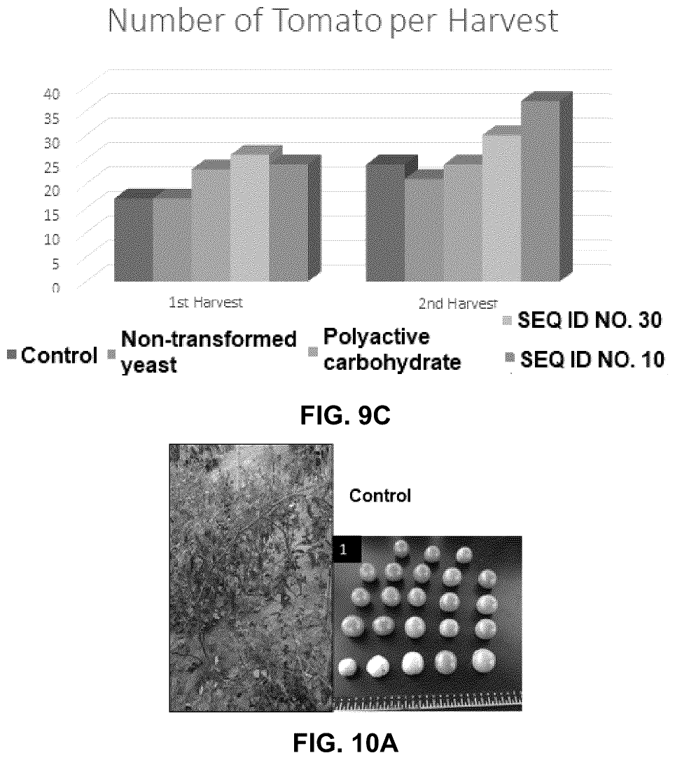

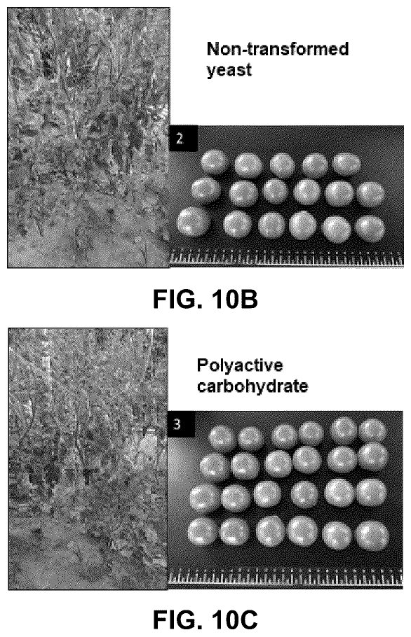

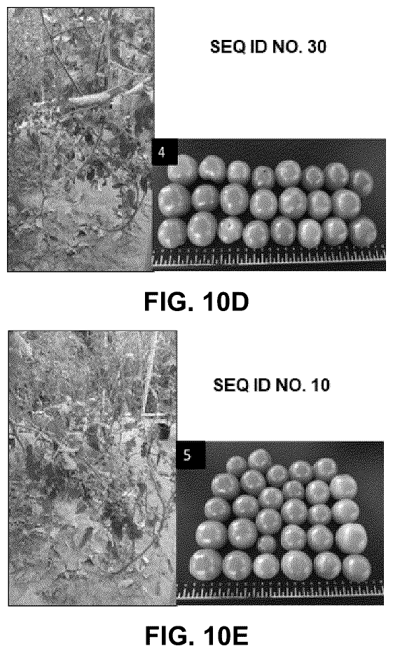

Furthermore, effects of SEQ ID NO. 10 treatment on growth of the tomato are clearly confirmed by the marked reddish color of the tomato fruits as compared to other treatments as seen in FIGS. 10A-10E. This is likely an expression of the stronger induction of carotene/lycopene and phytochrome. SEQ ID NO. 10 also shows a stronger positive effect on tomato plant growth by inducing more foliage and greener color to field tomato leaves (see FIGS. 8A-8B, 10A-10E).

### Example 8: Germination of Corn Seeds and Protein Production Therefrom

Yeast samples (controls as well as yeasts containing SEQ ID NO. 10 or SEQ ID NO. 30) were grown in yeast malt broth media and cultures were induced with 1% raffinose and 0.5% galactose. Fermentation was conducted at 30° C. Culture samples were taken at 0, 24, 28, 72, and 96 hours and evaluated for optical density, pH, redox capability, and colony count. Samples were sonicated to lyse the yeast cells, centrifuged, and filtered through a 0.45 μm filter. Filtrates were retained for protein concentration measurements and as treatments to induce corn germination.

To obtain colony count, sample aliquots were serially diluted to a 10⁻² dilution. Dilution aliquots were mixed with 100 μL Trypan blue stain. 10 μL of this mixture was used to fill in each side of a disposable Neubauer improved hemocytometer and placed under a microscope to count the cells in each of eight squares. After recording the number of cells in each square, the concentration of cells per mL was calculated using the mean cell count from the 8 measurements.

To determine protein concentration, each aliquot was subjected to the Bradford Protein Assay on a Perkin-Elmer UV/vis spectrophotometer. Each aliquot was used as is for one measurement but was also purified as follows: a 10 kDa molecular weight cutoff centrifuge device included 3 mL of extract mixed with 17 mL of PBS, then again missed with 20 mL of deionized water to rinse and remove the PBS from the protein on the filter. Dilutions using 100 μL and 60 μL of each extract were mixed with water to achieve 600 μL total of sample, to which 2.4 mL of Bradford reagent (Coomassie blue, phosphoric acid, ethanol, and water) were added. The samples were incubated at 27° C. for from 5 to 15 min. Absorption was measured at 595 nm. Protein concentrations were then calculated and compared to individual absorptions of proteins produced by the disclosed devices.

Corn seeds were submerged in each extract for 45 minutes and then placed in sterile wet seed germinating paper inside a plastic container. 10 corn seeds were used per treatment. These included extracts from yeasts containing SEQ ID NO. 10 or SEQ ID NO. 30 sampled at 0, 24, 48, 72, or 96 h, as well as a non-transformed yeast extract and a water control.

Seeds were observed for germination at 24, 36, 48, and 72 hours. Seeds were sprayed with deionized water every day to keep the environment humid. Early germination % was calculated after observing the seeds at 24 h, and length of roots was measured for each germinated seed at the end of the experiment, 72 hours after the treatment.

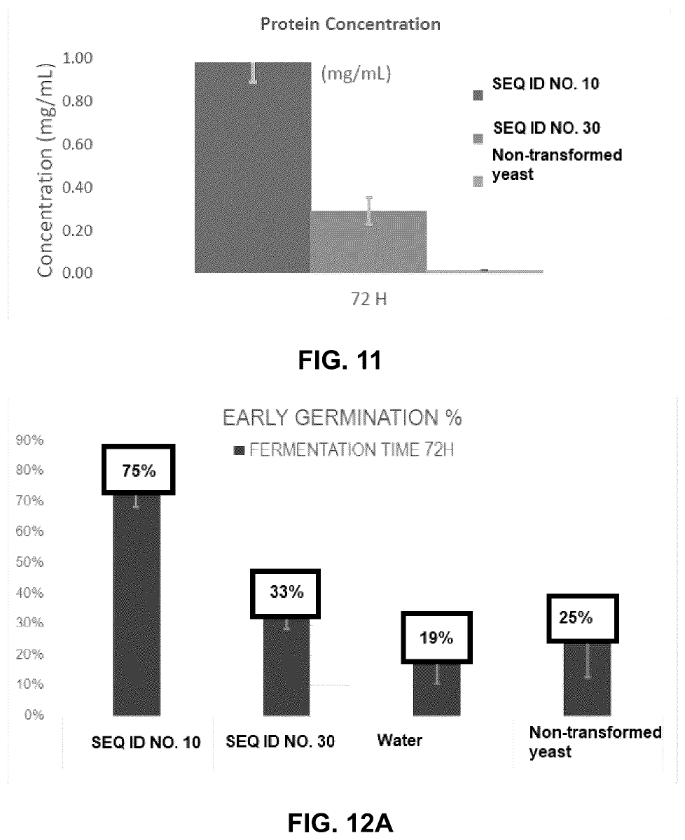

Protein concentration measurements are presented in Table 11 below and FIG. 11:

TABLE 11
Protein Concentration in Yeast Cultures

| | Yeast | Yeast | |
|---|---|---|---|
| | transformed | transformed | Non- |
| | with SEQ | with SEQ | transformed |
| | ID NO. 10 | ID NO. 30 | yeast |
| | 0.91 | 0.27 | 0.01 |
| | 0.61 | 0.23 | 0.012 |
| | 1.08 | 0.38 | 0.005 |
| | 0.73 | 0.20 | 0.018 |
| | 0.96 | 0.25 | 0.026 |
| Mean | 0.86 | 0.27 | 0.014 |
| Standard | 0.19 | 0.07 | 0.0081 |
| Deviation | | | |

Colon count measurements are presented in Table 12 below:

TABLE 12
Yeast Colony Count

| | Yeast | Yeast | | |
|---|---|---|---|---|
| | transformed | transformed | Non- | |
| | with SEQ | with SEQ | transformed | Water |
| | ID NO. 10 | ID NO. 30 | yeast | control |
| | 1.74 × 10⁸ | 1.26 × 10⁸ | 3.5 × 10⁷ | 0 |
| | 1.34 × 10⁸ | 1.22 × 10⁸ | 4.0 × 10⁷ | 0 |
| | 1.26 × 10⁸ | 1.36 × 10⁸ | 4.0 × 10⁷ | 0 |
| | 1.52 × 10⁸ | 1.18 × 10⁸ | 4.75 × 10⁷ | 0 |
| | 1.54 × 10⁸ | 9 × 10⁷ | 4.6 × 10⁷ | 0 |
| | 1.76 × 10⁸ | 1 × 10⁸ | 4.0 × 10⁷ | 0 |
| | 1.38 × 10⁸ | 1 × 10⁸ | 4.7 × 10⁷ | 0 |
| | 1.60 × 10⁸ | 9.4 × 10⁷ | 4.6 × 10⁷ | 0 |
| Mean | 1.52 × 10⁸ | 1.11 × 10⁸ | 4.27 × 10⁷ | 0 |
| Standard | 1.82 × 10⁷ | 1.69 × 10⁷ | 4.6 × 10⁶ | 0 |
| deviation | | | | |

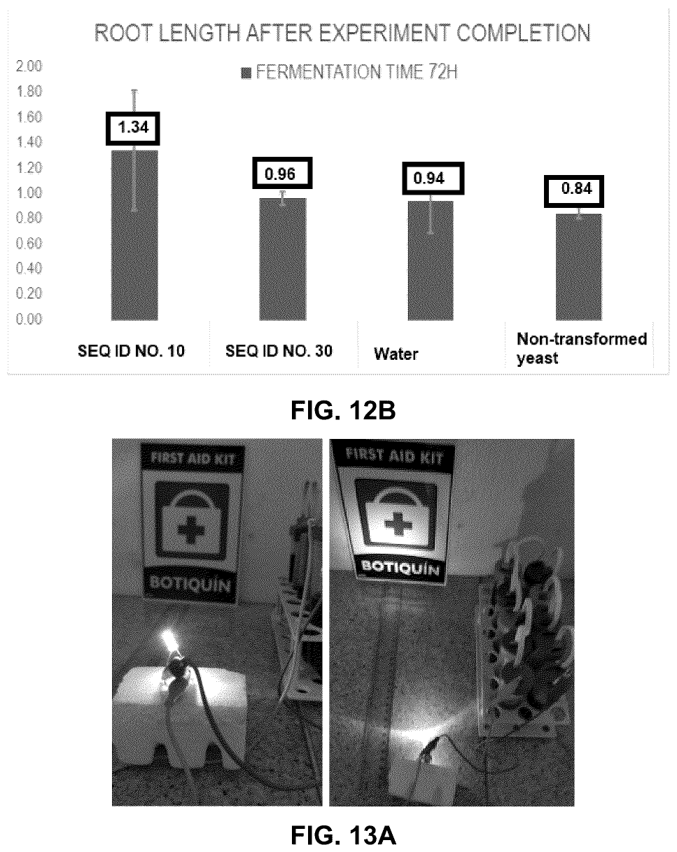

Early germination and root length results are presented in FIGS. 12A-12B.

### Example 9: Bio-Battery Powering LEDs

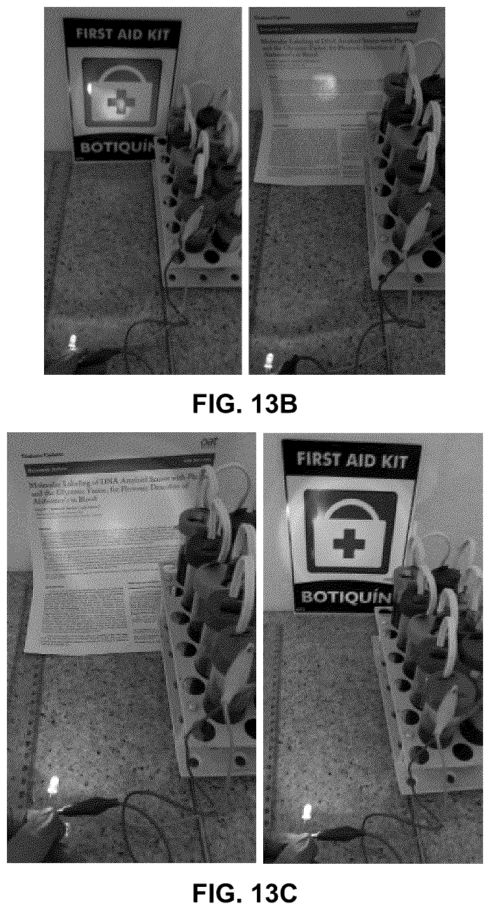

Lysates and extracts were produced using an E. coli culture transformed with a vector having SEQ ID NO. 9. The extract could be used to power different colors of LEDs (see FIGS. 13A-13C). In some cases the extract was mixed in a 2:1 ratio with an extract of a culture Acidithiobacillus ferrooxidans. An exemplary mixture was used to power a white LED and, on the first day of measurement, luminescence was 181 lux, 2.61×10¹⁵ photons were produced, voltage in the circuit was 4.1 V, and current in the circuit was 331 ρA.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be exemplary.

What is claimed is:

1. A DNA construct comprising the following genetic components:
(a) a gene that encodes a hydrogenase;
(b) a gene that encodes a p-type ATPase;
(c) a gene that encodes phosphoenol pyruvate carboxylase;
(d) a gene that encodes phosphoenol pyruvate carboxykinase;
(e) a gene that encodes tonB;
(f) a gene that encodes a heat shock protein; and
(g) a gene that encodes RuBisCO large subunit 1.

2. The DNA construct of claim 1, wherein the gene that encodes the hydrogenase has SEQ ID NO. 6 or at least 70% homology thereto.

3. The DNA construct of claim 1, wherein the gene that encodes the p-type ATPase has SEQ ID NO. 7 or at least 70% homology thereto.

4. The DNA construct of claim 1, wherein the gene that encodes phosphoenol pyruvate carboxylase has SEQ ID NO. 5 or at least 70% homology thereto.

5. The DNA construct of claim 1, wherein the gene that encodes phosphoenol pyruvate carboxykinase has SEQ ID NO. 4 or at least 70% homology thereto.

6. The DNA construct of claim 1, wherein the gene that encodes the tonB has SEQ ID NO. 3 or at least 70% homology thereto.

7. The DNA construct of claim 1, wherein the gene that encodes the heat shock protein has SEQ ID NO. 1 or at least 70% homology thereto.

8. The DNA construct of claim 1, wherein the gene that encodes the RuBisCO large subunit 1 has SEQ ID NO. 2 or at least 70% homology thereto.

9. The DNA construct of claim 1, wherein the construct further comprises at least one promoter.

10. The DNA construct of claim 9, wherein the at least one promoter is a T3 promoter, a T7 promoter, an iron promoter, a GAL1 promoter, or any combination thereof.

11. The DNA construct of claim 10, wherein the at least one promoter is a GAL1 promoter, and the GAL1 promoter is positioned before the gene that encodes the hydrogenase; the gene that encodes the p-type ATPase, the gene that encodes phosphoenol pyruvate carboxylase, the gene that encodes phosphoenol pyruvate carboxykinase; the gene that encodes tonB; the gene that encodes the heat shock protein; the gene that encodes RuBisCO large subunit 1, or any combination thereof.

12. The DNA construct of claim 1, wherein the DNA construct further comprises a fluorescent reporter protein.

13. The DNA construct of claim 12, wherein the fluorescent reporter protein is a green fluorescent protein having SEQ ID NO. 8 or at least 70% homology thereto.

14. The DNA construct of claim 1, wherein the construct comprises from 5′ to 3′ the following genetic components in the following order: (a) a gene that encodes a hydrogenase; (b) a gene that encodes a p-type ATPase; (c) a gene that encodes phosphoenol pyruvate carboxylase; (d) a gene that encodes phosphoenol pyruvate carboxykinase; (e) a gene that encodes tonB; (f) a gene that encodes a heat shock protein; and (g) a gene that encodes RuBisCO large subunit 1.

15. The DNA construct of claim 1, wherein the construct comprises from 5′ to 3′ the following genetic components in the following order: (a) a gene that encodes a hydrogenase having SEQ ID NO. 6 or at least 70% homology thereto; (b) a gene that encodes a p-type ATPase having SEQ ID NO. 7 or at least 70% homology thereto; (c) a gene that encodes phosphoenol pyruvate carboxylase having SEQ ID NO. 5 or at least 70% homology thereto; (d) a gene that encodes phosphoenol pyruvate carboxykinase having SEQ ID NO. 4 or at least 70% homology thereto; (e) a gene that encodes tonB having SEQ ID NO. 3 or at least 70% homology thereto; (f) a gene that encodes a heat shock protein having SEQ ID NO. 1 or at least 70% homology thereto; and (g) a gene that encodes RuBisCO large subunit 1 having SEQ ID NO. 2 or at least 70% homology thereto.

16. The DNA construct of claim 1, wherein the construct comprises from 5′ to 3′ the following genetic components in the following order: (a) a gene that encodes a hydrogenase; (b) a CYC1 terminator; (c) a GAL1 promoter; (d) a gene that encodes a p-type ATPase; (e) a CYC1 terminator; (f) a GAL1 promoter; (g) a gene that encodes phosphoenol pyruvate carboxylase; (h) a CYC1 terminator; (i) a GAL1 promoter; (j) a gene that encodes phosphoenol pyruvate carboxykinase; (k) a CYC1 terminator; (l) a GAL1 promoter; (m) a gene that encodes tonB; (n) a CYC1 terminator; (o) a GAL1 promoter; (p) a gene that encodes a heat shock protein; (q) a CYC1 terminator; (r) a GAL1 promoter; and (s) a gene that encodes RuBisCO large subunit 1.

17. The DNA construct of claim 1, wherein the construct comprises from 5′ to 3′ the following genetic components in the following order: (a) a gene that encodes a hydrogenase having SEQ ID NO. 6 or at least 70% homology thereto; (b) a CYC1 terminator; (c) a GAL1 promoter; (d) a gene that encodes a p-type ATPase having SEQ ID NO. 7 or at least 70% homology thereto; (e) a CYC1 terminator; (f) a GAL1 promoter; (g) a gene that encodes phosphoenol pyruvate carboxylase having SEQ ID NO. 5 or at least 70% homology thereto; (h) a CYC1 terminator; (i) a GAL1 promoter; (j) a gene that encodes phosphoenol pyruvate carboxykinase having SEQ ID NO. 4 or at least 70% homology thereto; (k) a CYC1 terminator; (l) a GAL1 promoter; (m) a gene that encodes tonB having SEQ ID NO. 3 or at least 70% homology thereto; (n) a CYC1 terminator; (o) a GAL1 promoter; (p) a gene that encodes a heat shock protein having SEQ ID NO. 1 or at least 70% homology thereto; (q) a CYC1 terminator; (r) a GAL1 promoter; and (s) a gene that encodes RuBisCO large subunit 1 having SEQ ID NO. 2 or at least 70% homology thereto.

18. The DNA construct of claim 1, wherein the DNA construct has SEQ ID NO. 10.

19. A vector comprising the DNA construct of claim 1.

20. The vector of claim 19, wherein the vector is a plasmid.

21. The vector of claim 20 wherein the plasmid is pWLneo, pSV2cat, pOG44, pXT1, pSG, pSVK3, pBSK, pBSKII, pYES, pYES2, pET, pBAD, pUC, or pUC19.

22. The vector of claim 21, wherein the plasmid is pYES2.

23. A biological device comprising host cells transformed with the DNA construct of claim 1.

24. The biological device of claim 23, wherein the host cells comprise fungi or bacteria.

25. The biological device of claim 24, wherein the fungi comprise Saccharomyces cerevisiae.

26. An extract produced by culturing the biological device of claim 23 in a culture medium.

27. A battery comprising an electrolyte, wherein the electrolyte comprises the extract of claim 26.

28. An article or device comprising the extract of claim 26.

29. The article or device of claim 28, wherein the article or device comprises a standalone light, an indicator light on a larger device, a lighted panel on a larger device, a computer, a tablet, a smartphone, a medical device, a portable heating device, a portable cooling device, a grooming or personal care device, a lighted sign, a toy, a scientific instrument, a pump, a transportation device or vehicle, a kitchen device, or a household appliance.

30. The article or device of claim 28, wherein the device includes a light emitting diode.


SEQUENCE LISTING (30 sequences)

SEQ ID NO: 1 - DNA, 1854 bp, synthetic construct
  source 1..1854: mol_type = other DNA
ggtaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaactcgag

SEQ ID NO: 2 - DNA, 1467 bp, synthetic construct
  source 1..1467: mol_type = other DNA
atgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaa

SEQ ID NO: 3 - DNA, 735 bp, synthetic construct
  source 1..735: mol_type = other DNA
atgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaa

SEQ ID NO: 4 - DNA, 1869 bp, synthetic construct
  source 1..1869: mol_type = other DNA
atgccaccacagctgcaaaacggtctgaacctgagcgctaaagtggttcagggcagcctggattccctgccacaggctgttcgtgagttcctggagaacaacgcagagctgtgccaaccggatcacattcacatctgcgatggttccgaagaggagaacggtcgtctgctgggtcagatggaggaagaaggcatcctgcgtcgcctgaagaagtacgataactgttggctggctctgaccgatccacgtgatgtggctcgcatcgagtccaagactgttatcgttacccaagagcaacgcgatactgtgccgatcccgaagactggcctgagccagctgggtcgctggatgtctgaagaggactttgagaaagcgttcaacgctcgctttccaggttgcatgaaaggtcgtaccatgtacgtgatcccgttcagcatgggtccactgggttctccactgtctaagatcggcattgagctgactgactctccatacgttgttgcttctatgcgtatcatgacccgtatgggcactccagttctggaagcggttggtgatggcgagtttgtgaaatgtctgcactccgtgggctgtccactgccactgcagaaaccactggttaacaactggccatgcaacccagagctgaccctgatcgctcacctgccagatcgtcgtgaaatcatctcctttggttctggttatggtggtaactctctgctgggcaagaaatgcttcgctctgcgtatggcgagccgtctggcaaaggaagagggctggctggctgaacacatgctgattctgggcattaccaatccggagggtgagaagaagtacctggcagctgccttcccgtccgcttgtggtaagaccaatctggccatgatgaacccgtccctgccaggctggaaggtagagtgcgttggcgacgacattgcctggatgaagttcgatgcgcaaggccatctgcgtgccatcaatccggagaacggtttctttggtgttgctccaggtacttccgtaaagactaacccgaatgccatcaagaccatccagaagaacaccatcttcaccaacgtagcggaaacttccgacggtggcgtttactgggagggcattgatgaaccactggccagcggtgttactatcacctcctggaagaacaaagagtggagcagcgaagatggcgagccatgtgcccacccgaactctcgcttctgcactccagcctctcagtgtccaatcattgacgctgcgtgggagtctccagaaggcgttccgattgagggtatcatcttcggtggtcgtcgtccagcaggtgtgccactggtttacgaagctctgtcttggcagcacggtgtattcgttggtgctgcaatgcgctctgaggccaccgctgctgctgaacacaaaggcaagatcatcatgcacgatccgtttgcgatgcgtccgttcttcggttacaacttcggcaagtacctggctcactggctgagcatggctcagcatccagctgcgaaactgccgaagatttttcacgtaaactggtttcgtaaagacaaagaaggcaagttcctgtggccaggtttcggtgagaacagccgtgttctggagtggatgttcaatcgtatcgacggtaaagcatccaccaaactgactccgatcggctacatcccgaaggaggacgcactgaacctgaaaggtctgggtcatatcaacatgatggagctgttcagcatcagcaaagagttctgggagaaagaggttgaggacatcgagaagtacctggaggaccaggtgaacgcagacctgccgtgcgaaatcgaacgtgagatcctggcactgaaacagcgtatctctcaaatgtaa

SEQ ID NO: 5 - DNA, 2904 bp, synthetic construct
  source 1..2904: mol_type = other DNA
atggcaaaccgtaagctggagaagatggcatctatcgacgttcacctgcgtcagctggttccaggtaaggtaagcgaggacgacaaactggttgaatacgacgctctgctgctggatcgtttcctggacattctgcaagacctgcacggtgaagacctgcgtgagactgtacaagagctgtacgaacattctgccgaatacgagggtaagcacgaaccgaagaaactggaagagctgggtagcgtgctgacctctctggacccaggtgattccattgttatcgctaaagcattcagccacatgctgaacctggctaacctggctgaagaagtgcagatcgcctaccgtcgtcgtatcaagaagctgaagaaaggtgacttcgttgatgagtcctctgctaccaccgagagcgacctggaggagactttcaagaaactggttggcgacctgaacaaatctccagaagaaatcttcgacgctctgaagaaccagactgtggatctggtactgactgcgcatccgacccaaagcgtacgtcgcagcctgctgcagaaacacggtcgtatccgtgactgcctggctcagctgtacgcgaaagacatcactccagatgacaaacaagaactggatgaagccctgcaacgtgaaatccaagcagcatttcgcaccgacgagatcaaacgtactccaccgactccacaggacgaaatgcgtgcaggtatgtcttacttccacgagactatctggaaaggtgtgccgaagttcctgcgtcgtgttgataccgctctgaagaacatcggtatcgaagaacgcgtgccgtacaacgctccgctgattcagtttagctcctggatgggtggtgatcgtgatggcaatccacgcgtaactccagaggtgactcgtgacgtgtgtctgctggctcgtatgatggctgcaaccatgtacttcaaccagattgaggacctgatgttcgagatgtccatgtggcgttgcaacgacgagctgcgtgcacgtgcagatgaagtacacgctaactctcgtaaagatgctgctaaacactacatcgagttctggaaatccatcccgaccaccgagccgtaccgtgttatcctgggtgacgttcgtgacaaactgtatcatacccgtgaacgtgcacaccaactgctgtctaacggtcacagcgatgtaccagttgaagcgaccttcatcaacctggagcaattcctggaaccgctggaactgtgctaccgttccctgtgttcttgtggtgatcgtccgatcgcagacggttccctgctggacttcctgcgtcaagtgtccaccttcggtctgagcctggttcgcctggacatccgtcaggagagcgaccgtcacactgacgtgctggacgccatcactacccacctggacatcggctcctatcgtgaatggtctgaagaacgtcgccaggagtggctgctgtctgaactgtctggtaaacgtccgctgttcggctccgacctgccgaagaccgaggaaatcgctgatgtgctggataccttccacgtgatcgcagaactgccagcagacagctttggtgcatacatcatcagcatggctactgctccatccgatgttctggctgttgaactgctgcaacgtgaatgccgtgtgaagcaaccgctgcgtgttgtaccactgtttgagaaactggcagacctggaggctgctccagctgccgttgctcgtctgttcagcgttgattggtacaagaatcgtatcaatggtaaacaggaggttatgatcggctattccgacagcggtaaagacgctggtcgtctgtctgctgcttggcagctgtacaaagcgcaggaagaactggttaaggtggcgaaagaatacggtgttaagctgaccatgtttcatggtcgtggtggtactgtaggtcgtggtggtggtccaacccacctggcgatcctgtctcaaccaccggataccatcaacggctctctgcgtgttaccgttcagggtgaagttatcgaacagagcttcggtgaggagcacctgtgcttccgtaccctgcaacgcttcaccgcagcaactctggaacacggtatgcgtccaccaatctctccgaaaccggaatggcgtgcactgctggatgagatggctgtggttgctaccgaagaataccgtagcgtagtgttccaggaaccacgtttcgttgaatactttcgcctggccactccagagctggaatacggtcgtatgaacattggctctcgtccaagcaaacgcaaaccgtccggtggcatcgaatccctgcgtgcaatcccgtggatcttcgcgtggactcagactcgcttccacctgccagtgtggctgggtttcggttccgctattcgtcacgtgatcgagaaagacgttcgcaacctgcacatgctgcaagacatgtaccaacactggccattcttccgtgttactatcgacctgatcgaaatggtgttcgctaaaggtgatccaggtattgcagcgctgtacgataagctgctggtgtctgaagagctgtggccattcggtgagaaactgcgtgctaacttcgaagagactaagaagctgatcctgcaaaccgctggccacaaagacctgctggaaggtgatccatatctgaaacaacgtctgcgtctgcgtgactcttacatcaccaccctgaatgtatgtcaggcgtacaccctgaaacgtatccgtgacccgtcctaccacgttaccctgcgtccgcatatctccaaagaaatcgctgagagcagcaaaccagctaaggagctgatcgaactgaacccgacctctgagtacgcaccaggcctggaggacactctgattctgaccatgaaaggcattgcagcaggtctgcagaacactggttaa

SEQ ID NO: 6 - DNA, 1557 bp, synthetic construct
  source 1..1557: mol_type = other DNA
atgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgattta

SEQ ID NO: 7 - DNA, 2199 bp, synthetic construct
  source 1..2199: mol_type = other DNA
atgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataa

SEQ ID NO: 8 - DNA, 717 bp, synthetic construct
  source 1..717: mol_type = other DNA
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 9 - DNA, 18517 bp, synthetic construct
  source 1..18517: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggcgcgcctttgtttaactttaagaaggagagctcaatatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaatttgtttaactttaagaaggagatgctagcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaacatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccattttgtttaactttaagaaggagatgaattcatgccaccacagctgcaaaacggtctgaacctgagcgctaaagtggttcagggcagcctggattccctgccacaggctgttcgtgagttcctggagaacaacgcagagctgtgccaaccggatcacattcacatctgcgatggttccgaagaggagaacggtcgtctgctgggtcagatggaggaagaaggcatcctgcgtcgcctgaagaagtacgataactgttggctggctctgaccgatccacgtgatgtggctcgcatcgagtccaagactgttatcgttacccaagagcaacgcgatactgtgccgatcccgaagactggcctgagccagctgggtcgctggatgtctgaagaggactttgagaaagcgttcaacgctcgctttccaggttgcatgaaaggtcgtaccatgtacgtgatcccgttcagcatgggtccactgggttctccactgtctaagatcggcattgagctgactgactctccatacgttgttgcttctatgcgtatcatgacccgtatgggcactccagttctggaagcggttggtgatggcgagtttgtgaaatgtctgcactccgtgggctgtccactgccactgcagaaaccactggttaacaactggccatgcaacccagagctgaccctgatcgctcacctgccagatcgtcgtgaaatcatctcctttggttctggttatggtggtaactctctgctgggcaagaaatgcttcgctctgcgtatggcgagccgtctggcaaaggaagagggctggctggctgaacacatgctgattctgggcattaccaatccggagggtgagaagaagtacctggcagctgccttcccgtccgcttgtggtaagaccaatctggccatgatgaacccgtccctgccaggctggaaggtagagtgcgttggcgacgacattgcctggatgaagttcgatgcgcaaggccatctgcgtgccatcaatccggagaacggtttctttggtgttgctccaggtacttccgtaaagactaacccgaatgccatcaagaccatccagaagaacaccatcttcaccaacgtagcggaaacttccgacggtggcgtttactgggagggcattgatgaaccactggccagcggtgttactatcacctcctggaagaacaaagagtggagcagcgaagatggcgagccatgtgcccacccgaactctcgcttctgcactccagcctctcagtgtccaatcattgacgctgcgtgggagtctccagaaggcgttccgattgagggtatcatcttcggtggtcgtcgtccagcaggtgtgccactggtttacgaagctctgtcttggcagcacggtgtattcgttggtgctgcaatgcgctctgaggccaccgctgctgctgaacacaaaggcaagatcatcatgcacgatccgtttgcgatgcgtccgttcttcggttacaacttcggcaagtacctggctcactggctgagcatggctcagcatccagctgcgaaactgccgaagatttttcacgtaaactggtttcgtaaagacaaagaaggcaagttcctgtggccaggtttcggtgagaacagccgtgttctggagtggatgttcaatcgtatcgacggtaaagcatccaccaaactgactccgatcggctacatcccgaaggaggacgcactgaacctgaaaggtctgggtcatatcaacatgatggagctgttcagcatcagcaaagagttctgggagaaagaggttgaggacatcgagaagtacctggaggaccaggtgaacgcagacctgccgtgcgaaatcgaacgtgagatcctggcactgaaacagcgtatctctcaaatgtaacccgggtttgtttaactttaagaaggagatgatatccatggcaaaccgtaagctggagaagatggcatctatcgacgttcacctgcgtcagctggttccaggtaaggtaagcgaggacgacaaactggttgaatacgacgctctgctgctggatcgtttcctggacattctgcaagacctgcacggtgaagacctgcgtgagactgtacaagagctgtacgaacattctgccgaatacgagggtaagcacgaaccgaagaaactggaagagctgggtagcgtgctgacctctctggacccaggtgattccattgttatcgctaaagcattcagccacatgctgaacctggctaacctggctgaagaagtgcagatcgcctaccgtcgtcgtatcaagaagctgaagaaaggtgacttcgttgatgagtcctctgctaccaccgagagcgacctggaggagactttcaagaaactggttggcgacctgaacaaatctccagaagaaatcttcgacgctctgaagaaccagactgtggatctggtactgactgcgcatccgacccaaagcgtacgtcgcagcctgctgcagaaacacggtcgtatccgtgactgcctggctcagctgtacgcgaaagacatcactccagatgacaaacaagaactggatgaagccctgcaacgtgaaatccaagcagcatttcgcaccgacgagatcaaacgtactccaccgactccacaggacgaaatgcgtgcaggtatgtcttacttccacgagactatctggaaaggtgtgccgaagttcctgcgtcgtgttgataccgctctgaagaacatcggtatcgaagaacgcgtgccgtacaacgctccgctgattcagtttagctcctggatgggtggtgatcgtgatggcaatccacgcgtaactccagaggtgactcgtgacgtgtgtctgctggctcgtatgatggctgcaaccatgtacttcaaccagattgaggacctgatgttcgagatgtccatgtggcgttgcaacgacgagctgcgtgcacgtgcagatgaagtacacgctaactctcgtaaagatgctgctaaacactacatcgagttctggaaatccatcccgaccaccgagccgtaccgtgttatcctgggtgacgttcgtgacaaactgtatcatacccgtgaacgtgcacaccaactgctgtctaacggtcacagcgatgtaccagttgaagcgaccttcatcaacctggagcaattcctggaaccgctggaactgtgctaccgttccctgtgttcttgtggtgatcgtccgatcgcagacggttccctgctggacttcctgcgtcaagtgtccaccttcggtctgagcctggttcgcctggacatccgtcaggagagcgaccgtcacactgacgtgctggacgccatcactacccacctggacatcggctcctatcgtgaatggtctgaagaacgtcgccaggagtggctgctgtctgaactgtctggtaaacgtccgctgttcggctccgacctgccgaagaccgaggaaatcgctgatgtgctggataccttccacgtgatcgcagaactgccagcagacagctttggtgcatacatcatcagcatggctactgctccatccgatgttctggctgttgaactgctgcaacgtgaatgccgtgtgaagcaaccgctgcgtgttgtaccactgtttgagaaactggcagacctggaggctgctccagctgccgttgctcgtctgttcagcgttgattggtacaagaatcgtatcaatggtaaacaggaggttatgatcggctattccgacagcggtaaagacgctggtcgtctgtctgctgcttggcagctgtacaaagcgcaggaagaactggttaaggtggcgaaagaatacggtgttaagctgaccatgtttcatggtcgtggtggtactgtaggtcgtggtggtggtccaacccacctggcgatcctgtctcaaccaccggataccatcaacggctctctgcgtgttaccgttcagggtgaagttatcgaacagagcttcggtgaggagcacctgtgcttccgtaccctgcaacgcttcaccgcagcaactctggaacacggtatgcgtccaccaatctctccgaaaccggaatggcgtgcactgctggatgagatggctgtggttgctaccgaagaataccgtagcgtagtgttccaggaaccacgtttcgttgaatactttcgcctggccactccagagctggaatacggtcgtatgaacattggctctcgtccaagcaaacgcaaaccgtccggtggcatcgaatccctgcgtgcaatcccgtggatcttcgcgtggactcagactcgcttccacctgccagtgtggctgggtttcggttccgctattcgtcacgtgatcgagaaagacgttcgcaacctgcacatgctgcaagacatgtaccaacactggccattcttccgtgttactatcgacctgatcgaaatggtgttcgctaaaggtgatccaggtattgcagcgctgtacgataagctgctggtgtctgaagagctgtggccattcggtgagaaactgcgtgctaacttcgaagagactaagaagctgatcctgcaaaccgctggccacaaagacctgctggaaggtgatccatatctgaaacaacgtctgcgtctgcgtgactcttacatcaccaccctgaatgtatgtcaggcgtacaccctgaaacgtatccgtgacccgtcctaccacgttaccctgcgtccgcatatctccaaagaaatcgctgagagcagcaaaccagctaaggagctgatcgaactgaacccgacctctgagtacgcaccaggcctggaggacactctgattctgaccatgaaaggcattgcagcaggtctgcagaacactggttaagcggccgccatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccattttgtttaactttaagaaggagatggtaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttatgatttgtttaactttaagaaggagatctcgagatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataatttgtttaactttaagaaggagatactagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttaaacaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 10 - DNA, 23788 bp, synthetic construct
  source 1..23788: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggccaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttataattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataataacttaagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagtttaaaccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaacccgggccaccatggcaaaccgtaagctggagaagatggcatctatcgacgttcacctgcgtcagctggttccaggtaaggtaagcgaggacgacaaactggttgaatacgacgctctgctgctggatcgtttcctggacattctgcaagacctgcacggtgaagacctgcgtgagactgtacaagagctgtacgaacattctgccgaatacgagggtaagcacgaaccgaagaaactggaagagctgggtagcgtgctgacctctctggacccaggtgattccattgttatcgctaaagcattcagccacatgctgaacctggctaacctggctgaagaagtgcagatcgcctaccgtcgtcgtatcaagaagctgaagaaaggtgacttcgttgatgagtcctctgctaccaccgagagcgacctggaggagactttcaagaaactggttggcgacctgaacaaatctccagaagaaatcttcgacgctctgaagaaccagactgtggatctggtactgactgcgcatccgacccaaagcgtacgtcgcagcctgctgcagaaacacggtcgtatccgtgactgcctggctcagctgtacgcgaaagacatcactccagatgacaaacaagaactggatgaagccctgcaacgtgaaatccaagcagcatttcgcaccgacgagatcaaacgtactccaccgactccacaggacgaaatgcgtgcaggtatgtcttacttccacgagactatctggaaaggtgtgccgaagttcctgcgtcgtgttgataccgctctgaagaacatcggtatcgaagaacgcgtgccgtacaacgctccgctgattcagtttagctcctggatgggtggtgatcgtgatggcaatccacgcgtaactccagaggtgactcgtgacgtgtgtctgctggctcgtatgatggctgcaaccatgtacttcaaccagattgaggacctgatgttcgagatgtccatgtggcgttgcaacgacgagctgcgtgcacgtgcagatgaagtacacgctaactctcgtaaagatgctgctaaacactacatcgagttctggaaatccatcccgaccaccgagccgtaccgtgttatcctgggtgacgttcgtgacaaactgtatcatacccgtgaacgtgcacaccaactgctgtctaacggtcacagcgatgtaccagttgaagcgaccttcatcaacctggagcaattcctggaaccgctggaactgtgctaccgttccctgtgttcttgtggtgatcgtccgatcgcagacggttccctgctggacttcctgcgtcaagtgtccaccttcggtctgagcctggttcgcctggacatccgtcaggagagcgaccgtcacactgacgtgctggacgccatcactacccacctggacatcggctcctatcgtgaatggtctgaagaacgtcgccaggagtggctgctgtctgaactgtctggtaaacgtccgctgttcggctccgacctgccgaagaccgaggaaatcgctgatgtgctggataccttccacgtgatcgcagaactgccagcagacagctttggtgcatacatcatcagcatggctactgctccatccgatgttctggctgttgaactgctgcaacgtgaatgccgtgtgaagcaaccgctgcgtgttgtaccactgtttgagaaactggcagacctggaggctgctccagctgccgttgctcgtctgttcagcgttgattggtacaagaatcgtatcaatggtaaacaggaggttatgatcggctattccgacagcggtaaagacgctggtcgtctgtctgctgcttggcagctgtacaaagcgcaggaagaactggttaaggtggcgaaagaatacggtgttaagctgaccatgtttcatggtcgtggtggtactgtaggtcgtggtggtggtccaacccacctggcgatcctgtctcaaccaccggataccatcaacggctctctgcgtgttaccgttcagggtgaagttatcgaacagagcttcggtgaggagcacctgtgcttccgtaccctgcaacgcttcaccgcagcaactctggaacacggtatgcgtccaccaatctctccgaaaccggaatggcgtgcactgctggatgagatggctgtggttgctaccgaagaataccgtagcgtagtgttccaggaaccacgtttcgttgaatactttcgcctggccactccagagctggaatacggtcgtatgaacattggctctcgtccaagcaaacgcaaaccgtccggtggcatcgaatccctgcgtgcaatcccgtggatcttcgcgtggactcagactcgcttccacctgccagtgtggctgggtttcggttccgctattcgtcacgtgatcgagaaagacgttcgcaacctgcacatgctgcaagacatgtaccaacactggccattcttccgtgttactatcgacctgatcgaaatggtgttcgctaaaggtgatccaggtattgcagcgctgtacgataagctgctggtgtctgaagagctgtggccattcggtgagaaactgcgtgctaacttcgaagagactaagaagctgatcctgcaaaccgctggccacaaagacctgctggaaggtgatccatatctgaaacaacgtctgcgtctgcgtgactcttacatcaccaccctgaatgtatgtcaggcgtacaccctgaaacgtatccgtgacccgtcctaccacgttaccctgcgtccgcatatctccaaagaaatcgctgagagcagcaaaccagctaaggagctgatcgaactgaacccgacctctgagtacgcaccaggcctggaggacactctgattctgaccatgaaaggcattgcagcaggtctgcagaacactggttaagcatgctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagccgcggccaccatgccaccacagctgcaaaacggtctgaacctgagcgctaaagtggttcagggcagcctggattccctgccacaggctgttcgtgagttcctggagaacaacgcagagctgtgccaaccggatcacattcacatctgcgatggttccgaagaggagaacggtcgtctgctgggtcagatggaggaagaaggcatcctgcgtcgcctgaagaagtacgataactgttggctggctctgaccgatccacgtgatgtggctcgcatcgagtccaagactgttatcgttacccaagagcaacgcgatactgtgccgatcccgaagactggcctgagccagctgggtcgctggatgtctgaagaggactttgagaaagcgttcaacgctcgctttccaggttgcatgaaaggtcgtaccatgtacgtgatcccgttcagcatgggtccactgggttctccactgtctaagatcggcattgagctgactgactctccatacgttgttgcttctatgcgtatcatgacccgtatgggcactccagttctggaagcggttggtgatggcgagtttgtgaaatgtctgcactccgtgggctgtccactgccactgcagaaaccactggttaacaactggccatgcaacccagagctgaccctgatcgctcacctgccagatcgtcgtgaaatcatctcctttggttctggttatggtggtaactctctgctgggcaagaaatgcttcgctctgcgtatggcgagccgtctggcaaaggaagagggctggctggctgaacacatgctgattctgggcattaccaatccggagggtgagaagaagtacctggcagctgccttcccgtccgcttgtggtaagaccaatctggccatgatgaacccgtccctgccaggctggaaggtagagtgcgttggcgacgacattgcctggatgaagttcgatgcgcaaggccatctgcgtgccatcaatccggagaacggtttctttggtgttgctccaggtacttccgtaaagactaacccgaatgccatcaagaccatccagaagaacaccatcttcaccaacgtagcggaaacttccgacggtggcgtttactgggagggcattgatgaaccactggccagcggtgttactatcacctcctggaagaacaaagagtggagcagcgaagatggcgagccatgtgcccacccgaactctcgcttctgcactccagcctctcagtgtccaatcattgacgctgcgtgggagtctccagaaggcgttccgattgagggtatcatcttcggtggtcgtcgtccagcaggtgtgccactggtttacgaagctctgtcttggcagcacggtgtattcgttggtgctgcaatgcgctctgaggccaccgctgctgctgaacacaaaggcaagatcatcatgcacgatccgtttgcgatgcgtccgttcttcggttacaacttcggcaagtacctggctcactggctgagcatggctcagcatccagctgcgaaactgccgaagatttttcacgtaaactggtttcgtaaagacaaagaaggcaagttcctgtggccaggtttcggtgagaacagccgtgttctggagtggatgttcaatcgtatcgacggtaaagcatccaccaaactgactccgatcggctacatcccgaaggaggacgcactgaacctgaaaggtctgggtcatatcaacatgatggagctgttcagcatcagcaaagagttctgggagaaagaggttgaggacatcgagaagtacctggaggaccaggtgaacgcagacctgccgtgcgaaatcgaacgtgagatcctggcactgaaacagcgtatctctcaaatgtaactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 11 - DNA, 16377 bp, synthetic construct
  source 1..16377: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggtttaattaagtggcgcgccatggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgcgtctggccgttggtgccctgctggtgtgcgcggtgctgggcctgtgcctggccgtcccagataaaaccgttcgctggtgcgcagtcagtgagcatgaagcgacgaagtgccaatctttccgcgatcacatgaaaagcgtaattccgagcgatggtccgagtgtagcttgtgttaagaaagcaagctatctggactgtatccgcgcaattgcagcgaacgaagctgatgcagttaccctggacgcaggtctggtttacgacgcgtacctggctcctaacaatctgaaaccggttgtagcggagttctatggtagtaaagaagacccgcaaactttctattatgcagtggccgtggtaaagaaggactctggttttcagatgaaccagctgcgtgggaagaaaagttgtcatacgggcctggggcgttctgcgggttggaacattccaattgggctgctgtattgcgatctgccggaaccacgcaagccgctggagaaagctgtagcgaacttcttcagtggttcttgtgcgccttgcgccgatggtactgattttccgcagctgtgtcagctgtgtccgggctgcggttgttctaccctgaatcagtactttggctatagtggggcgtttaaatgcctgaaagatggggcgggtgacgtggcgttcgtcaaacattctacgattttcgaaaacctggcgaacaaagcagatcgtgaccaatatgaactgctgtgtctggacaacactcgcaagccagtcgatgaatataaagattgtcatctggcacaagtgcctagtcatactgtggtcgcgcgtagcattggtggtaaggaggacctgatttgggaactgctgaaccaagctcaggagcatttcggcaaagataaaagcaaagaatttcagctgttttctagcccgcacggcaaagacctgctgtttaaagacagcgcccacggctttctgaaagtgcctccacgcatggatgccaaaatgtatctgggttatgaatatgttacggcaattcgcaatctgcgtgaaggcacgtgcccggaagctccgactgacgagtgcaaaccagtaaagtggtgtgccctgtctcatcatgagcgcctgaaatgtgatgaatggagtgtgaactctgttggcaaaattgagtgcgttagtgctgaaaccaccgaggactgtatcgcaaagatcatgaacggcgaagcagatgctatgtctctggatggcggttttgtgtatatcgcaggtaaatgcggcctggtcccagttctggctgaaaattataacaaaagtgataactgtgaggatactccaggggcgggctattttgcggtcgctgtcgtcaagaaatctgcgagcgatctgacatgggataacctgaaagggaagaaatcttgccataccgcggttggccgcaccgctgggtggaacatcccgatgggcctgctgtataacaaaatcaatcattgccgttttgacgagttcttcagtgaggggtgtgcgcctggtagtaagaaagatagcagcctgtgcaaactgtgcatgggcagcggcctgaatctgtgcgaacctaacaataaagagggttactacggctacaccggcgcgtttcgctgcctggttgagaaaggtgatgttgcgtttgtaaagcaccaaacagtaccgcagaatacgggtgggaagaatccggacccgtgggccaagaatctgaatgaaaaggattacgaactgctgtgcctggatgggacccgcaagccggttgaagaatacgcgaattgtcacctggcccgcgccccgaatcacgccgtggtgacgcgcaaagataaagaggcctgcgtccacaaaatcctgcgtcagcagcagcacctgttcggcagcaatgtgacagattgtagcggtaatttctgtctgttccgtagcgaaaccaaggacctgctgttccgtgacgacaccgtgtgtctggccaaactgcacgaccgtaatacctacgagaaatacctgggcgaggagtacgtgaaagccgtgggcaatctgcgtaagtgtagcacaagcagcctgctggaagcctgcacatttcgtcgtccgtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 12 - DNA, 16292 bp, synthetic construct
  source 1..16292: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatggaggtcgaaagagtccaagacatttcatcttcttctctactaacagaagcaatcccgttggagttcatcagatcagagaaagaacaaccagcgatcacaacattccgaggtccaacgccggcgattcccgtcgtcgatctaagcgatcccgacgaagaaagcgtgaggcgcgcggtggtgaaagcgagtgaagaatgggggctattccaagtggttaaccacgggattccgacggagctgatacgacgtttacaagacgtcggaagaaaattcttcgagcttccttcgtcggagaaagaatccgtcgctaaaccggaagattcgaaagacattgaaggatacggaacaaagcttcagaaagatccagaaggtaaaaaagcttgggtcgatcatctcttccatcgaatctggccaccgtcatgcgtcaattacagattctggcctaagaatccacctgaatacagggaggtgaatgaagagtatgcagtgcatgtgaagaagctatcggagacgttattagggattctctcggatggattagggttaaagcgtgatgcgttgaaagaaggtctcggcggagagatggcggagtatatgatgaagattaactattatccgccgtgtcctcggccggatttagctttaggtgtaccggctcatacagatctcagtggaatcactcttcttgttcctaacgaagttcctggacttcaagttttcaaagatgatcactggttcgatgcagagtatattccctccgccgtcattgttcacatcggcgatcagattctgaggttgagtaatgggaggtataaaaatgtgttgcataggacgacggtggataaagagaagacgaggatgtcgtggccggttttcttggagcctccccgtgaaaagattgttggacctttaccggaactaaccggagatgataatcctccaaagtttaaaccgtttgctttcaaggattacagttaccgcaagctcaataaacttcctctggattgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccatggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgtcaaaggaacagaacggtacagagaagccacagaatttgagtagaagggacgtcttgaagggtattgcaatcacagcaggtgttgttgctgctggcgctgtagtgggagttaaccctattggtgctgctcatgctgctggtaagtgcccaggtatcactcctaaggcttcattacaataccaaccacaccctaaaggtaaagagcaatgctctgcctgtgcaaacttcatcgcaccacattgttgtaaagtggttgctggttctgttgttccagaaggatattgtatggccttcatcttgaagcctgcataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 13 - DNA, 16760 bp, synthetic construct
  source 1..16760: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccatggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgtcaaaggaacagaacggtacagagaagccacagaatttgagtagaagggacgtcttgaagggtattgcaatcacagcaggtgttgttgctgctggcgctgtagtgggagttaaccctattggtgctgctcatgctgctggtaagtgcccaggtatcactcctaaggcttcattacaataccaaccacaccctaaaggtaaagagcaatgctctgcctgtgcaaacttcatcgcaccacattgttgtaaagtggttgctggttctgttgttccagaaggatattgtatggccttcatcttgaagcctgcataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 14 - DNA, 17194 bp, synthetic construct
  source 1..17194: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggccaccatgtctaagaaacatcctgtgattgctgtgactggtagttctggtgccggaacaacaaccgttaaacatgcattccatgatatcttcagaagattgaagattgacccagttgtcattgaaggagattcatttcacaggtacaacaggaacgaaatgagggaagcaattgccaaagctgccgctgatggaaagactatttctcatttcggtccagaaggaaacgactttgaagcattagaacgtttgttccgtgaatatggtgaatctggtcatggacaaatgcgttactacgttcacgacgaattggaggctgagttacgtggttctgcacccggaaccttcactccttggcaggacattccattgggtactgacttgttattctacgaaggtttacatggtggtgtgaagacaggtgccgtggacgttactaactacgtagatttgttagttggtgtagttcccgtggtcaatttggaatggatacagaagatccatcgtgataacgctcaaagaggctactcagccgaagccattgtagataccatcttgagaaggatgcctgactacatccactacattacacctcagttcagtagggctcacataaacttccaacgtgtacctttagtcgatacttctaaccctttcatagctagggatataccaactcctgatgagagtatggttgtgataagattcagagatccaaaggaagagaactttcccactttgttacaaatgttacccggttcctttatgtcaaggtcaaatactttagtgattccaggcacaaagatgggttatgctatggaattgatcttgggaccacgtattgaaagaatgttggaagatatgcatttgaccatttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgtgtgatgaggacgaaactacagctttagtgtgcgataatggatcaggtttagtcaaagctggctttgctggtgatgatgcaccacgtgctgtcttcccttcaattgtaggtagacccagacatcagggagttatggttggtatgggtcagaaagatagttatgttggagacgaggcacaatccaagcgtggaatcttaactttgaagtaccctattgagcacggtatcataacaaattgggatgacatggagaagatatggcatcataccttctataacgaattgagagtggctcctgaagagcatcccactttgttgacagaagctccattgaatcccaaagccaatagagagaagatgacacagatcatgttcgaaacattcaacgtccctgctatgtatgttgccattcaggcagtgttatctttgtacgcctcaggtagaactactggcatagtattggatagtggtgatggcgtgacacataacgttccaatctacgaaggttatgcattaccacacgctatcatgagattggatttggctggtagagacttaactgattacttaatgaagatattgacagaacgtggttactcctttgtcacaacagccgaacgtgaaattgttagagacattaaggagaaattgtgctacgtcgccttggatttcgagaacgaaatggctactgccgcttcttcctcctcattggagaaatcttatgaattacctgacggacaagtgattactattggtaatgaaagattccgttgtccagagacattgtttcaaccatcctttatcggcatggagagtgcaggcatccatgaaacaacttacaattccattatgaaatgtgacatagacattagaaaggacttatacgctaacaacgttatgtctggcggtactactatgtatcctggtattgcagaccgtatgcagaaggaaatcacagcattggcaccttctactatgaagatcaagataatcgcaccacctgaaaggaaatactctgtttggattggtggctcaatcttggcctctttatcaacctttcaacaaatgtggataactaagcaagaatacgacgaagctggtccctctattgtccataggaaatgcttctaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgtcaaaggaacagaacggtacagagaagccacagaatttgagtagaagggacgtcttgaagggtattgcaatcacagcaggtgttgttgctgctggcgctgtagtgggagttaaccctattggtgctgctcatgctgctggtaagtgcccaggtatcactcctaaggcttcattacaataccaaccacaccctaaaggtaaagagcaatgctctgcctgtgcaaacttcatcgcaccacattgttgtaaagtggttgctggttctgttgttccagaaggatattgtatggccttcatcttgaagcctgcataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 15 - DNA, 18631 bp, synthetic construct
  source 1..18631: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggccaccatgtctaagaaacatcctgtgattgctgtgactggtagttctggtgccggaacaacaaccgttaaacatgcattccatgatatcttcagaagattgaagattgacccagttgtcattgaaggagattcatttcacaggtacaacaggaacgaaatgagggaagcaattgccaaagctgccgctgatggaaagactatttctcatttcggtccagaaggaaacgactttgaagcattagaacgtttgttccgtgaatatggtgaatctggtcatggacaaatgcgttactacgttcacgacgaattggaggctgagttacgtggttctgcacccggaaccttcactccttggcaggacattccattgggtactgacttgttattctacgaaggtttacatggtggtgtgaagacaggtgccgtggacgttactaactacgtagatttgttagttggtgtagttcccgtggtcaatttggaatggatacagaagatccatcgtgataacgctcaaagaggctactcagccgaagccattgtagataccatcttgagaaggatgcctgactacatccactacattacacctcagttcagtagggctcacataaacttccaacgtgtacctttagtcgatacttctaaccctttcatagctagggatataccaactcctgatgagagtatggttgtgataagattcagagatccaaaggaagagaactttcccactttgttacaaatgttacccggttcctttatgtcaaggtcaaatactttagtgattccaggcacaaagatgggttatgctatggaattgatcttgggaccacgtattgaaagaatgttggaagatatgcatttgaccatttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgtgtgatgaggacgaaactacagctttagtgtgcgataatggatcaggtttagtcaaagctggctttgctggtgatgatgcaccacgtgctgtcttcccttcaattgtaggtagacccagacatcagggagttatggttggtatgggtcagaaagatagttatgttggagacgaggcacaatccaagcgtggaatcttaactttgaagtaccctattgagcacggtatcataacaaattgggatgacatggagaagatatggcatcataccttctataacgaattgagagtggctcctgaagagcatcccactttgttgacagaagctccattgaatcccaaagccaatagagagaagatgacacagatcatgttcgaaacattcaacgtccctgctatgtatgttgccattcaggcagtgttatctttgtacgcctcaggtagaactactggcatagtattggatagtggtgatggcgtgacacataacgttccaatctacgaaggttatgcattaccacacgctatcatgagattggatttggctggtagagacttaactgattacttaatgaagatattgacagaacgtggttactcctttgtcacaacagccgaacgtgaaattgttagagacattaaggagaaattgtgctacgtcgccttggatttcgagaacgaaatggctactgccgcttcttcctcctcattggagaaatcttatgaattacctgacggacaagtgattactattggtaatgaaagattccgttgtccagagacattgtttcaaccatcctttatcggcatggagagtgcaggcatccatgaaacaacttacaattccattatgaaatgtgacatagacattagaaaggacttatacgctaacaacgttatgtctggcggtactactatgtatcctggtattgcagaccgtatgcagaaggaaatcacagcattggcaccttctactatgaagatcaagataatcgcaccacctgaaaggaaatactctgtttggattggtggctcaatcttggcctctttatcaacctttcaacaaatgtggataactaagcaagaatacgacgaagctggtccctctattgtccataggaaatgcttctaacttaagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgtcaaaggaacagaacggtacagagaagccacagaatttgagtagaagggacgtcttgaagggtattgcaatcacagcaggtgttgttgctgctggcgctgtagtgggagttaaccctattggtgctgctcatgctgctggtaagtgcccaggtatcactcctaaggcttcattacaataccaaccacaccctaaaggtaaagagcaatgctctgcctgtgcaaacttcatcgcaccacattgttgtaaagtggttgctggttctgttgttccagaaggatattgtatggccttcatcttgaagcctgcataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 16 - DNA, 13637 bp, synthetic construct
  source 1..13637: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgaggccaccatgaagctgaccgccatctttccgcttcttttcactgctgtcgggtactgtgctgcccaaagcattgctgacttggcagcagccaatctcagcactgaggattccaagtcagctcagctcatctcagcagactcctcggatgacgcatctgattcctctgtcgagtctgtcgacgccgcctcctctgacgtctctggttcctctgtcgaatctgtcgacgtctctggttcctctctggaatccgttgacgtctctggttcctctctggagtccgtcgacgactccagtgaggactccgaagaggaagaacttcgtatcttgtcttccaagaaatccggatcctactactcctacggcaccaagaagtcgggatcctactcgggatactccacaaagaaatccgcatcgcgtcgcatcttgtcctccaagaaatcgggatcctactcgggatattccacaaagaaatccggatcgcgtcgcatcttgtcctccaagaaatcgggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtccgggggtctcagaggctccatgtaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 17 - DNA, 15564 bp, synthetic construct
  source 1..15564: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggccaccatggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaacccgggtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagccgcggccaccatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgaggccaccatgaagctgaccgccatctttccgcttcttttcactgctgtcgggtactgtgctgcccaaagcattgctgacttggcagcagccaatctcagcactgaggattccaagtcagctcagctcatctcagcagactcctcggatgacgcatctgattcctctgtcgagtctgtcgacgccgcctcctctgacgtctctggttcctctgtcgaatctgtcgacgtctctggttcctctctggaatccgttgacgtctctggttcctctctggagtccgtcgacgactccagtgaggactccgaagaggaagaacttcgtatcttgtcttccaagaaatccggatcctactactcctacggcaccaagaagtcgggatcctactcgggatactccacaaagaaatccgcatcgcgtcgcatcttgtcctccaagaaatcgggatcctactcgggatattccacaaagaaatccggatcgcgtcgcatcttgtcctccaagaaatcgggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtccgggggtctcagaggctccatgtaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 18 - DNA, 14634 bp, synthetic construct
  source 1..14634: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtactgttccaatacaacttttgttttgtttgaaagagcaaaatcagaaaaaaattaactcacatagatgggcatttgaaggctttgcagaattactgcgtcccaatatcgttacattgttagatgctggtactatgccaggtaaagattctatttaccagttatggagagagttcaggaatccaaatgttggtggcgcatgtggtgaaataagaactgatttgggtaagagatttgtaaagttgttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctagtctttaggattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaggctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttagctaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgagatgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 19 - DNA, 16558 bp, synthetic construct
  source 1..16558: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaacccgggtaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcgcgccaccatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtactgttccaatacaacttttgttttgtttgaaagagcaaaatcagaaaaaaattaactcacatagatgggcatttgaaggctttgcagaattactgcgtcccaatatcgttacattgttagatgctggtactatgccaggtaaagattctatttaccagttatggagagagttcaggaatccaaatgttggtggcgcatgtggtgaaataagaactgatttgggtaagagatttgtaaagttgttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctagtctttaggattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaggctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttagctaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgagatgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 20 - DNA, 12544 bp, synthetic construct
  source 1..12544: mol_type = other DNA
ggggaattgtgagcggataacaattcccctctagaaataattttgtttaactttaagaaggagatataccatggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaaggatccagaaataattttgtttaactttaagaaggagagaattcatgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttagctaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtactgttccaatacaacttttgttttgtttgaaagagcaaaatcagaaaaaaattaactcacatagatgggcatttgaaggctttgcagaattactgcgtcccaatatcgttacattgttagatgctggtactatgccaggtaaagattctatttaccagttatggagagagttcaggaatccaaatgttggtggcgcatgtggtgaaataagaactgatttgggtaagagatttgtaaagttgttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctagtctttaggattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaggctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaaggtaccagaaataattttgtttaactttaagaaggagactcgagatgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaattaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 21 - DNA, 14634 bp, synthetic construct; identical to SEQ ID NO: 18
  source 1..14634: mol_type = other DNA

SEQ ID NO: 22 - DNA, 16558 bp, synthetic construct; identical to SEQ ID NO: 19
  source 1..16558: mol_type = other DNA

SEQ ID NO: 23 - DNA, 12544 bp, synthetic construct; identical to SEQ ID NO: 20
  source 1..12544: mol_type = other DNA

SEQ ID NO: 24 - DNA, 20014 bp, synthetic construct
  source 1..20014: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtactgttccaatacaacttttgttttgtttgaaagagcaaaatcagaaaaaaattaactcacatagatgggcatttgaaggctttgcagaattactgcgtcccaatatcgttacattgttagatgctggtactatgccaggtaaagattctatttaccagttatggagagagttcaggaatccaaatgttggtggcgcatgtggtgaaataagaactgatttgggtaagagatttgtaaagttgttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctagtctttaggattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaggctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttagctaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgagatgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggcgaagtttggtagccgtaataagtctccgaagtggatctctaacggctgctgcttcctgctgggtgcctttactgctctgctgctgctgtggggtctgtgctcctttattatcccgattccgaacaccgatccgaagctgaactctgtagcgacctctctgcgttctctgaacttcccgaagaacccagcagctaccctgccaccgaacctgcagcatgatccaccggatactactttctacgacgatccggaaacctcttatactatggataaaccgatgaagaactgggacgagaaacgtaaggaatggctgctgcatcacccgtccttcggcgcagctgctcgtgacaagatcctgctggtaaccggttcccagccgaaacgctgtcacaacccgattggtgatcacctgctgctgcgtttctttaagaacaaagtggattactgccgtctgcataattacgatatcatctataacaacgcactgctgcacccgaaaatgaactcctattgggctaagtacccagtgatccgtgctgcaatgatggctcatccggaagttgaatgggtgtggtgggttgactctgacgctgtattcactgacatggagtttaaactgccactgaaacgctataagaaccacaatctggtggtgcatggttgggaaggtctggtacgtctgaaccactcctggactggtctgaacgcaggtgtattcctgatccgtaattgccaatggagcctggagttcatggatgtatgggttagcatgggtccacaaactccagaatacgagaaatggggtgagcgtctgcgcgaaaccttcaaagataaagttctgccagactctgatgaccagactgcactggcatacctgatcgcaaccgataacaaagacacctggcgtgagaaaatcttcctggaaagcgagtactacttcgaaggctattggctggaaatcgtaaagacttacgagaacatctccgaacgttacgatgaagttgaacgcaaagtagaaggtctgcgtcgtcgccacgctgagaaggttagcgagaaatacggtgcaatgcgtgaagaatacctgaaagataacaaacgtcgtccgttcatcacccatttcactggttgccagccgtgcaacggtcaccataacccagcatacaatgcgaacgactgttggaatggtatggaacgtgcactgaactttgcagataaccagatcctgcgtacctacggttaccatcgtcagaatctgctggacaagtccgtttctccactgccgttcggttatccagcagcttaacccgggtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagcgtacgccaccatggcttcgccggcaccggcgggcgatgccgtgtatgcggccaatggcggcctcactgacccgcttctagtgagcgcgaacggccatggtgccgccgcgaggaaggctggccacggcgccaggggcaggtactgggtggcctccgacaaggccgagaggcgcgctgccaaggagtccggcggtgaggacggccgcgcgctgctgttccggaagtataaggtcaaaggcgccctcctgcacccctacaggttgctgatcatcatccgattagtcgctgtcctcgccttcttcgcgtggcgcatcaggcacaacaaatccgacatcatgtggttctggacaatgtccatcgtcggcgacgtctggttcggcttctcgtggctcctcaaccagctcccaaagttcaaccctgtgaagaccatcccagatctcgccgccctccagcggcactttggctaccctgacggcggcgcctctaggctccccggtatcgatgttttcgtcaccactgccgaccccattgatgagcccatactctacaccatgaactgtgtcctctccatcctgtctgtcgattacccagtcgatcggctagcctgctatctctcagatgatagtggggcgctggttctctatgaggcactggccgaggtcggaaagtttgcgcctctatgggttccgttctgccgcaagtactccattgagccaagggcaccagaaagctattttgagcatgtggcaccgccgcaggctggaagggtgacgcaggagttcttgaatgattataggagggtgcagatggagtacgatgagttcaaggcgcgcttggacaacctccctgatgccatacgcaagagatccgatgtttacaatagcgtgagagatgcaggaggagctcaaaaggcaacttggatggcaaatgggacgcagtggcccggcacatggattgatccagcagagaaccataggaaaggacaccatgctccaattgccaaggttgtgctgaaccatccaagccgtggacaacaccctattactgaaagcaatcccagcattgccaccactgatgagcgcctcccaatgcttgtttacgtctctcgtgagaagaacccaggttatgaccacaacaagaaggcaggtgccctgaatgcacagctgcgggcctctgctctcctctctaatgcccaactcatcatcaacttcgactgcgaccactacatcaacaactctcaagccctaagctcggctgtgtgcttcatgctggatcaacgggacggtgataacaccgcatttgttcagttcccacagcgcttcgacaatgttgaccccacggaccgctatggtaaccacaatcgtgtcttctttgatgggaccatgcttgcccttaatggtttgcaagggccctcatacctcggtactggttgcatgttccgtcgcttagcactctatggtattgacccaccccactgtagagcagaaaacatcacggccgaagctagcaggtttggcaactccacaatcttcctagattcagtgtcaaaagccctgaaaaatgacaggacaatcacaccgccaccaattgatgacacattccttgctgagctagagagggttgtgacatgttcttatgacaaaggtactgactggggcaagggtgtagggtacatctatgacatagccactgaagatatagtaacaggattccgcatccacgggcaggggtggcgctctatgtattgcacaatggagcacgatgcgttctgtggcgttgcaccaatcaacctaactgaacgcctccatcaaattgtgcgctggtctggtgggtctttagagatgttcttctcccacaacaatccattcatcggtggtcgccggattcaaccccttcagcgtgtctcctacctcaacatgacagtctacccagtcacatcagtcttcatcctaatctatgctctaagcccggtgatgtggcttatccccgatgaagtatacatccagagaccattcactaggtatgtcgtgtaccttcttgtaatcatcgtgatgattcacatgattggctggcttgagataaagtgggcgggggtcacatggctggactattggcgcaatgagcagttctttatgatcggctcaacaagtgcatatccaatggcagtgctgcacatggcagtgaacctcctcacaaagaagggcatacacttcagggtcacttccaagcaaacagccgcggacgacaatgacaagttcgccgacctttatgattttcgatgggtgccaatgctcatcccaacaatggcagttctgatctgcaatgttggtgccattggtgtagccctgggcaaaactgtggtatacattggaacatggacagcggcgaagaagatgcatgcagcactgggtctgctgttcaacatatggatcatgtttctcctctacccatttgcactagcgatcatggggcggtgggcgaagaggccaatcatccttgtagttctgctgccggttgtctttgcacttgtcgcgctgttgtatgtaggtatccatatcctacttgctggtctgattccattctaggtagtatagaaaacattgtaatttcttgtgtagttaccaaggaaccaattatgttttttttagcaaaaaaaaaaaaaaaaaaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 25 - DNA, 20014 bp, synthetic construct; identical to SEQ ID NO: 24
  source 1..20014: mol_type = other DNA

SEQ ID NO: 26 - DNA, 13165 bp, synthetic construct
  source 1..13165: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggcgcgcctttgtttaactttaagaaggagagctcaatatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaatttgtttaactttaagaaggagatgctagcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagcggccgccatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccattttgtttaactttaagaaggagatggtaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttatgatttgtttaactttaagaaggagatctcgagatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataatttgtttaactttaagaaggagatactagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttaaacaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 27 - DNA, 17840 bp, synthetic construct
  source 1..17840: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggccaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttataattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataataacttaagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 28 - DNA, 19736 bp, synthetic construct
  source 1..19736: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggccaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttataattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataataacttaagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagcccggggccaccatgcgtttccgtctgaacatcgtactggctaccatgatcttctcttctctggctgtttctaccgctatggcaggcgtgcctgtgcagtggtccgttgacgtgcacgcaccgatctacaatcctcctcgcgttgcggacggtgtagcatacttcgatactgcccagtccaaaggtccaaacgtgttctctgcgaagaacggcaagatcctgtggcgtttcaccacgggtggcaccatcctgatgccactgaccgtaggccacggtcaggtgtgggtagcgtctgacgtgggctctacccattacctgcgtgcgatcgaagctaagactggtaaactgatctgggactacacccgtcacgaaccaccggaatgcatgtgttcccacctgacccattacgaacatcacctgctgtttgcgcagaccgatggccactctctgtatgcgttctatcctgtcggcaacatcccgaaccgtcgcctgtgggctttcactggtgatggtgctaaactgactgcaccagttgtggtggatcacaccgtaatcttcggttctgctgatcacggtgtttacggtctgttcgacaagactggcaagatccgttggcagcagaagacgggttatggtttcatggcacagcctgcggtgtggaaacacgaagtgatcatcggcaaccgtggtggtactgttcacgcgtactctaccaccactggcacctctctgtggaacttcacgaccaatggtcctatcaacacgactgccctgatctggcatgattctgccttcatcgcttccggtgcaggtgatcgtggcgtttacgctctgtccgctaagactggcaaacaactgtggtacactcgcatggcagattacactgcgtatgctccggttatggcgaaacagattgtagtggttgcttctcaggacggtaacatgctgggtctgtctgcgaccactggcaaagttctgtggcgttccgaactgcacggtatccctaagtctcaacctgctctgtggaagggtgatgcggtcctgaaagttaacgatcacaagatcatggctttcaatgctcagtctggtggcctggtttggacttatcactccaagaacgttgttacctctccggtgccgaaaggtgatagcgtctatgttggcaccagcggtggcactctggtggcgatcggtcagtaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 29 - DNA, 14382 bp, synthetic construct
  source 1..14382: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggcgcgcctttgtttaactttaagaaggagatataccatgcgtttccgtctgaacatcgtactggctaccatgatcttctcttctctggctgtttctaccgctatggcaggcgtgcctgtgcagtggtccgttgacgtgcacgcaccgatctacaatcctcctcgcgttgcggacggtgtagcatacttcgatactgcccagtccaaaggtccaaacgtgttctctgcgaagaacggcaagatcctgtggcgtttcaccacgggtggcaccatcctgatgccactgaccgtaggccacggtcaggtgtgggtagcgtctgacgtgggctctacccattacctgcgtgcgatcgaagctaagactggtaaactgatctgggactacacccgtcacgaaccaccggaatgcatgtgttcccacctgacccattacgaacatcacctgctgtttgcgcagaccgatggccactctctgtatgcgttctatcctgtcggcaacatcccgaaccgtcgcctgtgggctttcactggtgatggtgctaaactgactgcaccagttgtggtggatcacaccgtaatcttcggttctgctgatcacggtgtttacggtctgttcgacaagactggcaagatccgttggcagcagaagacgggttatggtttcatggcacagcctgcggtgtggaaacacgaagtgatcatcggcaaccgtggtggtactgttcacgcgtactctaccaccactggcacctctctgtggaacttcacgaccaatggtcctatcaacacgactgccctgatctggcatgattctgccttcatcgcttccggtgcaggtgatcgtggcgtttacgctctgtccgctaagactggcaaacaactgtggtacactcgcatggcagattacactgcgtatgctccggttatggcgaaacagattgtagtggttgcttctcaggacggtaacatgctgggtctgtctgcgaccactggcaaagttctgtggcgttccgaactgcacggtatccctaagtctcaacctgctctgtggaagggtgatgcggtcctgaaagttaacgatcacaagatcatggctttcaatgctcagtctggtggcctggtttggacttatcactccaagaacgttgttacctctccggtgccgaaaggtgatagcgtctatgttggcaccagcggtggcactctggtggcgatcggtcagtaatttgtttaactttaagaaggagagctcaatatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaatttgtttaactttaagaaggagatgctagcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagcggccgccatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccattttgtttaactttaagaaggagatggtaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttatgatttgtttaactttaagaaggagatctcgagatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataatttgtttaactttaagaaggagatactagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttaaacaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 30 - DNA, 17840 bp, synthetic construct; identical to SEQ ID NO: 27
  source 1..17840: mol_type = other DNA